SEQUENCE LISTING (43 sequences)

SEQ ID NO: 1 - DNA, 1788 bp, Mus musculus
  CDS (223)...(1515): mIk-2
aattcgttctaccttctctgaaccccagtggtgtgtcaaggccggactgggagcttgggggaagaggaagaggaagaggaatctgcggctcatccagggatcagggtccttcccaagtggccactcagaggggactcagagcaagtctagatttgtgtggcagagagagacagctctcgtttggccttggggaggcacaagtctgttgataacctgaagacaatggatgtcgatgagggtcaagacatgtcccaagtttcaggaaaggagagccccccagtcagtgacactccagatgaaggggatgagcccatgcctgtccctgaggacctgtccactacctctggagcacagcagaactccaagagtgatcgaggcatgggtgaacggcctttccagtgcaaccagtctggggcctcctttacccagaaaggcaacctcctgcggcacatcaagctgcactcgggtgagaagcccttcaaatgccatctttgcaactatgcctgccgccggagggacgccctcaccggccacctgaggacgcactccgttggtaagcctcacaaatgtggatattgtggccggagctataaacagcgaagctctttagaggagcataaagagcgatgccacaactacttggaaagcatgggccttccgggcgtgtgcccagtcattaaggaagaaactaaccacaacgagatggcagaagacctgtgcaagataggagcagagaggtcccttgtcctggacaggctggcaagcaatgtcgccaaacgtaagagctctatgcctcagaaatttcttggagacaagtgcctgtcagacatgccctatgacagtgccaactatgagaaggaggatatgatgacatcccacgtgatggaccaggccatcaacaatgccatcaactacctgggggctgagtccctgcgcccattggtgcagacaccccccggtagctccgaggtggtgccagtcatcagctccatgtaccagctgcacaagcccccctcagatggccccccacggtccaaccattcagcacaggacgccgtggataacttgctgctgctgtccaaggccaagtctgtgtcatcggagcgagaggcctccccgagcaacagctgccaagactccacagatacagagagcaacgcggaggaacagcgcagcggccttatctacctaaccaaccacatcaacccgcatgcacgcaatgggctggctctcaaggaggagcagcgcgcctacgaggtgctgagggcggcctcagagaactcgcaggatgccttccgtgtggtcagcacgagtggcgagcagctgaaggtgtacaagtgcgaacactgccgcgtgctcttcctggatcacgtcatgtataccattcacatgggctgccatggctgccatggctttcgggatccctttgagtgtaacatgtgtggttatcacagccaggacaggtacgagttctcatcccatatcacgcggggggagcatcgttaccacctgagctaaacccagccaggccccactgaagcacaaagatagctggttatgcctccttcccggcagctggacccacagcggacaatgtgggagtggatttgcaggcagcatttgttcttttatgttggttgtttggcgtttcatttgcgttggaagataagtttttaatgttagtgacaggattgcattgcatcagcaacattcacaacatccatccttctagccagttttgttcactggtagctgaggtttcccggatatgtggcttcctaacactct

SEQ ID NO: 2 - DNA, 1386 bp, Homo sapiens
  CDS (1)...(1383): hIk-1
aatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccgttggtaaacctcacaaatgtggatattgtggccgaagctataaacagcgaacgtctttagaggaacataaagagcgctgccacaactacttggaaagcatgggccttccgggcacactgtacccagtcattaaagaagaaactaagcacagtgaaatggcagaagacctgtgcaagataggatcagagagatctctcgtgctggacagactagcaagtaatgtcgccaaacgtaagagctctatgcctcagaaatttcttggggacaagggcctgtccgacacgccctacgacagtgccacgtacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaggcgctcggagggcaccccgcgctccaaccactcggcccaggacagcgccgtggagtacctgctgctgctctccaaggccaagttggtgccctcggagcgcgaggcgtccccgagcaacagctgccaagactccacggacaccgagagcaacaacgaggagcagcgcagcggtcttatctacctgaccaaccacatcgcccgacgcgcgcaacgcgtgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagctaa

SEQ ID NO: 3 - DNA, 1296 bp, Mus musculus
  CDS (1)...(1296): mIk-3
atggatgtcgatgagggtcaagacatgtcccaagtttcaggaaaggagagccccccagtcagtgacactccagatgaaggggatgagcccatgcctgtccctgaggacctgtccactacctctggagcacagcagaactccaagagtgatcgaggcatggccagtaatgttaaagtagagactcagagtgatgaagagaatgggcgtgcctgtgaaatgaatggggaagaatgtgcagaggatttacgaatgcttgatgcctcgggagagaaaatgaatggctcccacagggaccaaggcagctcggctttgtcaggagttggaggcattcgacttcctaacggaaaactaaagtgtgatatctgtgggatcgtttgcatcgggcccaatgtgctcatggttcacaaaagaagtcatactggtgaacggcctttccagtgcaaccagtctggggcctcctttacccagaaaggcaacctcctgcggcacatcaagctgcactcgggtgagaagcccttcaaatgccatctttgcaactatgcctgccgccggagggacgccctcaccggccacctgaggacgcactccggagacaagtgcctgtcagacatgccctatgacagtgccaactatgagaaggaggatatgatgacatcccacgtgatggaccaggccatcaacaatgccatcaactacctgggggctgagtccctgcgcccattggtgcagacaccccccggtagctccgaggtggtgccagtcatcagctccatgtaccagctgcacaagcccccctcagatggccccccacggtccaaccattcagcacaggacgccgtggataacttgctgctgctgtccaaggccaagtctgtgtcatcggagcgagaggcctccccgagcaacagctgccaagactccacagatacagagagcaacgcggaggaacagcgcagcggccttatctacctaaccaaccacatcaacccgcatgcacgcaatgggctggctctcaaggaggagcagcgcgcctacgaggtgctgagggcggcctcagagaactcgcaggatgccttccgtgtggtcagcacgagtggcgagcagctgaaggtgtacaagtgcgaacactgccgcgtgctcttcctggatcacgtcatgtataccattcacatgggctgccatggctgccatggctttcgggatccctttgagtgtaacatgtgtggttatcacagccaggacaggtacgagttctcatcccatatcacgcggggggagcatcgttaccacctgagc

SEQ ID NO: 4 - DNA, 2049 bp, Mus musculus
  CDS (223)...(1776): mIk-1
aattcgttctaccttctctgaaccccagtggtgtgtcaaggccggactgggagcttgggggaagaggaagaggaagaggaatctgcggctcatccagggatcagggtccttcccaagtggccactcagaggggactcagagcaagtctagatttgtgtggcagagagagacagctctcgtttggccttggggaggcacaagtctgttgataacctgaagacaatggatgtcgatgagggtcaagacatgtcccaagtttcaggaaaggagagccccccagtcagtgacactccagatgaaggggatgagcccatgcctgtccctgaggacctgtccactacctctggagcacagcagaactccaagagtgatcgaggcatggccagtaatgttaaagtagagactcagagtgatgaagagaatgggcgtgcctgtgaaatgaatggggaagaatgtgcagaggatttacgaatgcttgatgcctcgggagagaaaatgaatggctcccacagggaccaaggcagctcggctttgtcaggagttggaggcattcgacttcctaacggaaaactaaagtgtgatatctgtgggatcgtttgcatcgggcccaatgtgctcatggttcacaaaagaagtcatactggtgaacggcctttccagtgcaaccagtctggggcctcctttacccagaaaggcaacctcctgcggcacatcaagctgcactcgggtgagaagcccttcaaatgccatctttgcaactatgcctgccgccggagggacgccctcaccggccacctgaggacgcactccgttggtaagcctcacaaatgtggatattgtggccggagctataaacagcgaagctctttagaggagcataaagagcgatgccacaactacttggaaagcatgggccttccgggcgtgtgcccagtcattaaggaagaaactaaccacaacgagatggcagaagacctgtgcaagataggagcagagaggtcccttgtcctggacaggctggcaagcaatgtcgccaaacgtaagagctctatgcctcagaaatttcttggagacaagtgcctgtcagacatgccctatgacagtgccaactatgagaaggaggatatgatgacatcccacgtgatggaccaggccatcaacaatgccatcaactacctgggggctgagtccctgcgcccattggtgcagacaccccccggtagctccgaggtggtgccagtcatcagctccatgtaccagctgcacaagcccccctcagatggccccccacggtccaaccattcagcacaggacgccgtggataacttgctgctgctgtccaaggccaagtctgtgtcatcggagcgagaggcctccccgagcaacagctgccaagactccacagatacagagagcaacgcggaggaacagcgcagcggccttatctacctaaccaaccacatcaacccgcatgcacgcaatgggctggctctcaaggaggagcagcgcgcctacgaggtgctgagggcggcctcagagaactcgcaggatgccttccgtgtggtcagcacgagtggcgagcagctgaaggtgtacaagtgcgaacactgccgcgtgctcttcctggatcacgtcatgtataccattcacatgggctgccatggctgccatggctttcgggatccctttgagtgtaacatgtgtggttatcacagccaggacaggtacgagttctcatcccatatcacgcggggggagcatcgttaccacctgagctaaacccagccaggccccactgaagcacaaagatagctggttatgcctccttcccggcagctggacccacagcggacaatgtgggagtggatttgcaggcagcatttgttcttttatgttggttgtttggcgtttcatttgcgttggaagataagtttttaatgttagtgacaggattgcattgcatcagcaacattcacaacatccatccttctagccagttttgttcactggtagctgaggtttcccggatatgtggcttcctaacactct

SEQ ID NO: 5 - DNA, 1170 bp, Mus musculus
  CDS (1)...(1170): mIk-4
atggatgtcgatgagggtcaagacatgtcccaagtttcaggaaaggagagccccccagtcagtgacactccagatgaaggggatgagcccatgcctgtccctgaggacctgtccactacctctggagcacagcagaactccaagagtgatcgaggcatgggtgaacggcctttccagtgcaaccagtctggggcctcctttacccagaaaggcaacctcctgcggcacatcaagctgcactcgggtgagaagcccttcaaatgccatctttgcaactatgcctgccgccggagggacgccctcaccggccacctgaggacgcactccgtcattaaggaagaaactaaccacaacgagatggcagaagacctgtgcaagataggagcagagaggtcccttgtcctggacaggctggcaagcaatgtcgccaaacgtaagagctctatgcctcagaaatttcttggagacaagtgcctgtcagacatgccctatgacagtgccaactatgagaaggaggatatgatgacatcccacgtgatggaccaggccatcaacaatgccatcaactacctgggggctgagtccctgcgcccattggtgcagacaccccccggtagctccgaggtggtgccagtcatcagctccatgtaccagctgcacaagcccccctcagatggccccccacggtccaaccattcagcacaggacgccgtggataacttgctgctgctgtccaaggccaagtctgtgtcatcggagcgagaggcctccccgagcaacagctgccaagactccacagatacagagagcaacgcggaggaacagcgcagcggccttatctacctaaccaaccacatcaacccgcatgcacgcaatgggctggctctcaaggaggagcagcgcgcctacgaggtgctgagggcggcctcagagaactcgcaggatgccttccgtgtggtcagcacgagtggcgagcagctgaaggtgtacaagtgcgaacactgccgcgtgctcttcctggatcacgtcatgtataccattcacatgggctgccatggctgccatggctttcgggatccctttgagtgtaacatgtgtggttatcacagccaggacaggtacgagttctcatcccatatcacgcggggggagcatcgttaccacctgagc

SEQ ID NO: 6 - DNA, 1128 bp, Mus musculus
  CDS (1)...(1128): mIk-5
atggatgtcgatgagggtcaagacatgtcccaagtttcaggaaaggagagccccccagtcagtgacactccagatgaaggggatgagcccatgcctgtccctgaggacctgtccactacctctggagcacagcagaactccaagagtgatcgaggcatggccagtaatgttaaagtagagactcagagtgatgaagagaatgggcgtgcctgtgaaatgaatggggaagaatgtgcagaggatttacgaatgcttgatgcctcgggagagaaaatgaatggctcccacagggaccaaggcagctcggctttgtcaggagttggaggcattcgacttcctaacggaaaactaaagtgtgatatctgtgggatcgtttgcatcgggcccaatgtgctcatggttcacaaaagaagtcatactggagacaagtgcctgtcagacatgccctatgacagtgccaactatgagaaggaggatatgatgacatcccacgtgatggaccaggccatcaacaatgccatcaactacctgggggctgagtccctgcgcccattggtgcagacaccccccggtagctccgaggtggtgccagtcatcagctccatgtaccagctgcacaagcccccctcagatggccccccacggtccaaccattcagcacaggacgccgtggataacttgctgctgctgtccaaggccaagtctgtgtcatcggagcgagaggcctccccgagcaacagctgccaagactccacagatacagagagcaacgcggaggaacagcgcagcggccttatctacctaaccaaccacatcaacccgcatgcacgcaatgggctggctctcaaggaggagcagcgcgcctacgaggtgctgagggcggcctcagagaactcgcaggatgccttccgtgtggtcagcacgagtggcgagcagctgaaggtgtacaagtgcgaacactgccgcgtgctcttcctggatcacgtcatgtataccattcacatgggctgccatggctgccatggctttcgggatccctttgagtgtaacatgtgtggttatcacagccaggacaggtacgagttctcatcccatatcacgcggggggagcatcgttaccacctgagc

SEQ ID NO: 7 - DNA, 1004 bp, Mus musculus
  CDS (1)...(1002)
ggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccgtcattaaagaagaaactaagcacagtgaaatggcagaagacctgtgcaagataggatcagagagatctctcgtgctggacagactagcaagtaatgtcgccaaacgtaagagctctatgcctcagaaatttcttggggacaagggcctgtccgacacgccctacgacagtgccacgtacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaggcgctcggagggcaccccgcgctccaaccactcggcccaggacagcgccgtggagtacctgctgctgctctccaaggccaagttggtgccctcggagcgcgaggcgtccccgagcaacagctgccaagactccacggacaccgagagcaacaacgaggagcagcgcagcggtcttatctacctgaccaaccacatcgcccgacgcgcgcaacgcgtgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagcta

SEQ ID NO: 8 - DNA, 103 bp, Mus musculus
tttggttataaatgtattgattgcatccccattacccagaaggccaatatttaattggagtcttaactcaattgtgttttcgtcagttggtaagcctcacaaa

SEQ ID NO: 9 - DNA, 116 bp, Mus musculus
atgggccttccgggcatgtacccaggtaagcactgaggccctgctgagctgcacccctccccctcccagcgcctgggccaggatggggctctgtggcctgtttcagccacaggagg

SEQ ID NO: 10 - DNA, 94 bp, Mus musculus
ccttgttgctgctgtgttgctatcttgtgacttatttttgcagtgacactgagtggcctcctgtgttgtctctttcagccagtaatgttaaagt

SEQ ID NO: 11 - DNA, 120 bp, Mus musculus
gagccctggcagatgtgtcctgtctgctgtgacactagaacaccattcaacccctgggtgtagatttcacttatgaccatctacttcccgcaggagacaagtgcctgtcagacatgccct

SEQ ID NO: 12 - DNA, 120 bp, Mus musculus
acatgtgtggttatcacagccaggacaggtacgagttctcatcccatatcacgcggggggagcatcgttaccacctgagctaaacccagccaggccccactgaagcacaaagatagctgg

SEQ ID NO: 13 - protein, 470 aa, Artificial Sequence
  consensus sequence
XXASNVKVETQSDEENGRACEMNGEECAEDLRMLDASGEKMNGSHRDQGSSALSGVGGIRLPNGKLKCDICGIXCIGPNVLMVHKRSHTGERPFQCNQCGASFTQKGNLLRHIKLHSGEKPFKCHLCNYACRRRDALTGHLRTHSVGKPHKCGYCGRSYKQRXSLEEHKERCHNYLESMGLPGXXXPVIKEETXHXEMAEDLCKIGXERSLVLDRLASNVAKRKSSMPQKFLGDKXLSDXPYDSAXYEKEXXMMXSHVMDXAINNAINYLGAESLRPLVQTPPGXSEVVPVISPMYQLHXXXSXGXPRSNHSAQDXAVXXLLLLSKAKXVXSEREASPSNSCQDSTDTESNXEEQRSGLIYLTNHIXXXAXXXXXLKEEXRAYXXLRAASENSQDAXRVVSTSGEQXKVYKCEHCRVLFLDHVMYTIHMXXXGCHGFRDPFECNMCGYHSQDRYEFSSHITRGEHRXHXS

SEQ ID NO: 14 - DNA, 38 bp, Artificial Sequence
  probe
agaagtttccataagatgatgaatgggggtggcagaga

SEQ ID NO: 15 - DNA, 24 bp, Artificial Sequence
  synthetically generated primer
ggctgccacggcttccgtgatcct

SEQ ID NO: 16 - DNA, 24 bp, Artificial Sequence
  synthetically generated primer
agcggtctggggaaacatctagga

SEQ ID NO: 17 - DNA, 24 bp, Artificial Sequence
  synthetically generated primer
agtaatgttaaagtagagactcag

SEQ ID NO: 18 - DNA, 24 bp, Artificial Sequence
  synthetically generated primer
gtatgacttcttttgtgaaccatg

SEQ ID NO: 19 - DNA, 24 bp, Artificial Sequence
  synthetically generated primer
ccagcctctgagcccagaaagcga

SEQ ID NO: 20 - DNA, 24 bp, Artificial Sequence
  synthetically generated primer
cactacctctggagcacagcagaa

SEQ ID NO: 21 - DNA, 21 bp, Artificial Sequence
  synthetically generated primer
ggtgaacggcctttccagtgc

SEQ ID NO: 22 - DNA, 21 bp, Artificial Sequence
  synthetically generated primer
tctgaggcatagagctcttac

SEQ ID NO: 23 - DNA, 24 bp, Artificial Sequence
  synthetically generated primer
catagggcatgtctgacaggcact

SEQ ID NO: 24 - DNA, 28 bp, Artificial Sequence
  synthetically generated primer
tcagcttttgggaatgtattccctgtca

SEQ ID NO: 25 - DNA, 24 bp, Artificial Sequence
  synthetically generated primer
tcagcttttgagaataccctgtca

SEQ ID NO: 26 - DNA, 17 bp, Artificial Sequence
  synthetically generated primer
ggcatgactcagagcga

SEQ ID NO: 27 - DNA, 25 bp, Artificial Sequence
  synthetically generated primer
ccttcatctggagtgtcactgactg

SEQ ID NO: 28 - DNA, 22 bp, Artificial Sequence
  synthetically generated primer
ctgaaacttgggacatgtcttg

SEQ ID NO: 29 - DNA, 30 bp, Artificial Sequence
  synthetically generated primer
aaaggatccgaacataactatggatcagcc

SEQ ID NO: 30 - DNA, 29 bp, Artificial Sequence
  synthetically generated primer
tttaccggtgtcttcaggttatctcctgc

SEQ ID NO: 31 - DNA, 19 bp, Artificial Sequence
  synthetically generated primer
cgtaaaggccacaagttca

SEQ ID NO: 32 - DNA, 20 bp, Artificial Sequence
  synthetically generated primer
cttgaagttcaccttgatgc

SEQ ID NO: 33 - DNA, 62 bp, Artificial Sequence
  synthetically generated primer
tcgacgatcgatcgatcgatcataacttcgtataatgtatgctatacgaagttattaagctt

SEQ ID NO: 34 - DNA, 41 bp, Artificial Sequence
  synthetically generated primer
gatccataacttcgtataatgtatgctatacgaagttattt

SEQ ID NO: 35 - DNA, 46 bp, Artificial Sequence
  synthetically generated primer
ctagaaataacttcgtatagcatacattatacgaagttatggatcc

SEQ ID NO: 36 - protein, 21 aa, Artificial Sequence
  exemplary motif
CXXCXXXXXXXXXXXXHXXXH

SEQ ID NO: 37 - protein, 431 aa, Mus musculus
MDVDEGQDMSQVSGKESPPVSDTPDEGDEPMPVPEDLSTTSGAQQNSKSDRGMGQRPFQCNQSGASFTQKGNLLRHIKLHSGEKPFKCHLCNYACRRRDALTGHLRTHSVGKPHKCGYCGRSYKQRSSLEEHKERCHNYLESMGLPGVCPVIKEETNHNEMAEDLCKIGAERSLVLDRLASNVAKRKSSMPQKFLGDKCLSDMPYDSANYEKEDMMTSHVMDQAINNAINYLGAESLRPLVQTPPGSSEVVPVISSMYQLHKPPSDGPPRSNHSAQDAVDNLLLLSKAKSVSSEREASPSNSCQDSTDTESNAEEQRSGLIYLTNHINPHARNGLALKEEQRAYEVLRAASENSQDAFRVVSTSGEQLKVYKCEHCRVLFLDHVMYTIHMGCHGCHGFRDPFECNMCGYHSQDRYEFSSHITRGEHRYHLS

SEQ ID NO: 38 - protein, 461 aa, Homo sapiens
NVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRTSLEEHKERCHNYLESMGLPGTLYPVIKEETKHSEMAEDLCKIGSERSLVLDRLASNVAKRKSSMPQKFLGDKGLSDTPYDSATYEKENEMMKSHVMDQAINNAINYLGAESLRPLVQTPPGGSEVVPVISPMYQLHRRSEGTPRSNHSAQDSAVEYLLLLSKAKLVPSEREASPSNSCQDSTDTESNNEEQRSGLIYLTNHIARRAQRVSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 39 - protein, 432 aa, Mus musculus
MDVDEGQDMSQVSGKESPPVSDTPDEGDEPMPVPEDLSTTSGAQQNSKSDRGMASNVKVETQSDEENGRACEMNGEECAEDLRMLDASGEKMNGSHRDQGSSALSGVGGIRLPNGKLKCDICGIVCIGPNVLMVHKRSHTGERPFQCNQSGASFTQKGNLLRHIKLHSGEKPFKCHLCNYACRRRDALTGHLRTHSGDKCLSDMPYDSANYEKEDMMTSHVMDQAINNAINYLGAESLRPLVQTPPGSSEVVPVISSMYQLHKPPSDGPPRSNHSAQDAVDNLLLLSKAKSVSSEREASPSNSCQDSTDTESNAEEQRSGLIYLTNHINPHARNGLALKEEQRAYEVLRAASENSQDAFRVVSTSGEQLKVYKCEHCRVLFLDHVMYTIHMGCHGCHGFRDPFECNMCGYHSQDRYEFSSHITRGEHRYHLS

SEQ ID NO: 40 - protein, 518 aa, Mus musculus
MDVDEGQDMSQVSGKESPPVSDTPDEGDEPMPVPEDLSTTSGAQQNSKSDRGMASNVKVETQSDEENGRACEMNGEECAEDLRMLDASGEKMNGSHRDQGSSALSGVGGIRLPNGKLKCDICGIVCIGPNVLMVHKRSHTGERPFQCNQSGASFTQKGNLLRHIKLHSGEKPFKCHLCNYACRRRDALTGHLRTHSVGKPHKCGYCGRSYKQRSSLEEHKERCHNYLESMGLPGVCPVIKEETNHNEMAEDLCKIGAERSLVLDRLASNVAKRKSSMPQKFLGDKCLSDMPYDSANYEKEDMMTSHVMDQAINNAINYLGAESLRPLVQTPPGSSEVVPVISSMYQLHKPPSDGPPRSNHSAQDAVDNLLLLSKAKSVSSEREASPSNSCQDSTDTESNAEEQRSGLIYLTNHINPHARNGLALKEEQRAYEVLRAASENSQDAFRVVSTSGEQLKVYKCEHCRVLFLDHVMYTIHMGCHGCHGFRDPFECNMCGYHSQDRYEFSSHITRGEHRYHLS

SEQ ID NO: 41 - protein, 390 aa, Mus musculus
MDVDEGQDMSQVSGKESPPVSDTPDEGDEPMPVPEDLSTTSGAQQNSKSDRGMGERPFQCNQSGASFTQKGNLLRHIKLHSGEKPFKCHLCNYACRRRDALTGHLRTHSVIKEETNHNEMAEDLCKIGAERSLVLDRLASNVAKRKSSMPQKFLGDKCLSDMPYDSANYEKEDMMTSHVMDQAINNAINYLGAESLRPLVQTPPGSSEVVPVISSMYQLHKPPSDGPPRSNHSAQDAVDNLLLLSKAKSVSSEREASPSNSCQDSTDTESNAEEQRSGLIYLTNHINPHARNGLALKEEQRAYEVLRAASENSQDAFRVVSTSGEQLKVYKCEHCRVLFLDHVMYTIHMGCHGCHGFRDPFECNMCGYHSQDRYEFSSHITRGEHRYHLS

SEQ ID NO: 42 - protein, 376 aa, Mus musculus
MDVDEGQDMSQVSGKESPPVSDTPDEGDEPMPVPEDLSTTSGAQQNSKSDRGMASNVKVETQSDEENGRACEMNGEECAEDLRMLDASGEKMNGSHRDQGSSALSGVGGIRLPNGKLKCDICGIVCIGPNVLMVHKRSHTGDKCLSDMPYDSANYEKEDMMTSHVMDQAINNAINYLGAESLRPLVQTPPGSSEVVPVISSMYQLHKPPSDGPPRSNHSAQDAVDNLLLLSKAKSVSSEREASPSNSCQDSTDTESNAEEQRSGLIYLTNHINPHARNGLALKEEQRAYEVLRAASENSQDAFRVVSTSGEQLKVYKCEHCRVLFLDHVMYTIHMGCHGCHGFRDPFECNMCGYHSQDRYEFSSHITRGEHRYHLS

SEQ ID NO: 43 - protein, 334 aa, Mus musculus
GERPFQCNQCGASFTQKGNLLRHIKLHSGEKPFKCHLCNYACRRRDALTGHLRTHSVIKEETKHSEMAEDLCKIGSERSLVLDRLASNVAKRKSSMPQKFLGDKGLSDTPYDSATYEKENEMMKSHVMDQAINNAINYLGAESLRPLVQTPPGGSEVVPVISPMYQLHRRSEGTPRSNHSAQDSAVEYLLLLSKAKLVPSEREASPSNSCQDSTDTESNNEEQRSGLIYLTNHIARRAQRVSLKEEHRAYDLLRAASENSQDALRVVSTSGEQMKVYKCEHCRVLFLDHVMYTIHMGCHGFRDPFECNMCGYHSQDRYEFSSHITRGEHRFHMS

(19) United States
(12) Patent Application Publication
(10) Pub. No.: US 2002/0104112 A1
(43) Pub. Date: Aug. 1, 2002

(54) Ikaros regulatory elements and uses thereof

(76) Inventor: Katia Georgopoulos, Lexington, MA (US)

Correspondence Address: LOUIS MYERS, FISH & RICHARDSON P.C., 225 Franklin Street, Boston, MA, 02110-2804, US

(21) Appl. No.: 09/755,830

(22) Filed: Jan. 5, 2001

Related U.S. Application Data
(63) Continuation-in-part of application No. 08/283,300, filed on Jul. 29, 1994.

(51) Int. Cl. A01K 67/027; C07H 21/04; C12N 9/00; C12N 15/74

(52) U.S. Cl. 800/18; 435/183; 435/320.1; 536/23.1

(57) ABSTRACT

The invention features Ikaros transcriptional control regions which include one or more Ikaros regulatory elements. Such regulatory regions can be used, for example, to direct expression of a sequence functionally unrelated to the Ikaros gene, e.g., a sequence encoding a reporter molecule. The invention further features a transgenic animal having an Ikaros transgene which includes an Ikaros transcriptional control region and a sequence functionally unrelated to Ikaros, e.g., a sequence encoding a reporter molecule, and methods of using such transgenic animals to evaluate hematopoietic development of an immune system component.

This application is a continuation-in-part of U.S. Ser. No. 08/283,300, filed Jul. 29, 1994 which is a continuation-in-part of U.S. Ser. No. 08/238,212, filed May 2, 1994, U.S. Ser. No. 08/121,438, filed Sep. 14, 1993, and U.S. Ser. No. 07/946,233, filed Sep. 14, 1992, all of which are hereby incorporated by reference.

### BACKGROUND OF THE INVENTION

The generation of the T cell repertoire from a progenitor stem cell proceeds through a differentiation pathway. All blood cells originate from a hematopoietic stem cell. This population of stem cells can self renew or become pluripotent stem cells. Such pluripotent stem cells can become committed to differentiate along particular lineages. For example, pluripotent stem cells can give rise to either lymphoid progenitor cells or myeloid progenitor cells. Such lymphoid progenitor can in turn give rise to either B-lymphocytes or T-lymphocytes. Myeloid progenitor cells can become committed to differentiate into, for example, erthyroid, megakaryocyte, granulocytic or monocytic lineages.

In the differentiation pathway, the later intrathymic steps are well documented while the early extrathymic events are only poorly characterized. One of the earliest definitive T cell differentiation markers is the CD3δ gene of the CD3/TCR complex.

### SUMMARY OF THE INVENTION

The Ikaros locus is a master regulatory locus which is intricately intertwined with the regulation of hematopoietic development. The Ikaros locus is also expressed in certain nervous tissue and is active in the regulation of the cell cycle. It is active at various times in development and exerts an extremely pleiotropic hematopoietic development phenotype. For example, the Ikaros gene is characterized by a complex and striking pattern of expression in terms of tissue-specificity, is temporally regulated, and is regulated in terms of the profile of isoform expression. All of these observations are consistent with a gene which provides critical developmental control at a number of points in development. The phenotypes of Ikaros transgenic animals of the invention confirm the fundamental and multifaceted role of the Ikaros gene. For example, mice which are heterozygotic for a deletion of portions of exons 3 and 4 (which encode a region involved in DNA binding), develop extremely aggressive lymphomas. Initial data suggest that human lymphoma tissue often exhibit chromosomal aberrations involving Ikaros. Homozygotes for the exon 3/4 deletion are poorly viable. Transgenic mice with a different deletion, a deletion of exon 7 (which is believed to be active in activation and dimerization of the Ikaros gene product) exhibits a very different phenotype. Mice which are heterozygous for an exon 7 deletion are healthy. Mice which are homozygous for an exon 7 deletion have no B cells, no NK cells, and no γδ T cells. While T cells are present, the populations of CD4⁺/CD8⁺, CD4⁺/CD8⁻, and CD4⁻/CD8⁺ are skewed (the proportion of CD4⁺/CD8⁺ cells is decreased relative to wild type, the proportion of CD4⁺/CD8⁻ cells is increased relative to wild type, and the proportion of CD4⁻/CD8⁺ cells is unchanged relative to wild type). It has also been found that Ikaros regulatory elements play an important role in directing hematopoietic development. Depending on which regulatory element, or combination of regulatory elements, is involved in transcription, progression along various differentiation pathways of the hematopoietic lineage can occur. For example, involvement of different Ikaros promoter elements can result in directed expression of B-cells, neutrophils or both. In addition, involvement of various Ikaros enhancer elements and/or insulator elements can result in, for example, directed expression of T-cells.

The central and multifaceted role of Ikaros in development, and the variety of phenotypes exhibited by Ikaros transgenic animals and cells, render Ikaros transgenic animals and cells useful, e.g., in a variety of assays, screens, and other methods. For example, animals, cells and methods of the invention can be used to elucidate and characterize the function of the immune system, mechanisms of development, ways in which components of the immune system interact, ways in which the cell cycle is regulated, mechanisms of immune tolerance, and mechanisms of the development of immune or nervous tissue disorders. The cells, animals, and methods of the invention are also useful, e.g., for evaluating or discovering treatments which can be used to treat immune or nervous tissue disorders, for discovering or for evaluating treatments or methods of inducing immunological tolerance, e.g., to transplanted tissues. By way of example, Ikaros mice which develop lymphomas are useful not only for investigating the molecular basis of these disorders but also for screening treatments for the ability to treat such disorders. Ikaros mice which lack one or more components of the immune system are useful in a variety of reconstitution experiments.

Accordingly, the invention features, a transgenic animal, e.g., a mammal, e.g., preferably a nonhuman primate or a rodent, e.g., a mouse, having an Ikaros transgene. In other preferred embodiments, the transgenic animal is a fish, e.g., a zebrafish; a nemaotde, e.g., caenorhabditis elegans; an amphibian, e.g., a frog or an axolotl.

In a preferred embodiment, the animal is a transgenic animal, e.g., a transgenic mouse, having a transgene which includes an Ikaros transcriptional control region and a sequence encoding a protein functionally unrelated to Ikaros, e.g., a sequence encoding a reporter molecule.

In preferred embodiments, the animal further includes a mutated Ikaros transgene, the mutation occurring in, or altering, e.g., a domain of the Ikaros gene described herein. The transgenic animal or cell can: be heterozygous for an Ikaros transgene, e.g., a mutated Ikaros transgene; be homozygous for an Ikaros transgene, e.g., a mutated Ikaros transgene; include a first Ikaros transgene, e.g., a transgene which includes an Ikaros transcriptional control region and a sequence encoding an unrelated protein, and a second Ikaros transgene, e.g., a mutated Ikaros transgene; include an Ikaros transgene, e.g., a transgene which includes an Ikaros transcriptional control region and a sequence encoding an unrelated protein, and a second transgene which is other than an Ikaros transgene, e.g., another protein involved in hematopoiesis, e.g., an Aiolos transgene and/or a Helios transgene, e.g., a mutated Aiolos and/or Helios transgene.

In another aspect, the invention features a method of evaluating a component or a cell lineage, e.g., for evaluating development of a component or cell lineage of the immune system, e.g., the development of a hematopoietic cell or cells of the immune system. The method includes providing a transgenic animal, or cell or tissue therefrom, having an Ikaros transgene which includes an Ikaros transcriptional control region and a sequence encoding a protein functionally unrelated to Ikaros, e.g., a sequence encoding a reporter molecule, and monitoring expression of the protein unrelated to Ikaros, e.g., monitoring expression of the reporter molecule. Preferably, the Ikaros transcriptional control region includes one or more regulatory element(s) of Ikaros which directs expression of the immune component of interest. Types of development which can be evaluated include, e.g., the ontogeny of a component or cell lineage of the immune system, activation of a component or cell lineage of the immune system, the migration of a component or cell lineage of the immune system, regions of action of a component or cell lineage of the immune system and ways in which components of the immune system interact. Examples of immune system components which can be evaluated include hematopoietic cells and cell lineages, e.g., hematopoietic stem cells, multipotent progenitors, oligopotent progenitors (e.g., lymphoid or myeloid progenitors), cells committed to the B-cell lineage, cells committed to the T-cell lineage, cells committed to a myeloid cell lineage (e.g., granulocyte monocyte CFU cells), T-lymphocytes, B-lymphocytes, NK cells, and neutrophils.

Development of a component or components of the immune system can be evaluated in a living animal, a dead animal, or a tissue taken from a live or dead animal. In a preferred embodiment, the protein unrelated to Ikaros is a reporter molecule, e.g., a colored or fluorescent molecule, and the immune system component is monitored on the live animal. Preferably, the method includes detecting a signal, e.g., a fluorescent signal, on the live animal, e.g., using a confocal microscope in order to monitor expression of the immune system component.

In another aspect, the invention features a method for evaluating the effect of a treatment on a transgenic cell or animal having an Ikaros transgene, e.g., the effect of the treatment on the development of the immune system. The method includes administering the treatment to a cell or animal having an Ikaros transgene, and evaluating the effect of the treatment on the cell or animal. Preferably, the Ikaros transgene includes an Ikaros transcriptional control region and a sequence functionally unrelated to Ikaros, e.g., a sequence encoding a reporter molecule. The effect can be, e.g., the effect of the treatment on: the immune system or a component thereof, the nervous system or a component thereof, or the cell cycle. Immune system effects include e.g., T cell activation, T cell development, the ability to mount an immune response, the ability to give rise to a component of the immune system, B cell development, NK cell development, myeloid cell development, or the ratios CD4⁺/CD8⁺, CD4⁺/CD8⁻ and CD4⁻/CD8⁺.

In preferred embodiments the treatment can include: the administration of a drug, chemical, or other substance; the administration of ionizing radiation; the administration of an antibody, e.g., an antibody directed against a molecule or cell of the immune system; administration of a substance or other treatment which suppresses the immune system; or administration of a substance or other treatment which activates or boosts the function of the immune system; introduction of a nucleic acid, e.g., a nucleic acid which encodes or expresses a gene product, e.g., a component of the immune system; the introduction of a protein, e.g., a protein which is a component of the immune system.

In another aspect, the invention features a method for evaluating the effect of a treatment on an immune system component. The method includes: (1) supplying a transgenic cell or animal having an Ikaros transgene; (2) supplying the immune system component; (3) administering the treatment; and (4) evaluating the effect of the treatment on the immune system component.

In yet another aspect, the invention features a method for evaluating the interaction of a first immune system component with a second immune system component. The method includes: (1) supplying a transgenic cell or animal, e.g., a mammal, having an Ikaros transgene; (2) introducing the first and second immune system component into the transgenic cell or mammal; and (3) evaluating an interaction between the first and second immune system components.

Mice with mutant Ikaros transgenes which eliminate many of the normal components of the immune system, e.g., mice homozygous for a transgene having a deletion for some or all of exon 7, are particularly useful for “reconstitution experiments.”

Ikaros transgenic mice which exhibit a phenotype characteristic of an immune system disorder, e.g., mice which are homozygous for a transgene having a deletion of all or some of exons 3 and 4, can serve as model systems for human disorders, e.g., for lymphoma.

In another aspect, the invention features a method for evaluating the effect of a treatment on an immune system disorder, e.g., a neoplastic disorder, a lymphoma, a T cell related lymphoma, including: administering the treatment to a cell or animal having an Ikaros transgene, and evaluating the effect of the treatment on the cell or animal.

In another aspect, the invention features, a method for evaluating the effect of a treatment on the nervous system comprising administering the treatment to a transgenic cell or an animal having an Ikaros transgene, and evaluating the effect of the treatment on the cell or the animal.

In another aspect, the invention features, a method for evaluating the effect of a treatment on a disorder of the nervous system, e.g., neurodegenerative disorder, e.g., Alzheimer's disease, Huntington's disease, Parkinson's disease, e.g., a neuroactive substance, e.g., neurotransmitter, imbalance, including administering the treatment to a cell or animal having an Ikaros transgene, and evaluating the effect of the treatment on the cell or animal.

In another aspect, the invention features an Ikaros transcriptional control region which includes an Ikaros regulatory element or combinations of Ikaros regulatory elements. In a preferred embodiment, the regulatory element can be one or more of Ikaros promoter(s), enhancer(s) and/or insulator sequence(s). The regulatory elements can be 5′ regulatory elements, intronic elements, and/or 3′ regulatory elements of Ikaros. In a preferred embodiment, when there is a combination of Ikaros regulatory elements, the complement or placement of the regulatory elements can differ from where it is naturally found in the Ikaros gene. In a preferred embodiment, a DNase I HSS cluster of Ikaros includes the regulatory element and all or a portion of the DNase I HSS cluster is included in the transcriptional control region. In a preferred embodiment, the Ikaros transcriptional control region includes: at least a portion of the β cluster containing a promoter, e.g., an R19 promoter, and/or at least a portion of the γ cluster containing a promoter, e.g., an R10 promoter. In other embodiments, the Ikaros transcriptional control region can include one or more promoter(s), e.g., a promoter from the β cluster and/or the γ cluster, and one or more Ikaros regulatory element(s), e.g., one or more Ikaros regulatory element from the α cluster, the ε cluster, the η cluster and/or the θ cluster. For example, the Ikaros transcriptional control region can include the γ cluster or a promoter-containing portion thereof and the ε cluster or a portion thereof. In other embodiments, the Ikaros transgene can include all or a promoter-containing portion of the β cluster and/or all or a promoter-containing portion from the γ cluster and: all or a portion of the α cluster; all or a portion of the δ cluster; all or a portion of the ε cluster; all or a portion of the ζ cluster; all or a portion of the η cluster; all or a portion of the θ cluster; combinations of two, three, four, or five of the α cluster, the δ cluster, the ε cluster, the ζ cluster, the η cluster, the θ cluster, or portions thereof; all of the α cluster, the δ cluster, the ε cluster, the ζ cluster, the η cluster and the θ cluster, or portions thereof.

In another aspect, the invention features a DNA construct which includes an Ikaros transcriptional control region, as described herein, and a sequence encoding a protein or polypeptide. In a preferred embodiment, the sequence can encode an Ikaros protein or a variant thereof as described herein. In a preferred embodiment, when the sequence encodes Ikaros or a variant thereof, the Ikaros transcriptional control region preferably includes one or more Ikaros regulatory element(s) but not all of the Ikaros regulatory elements described herein. In another preferred embodiment, the sequence encodes a protein or polypeptide functionally unrelated to Ikaros, e.g., the sequence encodes a reporter molecule. When the sequence encodes a protein unrelated to Ikaros, e.g., a reporter molecule, the Ikaros transcriptional control region can include one, two, three, four, five, six, seven or all of the Ikaros regulatory elements described herein. Preferably, when there is a combination of Ikaros regulatory elements, the complement or placement of the regulatory elements can differ from where it is naturally found in the Ikaros gene. For example, an element: which is normally 5′, can be 5′, 3′ or intronic with regard to the sequence encoding a protein or polypeptide, e.g., a reporter molecule; which is normally 3′ can be 5′, 3′ or intronic with regard to the sequence encoding a protein or polypeptide, e.g., a reporter molecule; which is intronic can be 5′, 3′ or intronic with regard to the sequence encoding a protein or polypeptide, e.g., a reporter molecule.

The Ikaros gene is active in the early differentiation of lymphocytes, e.g. T cells and B cells. The gene encodes a family of unique zinc finger proteins, the Ikaros proteins. The proteins of the Ikaros family are isoforms which arise from differential splicing of Ikaros gene transcripts. The isoforms of the Ikaros family generally include a common 3′ exon (Ikaros exon E7, which includes amino acid residues 283-518 of the mouse Ikaros protein represented by SEQ ID NO:4, and amino acid residues 229-461 of the human Ikaros protein represented by SEQ ID NO:2) but differ in the 5′ region. The Ikaros family includes all naturally occurring splicing variants which arise from transcription and processing of the Ikaros gene. Five such isoforms are described in copending U.S. patent application Ser. No. 08/121,438, filed Sep. 14, 1993. The Ikaros family also includes other isoforms, including those generated by mutagenesis and/or by in vitro exon shuffling. The naturally occurring Ikaros proteins can bind and activate (to differing extents) the enhancer of the CD3 δ gene, and are expressed primarily if not solely in T cells in the adult. The expression pattern of this transcription factor during embryonic development show that Ikaros proteins play a role as a genetic switch regulating entry into the T cell lineage. The Ikaros gene is also expressed in the proximal corpus striatum during early embryogenesis in mice.

As described above, the Ikaros gene is a master regulator for lymphocyte specification. The Ikaros gene was initially described for its ability to mediate the activity of an enhancer element in the CD3 3δ gene, an early and definitive marker of the T cell differentiation (Georgopoulos, K. et al. (1992) Science 258:808). During embryogenesis, Ikaros expression is restricted to sites of hemopoiesis where it precedes and overlaps with areas of lymphocyte differentiation. Ikaros is expressed in early B cells and in T cells and their progenitors in the adult organism. Consistent with its role as a master regulator of lymphocyte specific gene expression, the Ikaros gene encodes a family of zinc finger DNA binding proteins by means of differential splicing (Molnar et al., 1994). These protein isoforms display overlapping but distinct DNA binding specificities and range from strong activators to suppressors of transcription. Together, Ikaros proteins appear to control multiple layers of gene expression during lymphocyte ontogeny in the embryo and in the adult. Significantly, high affinity binding sites for the Ikaros proteins were identified in the regulatory domains of many lymphocyte specific genes among which are the members of the CD3/TCR complex, terminal deoxyribonucleotidyl transferase (TdT), the IL-2 receptor, immunoglobulin heavy and light chains and the signal transducing molecule Igα. These genes are all important components in T and B cell differentiation pathways and their expression is a prerequisite for lymphocyte development. In addition, the Ikaros proteins can bind and activate a subset of NF-KB sites implicated in stimulating gene expression in the activated T cell (Beg, A. A. and Baldwin, A. S. J. (1993) Genes Dev. 7:2064-2070; Lenardo, M. J. and Baltimore, D. (1989) Cell 58:227-229). The Ikaros gene and its splicing products are highly conserved between mice and man, in further support of a master switch function for the lymphopoietic system across species (Molnar, et al., 1994).

A small number of regulatory genes have been described which control cell fate decisions at specific stages of the hemo-lymphoid pathway (Sieweke et al. (1998) Curr. Opin. Genet. Dev. 8(5):545-551; Georgopoulos (1997) Curr. Opin. Immunology 9(2):222-227). Of these regulators, Ikaros encodes a family of zinc finger transcription factors which are critical for progression through a number of branch points of this developmental pathway. Georgopoulos (1997) Curr. Opin. Immunology 9(2):222-227. Mice with an inactivating mutation in the Ikaros gene, display a reduction in hematopoietic stem cell (HSC) activity in both the fetus and in the adult, indicating that either the production of HSC from a mesodermal precursor or its self-renewal properties are impaired. Nichogiannopoulou et al. (1999) J. Exp. Med. 190(9):1201-1214. Significantly, Ikaros null mice lack all B-lymphocytes from the earliest described precursors in the fetal liver and in the bone marrow to the mature populations present in peripheral lymphatic centers and in the peritoneum. Wang et al. (1996) Immunity 5(6):537-549. Cells of the fetal T-lineages are also absent and only a small number of T cell precursors is detected in the thymus after birth. Wang et al. (1996) Immunity 5(6):537-549. In sharp contrast to the severe impairment in the production of B and T cell precursors, there is an increase in myeloid and erythroid precursors in Ikaros null mice. CFU-Multi and CPU-GM are significantly elevated, especially relative to the decrease manifested in the HSC compartment and myelocytes are abundantly present in the bone marrow and spleen of the mutant mice. Nichogiannopoulou et al. (1999) J. Exp. Med. 190(9):1201-1214. Mac-1⁺ cells of a Gr-1^{h1} phenotype are absent although plenty of cells with a neutrophil morphology are detected in these sites indicating a potential deregulation of the Ly6G gene encoding Gr-1. Thus, Ikaros expression is not only important for production and possibly maintenance of the HSC, but also for its regulated differentiation along the lymphoid and myeloid pathways.

Ikaros plays also a critical role during T cell differentiation. The small number of postnatal T cell precursors detected in the thymus of Ikaros null mice CM progress to the double positive and positive CD4⁺ single stage of differentiation in the absence of pre-TCR signaling. Winandy et al. (1999) J. Exp. Med. 190(8):1039-1048. In the presence of TCR signaling, a relative increase in the number of CD4⁺/TCR⁺ thymocytes is detected which is accompanied by a decrease in double positives but not in CD8⁺TCR⁺ cells. Wang et al. (1996) Immunity 5(6):537-549. In their majority, these CD4⁺/TCR⁺ cells are not properly selected and do not exit to the periphery. In mice heterozygous for the Ikaros null or dominant negative mutations, T cell populations do not appear to be developmentally abnormal, however, when stimulated in vitro through the T cell receptor they display augmented proliferative responses and in vivo undergo transformation to a neoplastic stage. Avitahl et al. (1999) Immunity 10(3):333-343.

The phenotypes manifested in the Ikaros deficient mice are in accordance with its expression in the hemo-lymphoid system. In the developing embryo, Ikaros mRNA is seen at early sites of hemopoiesis; in ES blood islands of the yolk sac, in a small number of mesodermal cells within the embryo proper (T. Ikeda, unpublished results), and in the fetal liver from E9.5. Ikaros is expressed in the fetal thymus from E10.5 at the onset of its population with fetal lymphoid precursors. Georgopoulos, K. et al. (1992) Science 258:808). In the bone marrow, Ikaros is expressed in a population enriched for the pluripotent and self-renewing HSC (lin⁻/ Scal⁻/ckit⁺), and continues to be expressed along a precursor population (lin⁻/ Scal⁻/ckit⁺) enriched in myeloid potential. Morgan et al. (1997) EMBO J 16(8):2004-2013; Kelley et al. (1998) Curr. Biol. 8(9):508-515. Upon differentiation to monocytes, macrophages and erythrocytes, Ikaros expression is down regulated, however, it is maintained at significant levels in neutrophils. Klug et al. (1998) Proc. Natl Acad. Sci. USA 95(2):657-662. In contrast, Ikaros is upregulated from the early thymocyte precursors (DN) to differentiating (DP) thymocytes and is expressed in mature (SP) T cells in the fetus and in the adult. In a similar fashion, it is upregulated during differentiation from the pro-B to the pre-B cell stage. Georgopoulos (1997) Curr. Opin. Immunology 9(2):222-227. Among the hemo-lymphoid populations, Ikaros expression is highest in double positive thymocytes and mature T cells, populations that display strong haplo-insufficiency phenotypes in mice heterozygous for the Ikaros mutations.

Thus, proper regulation of Ikaros expression is critical for progression and homeostasis along multiple differentiation pathways in the hemo-lymphoid system. To identify the transcriptional regulatory elements involved, the mouse Ikaros locus was mapped over a region of approximately 120 kB and eight distinct clusters of lymphoid specific DNaseI HSS were identified. Two distinct 5′ untranslated mRNA ends were identified by 5′ RACE and primer extension and the encoding exons were mapped in the vicinity of two clusters of lymphoid-specific DNaseI HSS. Regions containing the two clusters and the associated promoters were tested for activity in transgenic mice. The two promoter regions, referred to herein as R10 and R19, directed expression in B cells and neutrophils or in neutrophils only. The R10 promoter region in conjunction with an intronic DNaseI HSS cluster gained high levels of activity in differentiating and mature T cells. Finally, the B cell specific elements that reside in the R10 promoter region appear to be amenable to negative auto regulation.

Other features and advantages of the invention will be apparent from the following description and from the claims.

### DETAILED DESCRIPTION OF THE INVENTION

The drawings are first briefly described.

### DRAWINGS

FIG. 1 is a map of the DNA sequence of a murine Ikaros cDNA and the desired amino acid sequence encoded thereby (SEQ ID NO:1).

FIG. 2 is a partial sequence of a human Ikaros cDNA (SEQ ID NO:2).

FIG. 3 is a depiction of the partial amino acid composition of the IK-1 cDNA, including Ex3, Ex4, Ex5, Ex6, and Ex7 (SEQ ID NO:4).

FIG. 4 is a diagram of exon usage in the Ikaros 1-5 cDNAs. Exon numbers are indicated at the bottom left hand corner of each box (Ex). Zinc finger modules are shown on top of the encoding exons (Fx).

FIG. 5 is a depiction of the exon organization at the Ikaros locus indicating primer sets 1/2 and 3/4 used for amplification of the respective isoforms.

FIG. 6 is a map of the genomic organization of the mouse Ikaros gene. Intronic or uncharacterized DNA is indicated as a line between 5′ and 3′. Exons are indicated as boxes. Lines numbered f2, f10, f4, and f8 indicate phage inserts corresponding to the sequence immediately above. Restriction sites are indicated by the usual abbreviations.

FIG. 7 is a schematic of an Ikaros view of the hemopoietic system which shows Ikaros expression and its putative roles in differentiation

FIG. 8A is a map of the genomic organization of the mouse Ikaros gene. The entire gene is approximately 120 kb in length. Intronic or untranslated DNA is indicated as a line between 5′ and 3′. Exons are indicated as solid boxes labeled Ex1, Ex2, Ex3, 4, 5, 6, and 7. The R19 and R10 promoters are indicated by open boxes labeled R19 and R10. FIG. 8B depicts the strategy for analysis of the 5′ end of Ikaros mRNA by 5′ rapid amplification of the cDNA ends and primer extension using primers from exons 1 and 2.

FIG. 9A is a map of the mouse Ikaros gene. Exons are indicated as solid boxes. The R19 and R10 promoters are indicated by open boxes. DNaseI HSS are indicated by arrows, solid black arrows ▾ designate the DNaseI HSS with specificity for the thymus, open arrows ∇ designate the DNaseI HSS with specificity for the spleen and partially solid arrows [symbol] designate DNaseI HSS with specificity for both the thymus and spleen. The DNaseI HSS clusters are labeled α, γ, δ, ε, ζ, η and θ. FIG. 9B shows the results of Southern blot analysis of DNA which was obtained from nuclei of the thymus, spleen and liver that have been digested with increasing amounts of DNaseI, purified and digested with restriction enzymes.

FIG. 10A is a map of the regions of mouse Ikaros which includes the β DNaseI HSS cluster (including the R19 promoter), the γ DNaseI HSS cluster (which includes the R10 promoter) and a portion of the ε DNaseI HSS cluster. Solid arrows indicate a DNaseI HSS, open boxes indicate the R19 and the R10 promoters. Exon 1 is indicated by a solid box (Ex1). FIG. 10B depicts various Ikaros regulatory elements which were used for expression of green fluorescent protein (GFP). The open boxes indicate either the RI 9 or the R10 promoter. The vertical black line indicates an Exon 1 splice acceptor (with a mutate ATG). The solid box indicates the sequence encoding EGFP (the open box at the end indicates a polyA site). The arrows indicate IαxP sites and the thicker line indicates a portion of the ε DNaseI HSS cluster which includes T1 (thymus) and TS2 (thymus and spleen) DNase HSS site.

FIG. 11 depicts GFP expression in the bone marrow of transgenic mice in which the sequence encoding GFP is either under control of the R19 promoter (R19-GFP) or the R10 promoter (R10-GFP). The bone marrow was stained with lineage specific promoters (Mac-1+, and Gr-1+ are indicative of neutrophils; B220+ is indicative of B cells).

FIG. 12 depicts GFP expression in the spleen of transgenic mice in which the sequence encoding GFP is either under control of the R19 promoter (R19-GFP) or the R10 promoter (R10-GFP). The spleen was stained with lineage specific promoters (Mac-1+, and Gr-1+ are indicative of neutrophils; B220+ is indicative of B cells; CD4, CD8 can be indicative of T cells).

FIG. 13A demonstrates the correlation of CD44 and/or CD25 expression and various stages of T cell development. The percentages provide the percentage of each cell type seen when the transgene includes the R10 promoter and a portion of the ε DNaseI HSS cluster. FIGS. 13B and 13C depict GFP expression in the spleen of transgenic mice in which the sequence encoding GFP is either under control of the R10 promoter (R10-GFP) and a portion of the ε DNaseI HSS cluster. The spleen was stained with lineage specific promoters (Mac-1+, and Gr-1+ are indicative of neutrophils; B220+ is indicative of B cells; CD4, CD8 can be indicative of T cells).

### IKAROS TRANSGENIC ANIMALS AND USES THEREOF

In general, the invention features, a transgenic animal, e.g., a mammal, having an Ikaros transgene.

In preferred embodiments, the mammal is a non-human mammal, e.g., a swine, a monkey, a goat, or a rodent, e.g., a rat, but preferably a mouse.

In preferred embodiments, the Ikaros transgene includes an Ikaros transcriptional control region operably linked to a sequence which is functionally unrelated to the Ikaros gene, or which is less than 60%, 50%, 40%, 30%, or 20% homologous with the Ikaros gene. In a preferred embodiment, the sequence functionally unrelated to Ikaros is a sequence encoding a reporter molecule, a nucleic acid encoding a toxin, or a nucleic acid encoding a gene to be placed under the control of an Ikaros regulatory region. Preferably, the sequence functionally unrelated to Ikaros encodes a reporter molecule which can be detected with relative ease, e.g., a protein, e.g., an enzyme, e.g., an enzyme which produces a colored or luminescent product or emission. In particularly preferred embodiments, the reporter gene can be a beta-galactosidase gene, a luciferase gene, a green fluorescent protein gene, an alkaline phosphatase gene, a horseradish peroxidase gene, or a chloramphenicol acetyl transferase gene. Preferably, the reporter product is capable of providing a signal which indicates the activity of the promoter to which it is linked. Preferred reporters are those which luminesce or fluoresce. Preferred reporters can luminesce or fluoresce, in vivo, without the addition of an exogenous substrate. A particularly suitable reporter is green fluorescent protein. Modified variants of green fluorescent protein, e.g., EGFP, EBFP, EYFP, d2EGFP, ECFP, GFPuv are included within the term green fluorescent protein. These variants of GFP are commercially available by Clontech, Laboratories, Inc. Palo Alto, Calif. Furthermore, GFP and variants thereof, are provided in the following references, all of which are incorporated by reference: Chalfie, M. et al. (1994) Science 263:802-805; Prasher, D. C., et al. (1992) Gene 111:229-233; Inouye, S. & Tsuji, F. I. (1994) FEBS Letters 341:277-280; Wang, S. & Hazelrigg, T. (1994) Nature 369:400-403; Cody, C. W., et al. (1993) Biochemistry 32:1212-1218; Inouye, S. & Tsuji, F. I. (1994) FEBS Letters 351:211-214; Heim, R., et al. (1994) Proc. Natl. Acad. Sci., USA 91:12501-12504; Yang, T. T., et al. (1996) Nucleic Acids Res. 24(22): 4592-4593; Cormack, B. P., et al. (1996) Gene 173:33-38; Crameri, A., et al. (1996) Nature Biotechnol. 12:315-319; Haas, J. et al, (1996) Curr. Biol. 6:315-324; Galbraith, D. W., et al. (1995) Methods Cell Biol. 50:1-12; Living Colors Destabilized EGFP Vectors (April 1998) CLONTECHniques XIII(2):16-17, Living Colors pEBFP Vector (April 1997) CLONTECHniques XII(2):16-17; Heim, R. & Tsien, R. Y. (1996) Curr. Biol. 6:178-182; Ormö, et al. (1996) Science 273:1392-1395; Mitra, R. D. et al. (1996) Gene 173:13-17.

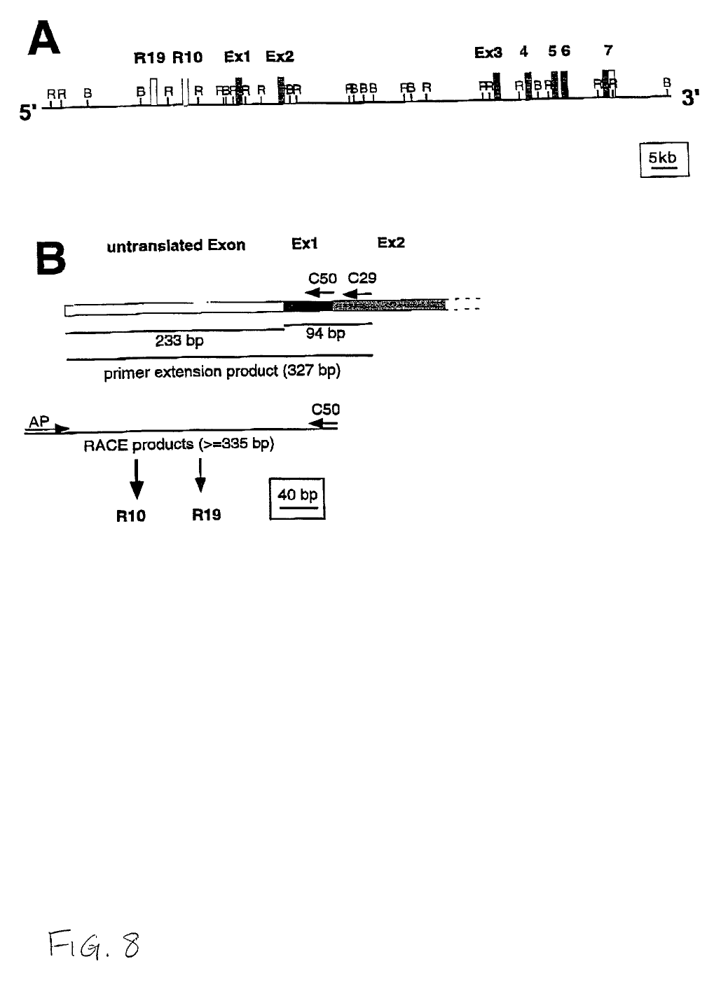

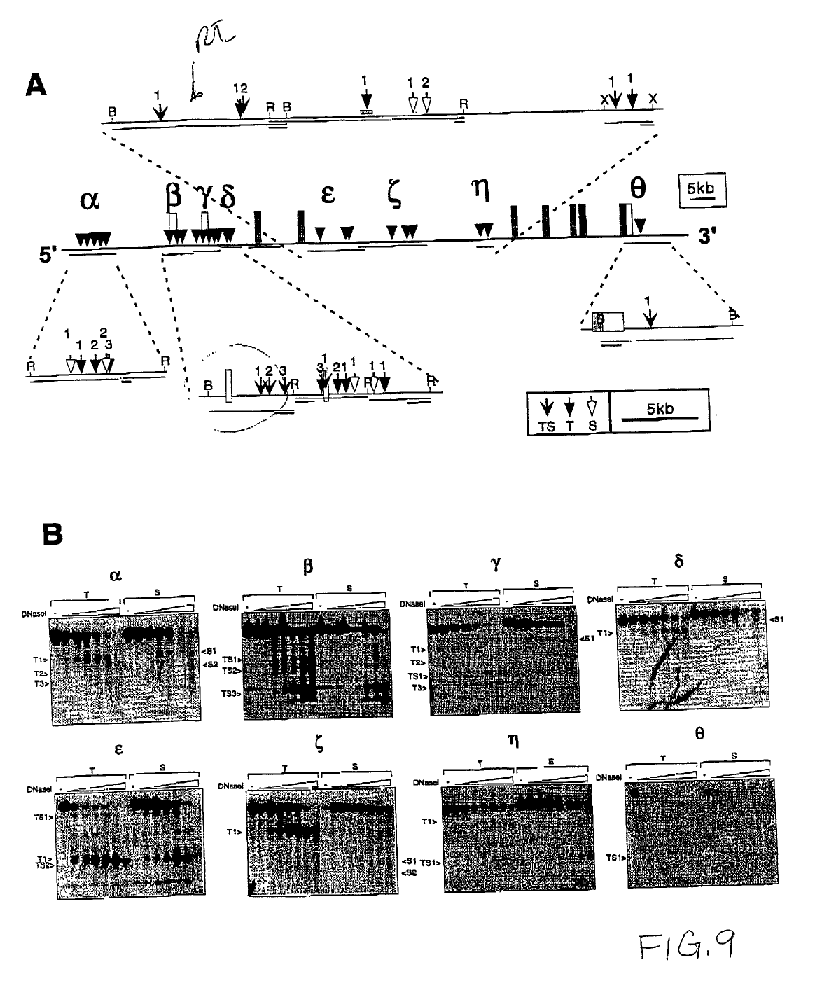

When the Ikaros transgene includes an Ikaros transcriptional control region operably linked to an unrelated sequence, e.g., a sequence encoding a reporter molecule, the transcriptional control region preferably includes one or more Ikaros regulatory elements. Such regulatory elements can include Ikaros promoters, enhancers and/or insulator sequences. The regulatory elements can be 5′ regulatory elements, intronic elements and/or 3′ regulatory elements of Ikaros. In a preferred embodiment, a DNase I HSS cluster of Ikaros includes the regulatory element and all or a portion of the DNase I HSS cluster is included in the transgene. A DNase I HSS cluster, as used herein, refers to a region of the Ikaros gene which includes more than one DNase I HSS. Preferably, the DNase I HSS cluster includes 2, 3, 4 or 5 DNase I HSS within about 0.001, 0.01, 0.1, 0.2, 0.4, 1, 2, 3, 4 kilobases from each other. Examples of such clusters include the α cluster, the β cluster, the γ cluster, the ε cluster, the η cluster and the θ cluster. These clusters in the murine Ikaros gene are shown in FIG. 9A. When the Ikaros transgene includes a portion of a DNase I HSS cluster, the portion can be, e.g., a region including one or more of the DNase I HSS sites in the cluster. For example, a portion of the ε cluster can include one or two of the three DNase I HSS sites of the ε cluster of the murine Ikaros gene.

In a particularly preferred embodiment, the Ikaros transcriptional control region includes: at least a portion of the β cluster containing a promoter, e.g., an R19 promoter, and/or at least a portion of the γ cluster containing a promoter, e.g., an R10promoter. In other embodiments, the Ikaros transcriptional control region can include one or more promoter(s), e.g., a promoter from the β cluster and/or the γ cluster, and one or more Ikaros regulatory element(s), e.g., one or more Ikaros regulatory element from the α cluster, the ε cluster, the η cluster and/or the θ cluster. For example, the Ikaros transcriptional control region can include the γ cluster or a promoter-containing portion thereof and the ε cluster or a portion thereof. In other embodiments, the Ikaros transgene can include all or a promoter-containing portion of the β cluster and/or all or a promoter-containing portion from the γ cluster and: all or a portion of the α cluster; all or a portion of the δ cluster; all or a portion of the ε cluster; all or a portion of the ζ cluster; all or a portion of the η cluster; all or a portion of the θ cluster; combinations of two, three, four, or five of the α cluster, the δ cluster, the ε cluster, the ζ cluster, the η cluster, the θ cluster, or portions thereof; all of the α cluster, the δ cluster, the ε cluster, the ζ cluster, the η cluster and the θ cluster, or portions thereof.

In a preferred embodiment: the transgenic animal further includes a second Ikaros transgene having a mutation. In yet more preferred embodiments, the Ikaros transgene includes a mutation and: the mutation is, or results from, a chromosomal alteration; the mutation is, or results from, any of an alteration resulting from homologous recombination, site-specific recombination, nonhomologous recombination; the mutation is, or results from, any of an inversion, deletion, insertion, translocation, or reciprocal translocation; the mutation is, or results from, any of a deletion of one or more nucleotides from the gene, an addition of one or more nucleotides to the gene, a change of identity of one or more nucleotides of the gene.

In yet other preferred embodiments, the transgenic animal further includes a second Ikaros transgene having a mutation and: the mutation results in mis-expression of the transgene or of another gene in the animal; the mutation results in mis-expression of the transgene and the mis-expression is any of an alteration in the level of a messenger RNA transcript of the transgene, the presence of a non-wild type splicing pattern of a messenger RNA transcript of the transgene, or a non-wild type level of a protein encoded by the transgene; the mutation alters the relative abundance of a first Ikaros isoform with respect to a second Ikaros isoform, as compared, e.g., to a wild type animal or to an animal lacking the transgene; the mutation is in, or alters, the sequence, expression, or splicing of one or more of the following exons: exon 1/2, exon 3, exon 4, exon 5, exon 6, and exon 7; the mutation is in, or alters, the sequence, expression, or splicing of a DNA binding domain of, the Ikaros gene or DNA; the mutation is a deletion of portions of exon 3 and/or exon 4; the mutation is alters the expression, activation, or dimerization of an Ikaros gene product; the mutation is a deletion of a portion of exon 7.

In yet other preferred embodiments, the transgenic animal further includes a second transgene and the second Ikaros transgene encodes: an Ikaros protein which is a competitive inhibitor or an antagonist of a naturally occurring Ikaros protein; an Ikaros gene genetically engineered, e.g., by deletion of an exon, or by using a sequence which results in expression in a preselected tissue, to encode a specific isoform, or a specific subset of Ikaros isoforms, e.g., the transgene is genetically engineered to express one of mIK-1, mIK-2, mIK-3, mIK-4, mIK-5, hIK-l, hIK-2, hIK-3, hIK-4, or hIK-5.

In preferred embodiments, the transgenic animal: is heterozygous for an Ikaros transgene, e.g., a mutated Ikaros transgene; homozygous for an Ikaros transgene, e.g., a mutated Ikaros transgene; includes a first Ikaros transgene, e.g., a transgene which includes an Ikaros transcriptional control region and a sequence unrelated to the Ikaros gene, and a second Ikaros transgene, e.g., a mutated Ikaros transgene; includes an Ikaros transgene, e.g., a transgene which includes an Ikaros transcriptional control region and a sequence unrelated to the Ikaros gene, and a second transgene which is other than an Ikaros transgene, e.g., encoding another polypeptide involved in hematopoiesis, e.g., an Aiolos transgene and/or a Helios transgene, e.g., a mutated Aiolos transgene and/or a mutated Helios transgene.

In another aspect, the invention includes a transgenic mouse having a second transgene and the transgene is a mutated Ikaros transgene, the mutation occurring in, or altering, a domain of the Ikaros gene, e.g., a domain described herein, e.g., the mutation is in, or alters, the sequence of a DNA binding domain of the Ikaros transgene.

In preferred embodiments: the mutation is a deletion of one or more nucleotides from the Ikaros transgene; the mutation is a deletion which is in or which includes a portion of exon 3 and/or exon 4 of the Ikaros transgene.

In another aspect, the invention includes a transgenic mouse having a second transgene and the transgene is a mutated Ikaros transgene in which the mutation alters the expression, activation, or dimerization of an Ikaros gene product.

In preferred embodiments: the mutation is a deletion of one or more nucleotides from the Ikaros transgene; the mutation is a deletion which is in or which includes a portion of exon 7 of the Ikaros transgene.

In another preferred embodiment, the transgenic mouse includes an Ikaros transgene which includes an Ikaros transcriptional control region operably linked to a sequence which is functionally unrelated to the Ikaros gene, as described herein, and a second transgene other than Ikaros. For example, the second transgene can encode another polypeptide involved in hematopoiesis, e.g., an Aiolos and/or Helios transgene. Aiolos is described in PCT Publication Number WO 94/06814, published Mar. 31, 1994, Helios is described in PCT Publication Number WO 99/43288, published Sep. 2, 1999, the contents of which are incorporated herein by reference. In a preferred embodiment, the transgene encoding a polypeptide involved in hematopoiesis other than Ikaros is mutated, e.g., as described herein for mutated Ikaros transgenes. For example, when the second transgene encoding a polypeptide involved in hematopoiesis includes a mutation, the mutation can be, or can result from: a chromosomal alteration; any of an alteration resulting from homologous recombination, site-specific recombination, nonhomologous recombination; any of an inversion, deletion, insertion, translocation, or reciprocal translocation; any of a deletion of one or more nucleotides from the gene, an addition of one or more nucleotides to the gene, a change of identity of one or more nucleotides of the gene. In yet other preferred embodiments, when the second transgene encoding a polypeptide involved in hematopoiesis includes a mutation, the mutation can result in: mis-expression of the transgene or of another gene in the animal; mis-expression of the transgene and the mis-expression is any of an alteration in the level of a messenger RNA transcript of the transgene, the presence of a non-wild type splicing pattern of a messenger RNA transcript of the transgene, or a non-wild type level of a protein encoded by the transgene.

In another aspect, the invention features a method of evaluating a component or lineage of the immune system, e.g., evaluating development of a component or cell lineage of the immune system, e.g., development of a hematpoietic cell of the immune system. The method includes providing a transgenic animal, or cell or tissue therefrom, having an Ikaros transgene which includes an Ikaros transcriptional control region and a sequence encoding a protein functionally unrelated to the Ikaros gene, e.g., a sequence encoding a reporter molecule, and monitoring expression of the protein unrelated to Ikaros, e.g., monitoring expression of the reporter molecule. Preferably, the Ikaros transcriptional control region includes one or more regulatory element(s) of Ikaros which directs expression of the immune component of interest. Types of development which can be evaluated include, e.g., the ontogeny of a component or cell lineage of the immune system, activation of a component or cell lineage of the immune system, the migration of a component or cell lineage of the immune system, regions of action of a component or cell lineage of the immune system and ways in which components or cell lineages of the immune system interact. Examples of immune system components which can be evaluated include hematopoietic cells of the immune system, e.g., hematopoietic stem cells, multipotent progenitors, oligopotent progenitors (e.g., lymphoid or myeloid progenitors), cells committed to the B-cell lineage, cells committed to the T-cell lineage, cells committed to a myeloid cell lineage (e.g., granulocyte monocyte CFU cells), T-lymphocytes, B-lymphocytes, NK cells, and neutrophils.

Development can be evaluated in a living animal, a dead animal, or a cell or tissue taken from a live or dead animal. In a preferred embodiment, the protein unrelated to Ikaros is a reporter molecule, e.g., a colored or fluorescent molecule, and the immune system component is monitored on the live animal. Preferably, the method includes detecting a signal, e.g., a fluorescent signal, on the live animal, e.g., using a confocal microscope in order to monitor expression of the immune system component. Methods of monitoring expression of a reporter molecule in a live animal are described in PCT Publication Number WO 99/30743, published Jun. 24, 1999, the contents of which is incorporated herein by reference.

In a preferred embodiment, the transgenic animal, or cell or tissue therefrom, includes a second transgene. Preferably, the second transgene is a sequence encoding a protein involved in hematopoiesis, e.g., the second transgene encodes an Ikaros polypeptide, an Aiolos polypeptide and/or a Helios polypeptide. The second transgene can encode a mutated transgene which results in altered expression of the transgene, e.g., misexpression of the transgene. Examples of such mutations are described herein.

In one embodiment, the transgenic animal, or cell or tissue therefrom, can include both a first transgene which includes an Ikaros transcriptional control region and a sequence encoding a polypeptide unrelated to Ikaros, e.g., a reporter molecule, and a second transgene which encodes a mutated polypeptide involved in hematopoiesis, e.g., a mutated Ikaros transgene, Aiolos transgene and/or Helios transgene. Preferably, the second transgene is altered such that the polypeptide involved in hematopoiesis is misexpressed, e.g., under-expressed or over-expressed as compared to animals which do not have the mutated second transgene. For example, the mutation in the second transgene can result in decreased expression of the polypeptide involved in hematopoiesis, and the effect of decreased expression, if any, on Ikaros expression can be evaluated by the presence or absence of the reporter expression, e.g., as compared to expression in a transgenic animal that does not have the second mutated transgene.

In another aspect, the invention features a method for evaluating the effect of a treatment on a transgenic cell or animal having an Ikaros transgene. The method includes administering the treatment to a cell or animal having an Ikaros transgene, and evaluating the effect of the treatment on the cell or animal. Preferably, the Ikaros transgene includes an Ikaros transcriptional control region and a sequence functionally unrelated to the Ikaros gene, e.g., a sequence encoding a reporter molecule. The effect can be, e.g., the effect of the treatment on the immune system or a component thereof, the nervous system or a component thereof, or the cell cycle. Immune system effects include e.g., T cell activation, T cell development, B cell development, NK cell development, myeloid cell development, and the ratios CD4⁺/CD8⁺, CD4⁺/CD8⁻ and CD4⁻/CD8⁺.

In preferred embodiments, when using a transgenic animal, the transgenic animal is a mammal, e.g., a non-human mammal, e.g., a nonhuman primate or a swine, a monkey, a goat, or a rodent, e.g., a rat, but preferably a mouse. In other preferred embodiments, the transgenic animal is a fish, e.g., a zebrafish; a nemaotde, e.g., caenorhabditis elegans; an amphibian, e.g., a frog or an axolotl.

In preferred embodiments, when using a transgenic cell, the transgenic cell is a mammalian cell, e.g., a non-human mammalian cell, e.g., a swine, a monkey, a goat, or a rodent, preferably a mouse, cell. In other preferred embodiments, the transgenic cell is from a fish, e.g., a zebrafish; a nemaotde, e.g., caenorhabditis elegans; an amphibian, e.g., a frog or an axolotl.

In other preferred embodiments: the transgenic animal or cell includes a second transgene, e.g., a mutated transgene. The mutated transgene can result, for example, in misexpression of a protein involved in hematopoiesis, e.g., misexpression of Ikaros, Helios and/or Aiolos. In yet more preferred embodiments the second transgene includes a mutation and: the mutation is, or results from, a chromosomal alteration; the mutation is, or results from, any of an alteration resulting from homologous recombination, site-specific recombination, nonhomologous recombination; the mutation is, or results from, any of an inversion, deletion, insertion, translocation, or reciprocal translocation; the mutation is, or results from, any of a deletion of one or more nucleotides from the gene, an addition of one or more nucleotides to the gene, a change of identity of one or more nucleotides of the gene.

In yet other preferred embodiments, the second transgene includes a mutation and: the mutation results in mis-expression of the transgene or of another gene in the animal or cell; the mutation results in mis-expression of the transgene and the mis-expression is any of an alteration in the level of a messenger RNA transcript of the transgene, the presence of a non-wild type splicing pattern of a messenger RNA transcript of the transgene, or a non-wild type level of a protein encoded by the transgene. In a preferred embodiment, the second transgene includes a mutation and: the mutation alters the relative abundance of a first Ikaros isoform with respect to a second Ikaros isoform, as compared, e.g., to a wild type animal or to an animal lacking the transgene; the mutation is in, or alters, the sequence, expression, or splicing of one or more of the following exons: exon 1/2, exon 3, exon 4, exon 5, exon 6, and exon 7; the mutation is in, or alters, the sequence, expression, or splicing of a DNA binding domain of, the Ikaros gene or DNA; the mutation is a deletion of portions of exon 3 and/or exon 4; the mutation is alters the expression, activation, or dimerization of an Ikaros gene product; the mutation is a deletion of a portion of exon 7.

In yet other preferred embodiments, the second transgene encodes: an Ikaros protein which is a competitive inhibitor or an antagonist of a naturally occurring Ikaros protein; an Ikaros gene genetically engineered, e.g., by deletion of an exon, or by using a sequence which results in expression in a preselected tissue, to encode a specific isoform, or a specific subset of Ikaros isoforms, e.g., the transgene is genetically engineered to express one of mIK-1, mIK-2, mIK-3, mIK-4, mIK-5, hIK-1, hIK-2, hIK-3, hIK-4, or hIK-5.

In preferred embodiments, the transgenic animal or cell: is heterozygous for an Ikaros transgene, e.g., a mutated Ikaros transgene; homozygous for an Ikaros transgene, e.g., a mutated Ikaros transgene; includes a first Ikaros transgene, e.g., a transgene which includes an Ikaros transcriptional control region and a sequence unrelated to the Ikaros gene, and a second Ikaros transgene, e.g., a mutated Ikaros transgene; includes an Ikaros transgene, e.g., a transgene which includes an Ikaros transcriptional control region and a sequence unrelated to the Ikaros gene, and a second transgene which is other than an Ikaros transgene, e.g., an Aiolos transgene and/or a Helios transgene, e.g., a mutated Aiolos transgene and/or a mutated Helios transgene.

In preferred embodiments, the evaluating step includes determining the effect of the treatment on a parameter related to the immune system. The parameter related to the immune system can, e.g., be any of: the presence, function, or morphology of T cells or their progenitors: the presence, function, or morphology of B cells or their progenitors; the presence, function, or morphology of natural killer cells or their progenitors; the presence function, or morphology of myeloid cells, e.g., neutrophils, or their progenitors; resistance to infection; life span; body weight; the presence, function, or morphology of tissues or organs of the immune system; the expression of the Ikaros transgene; the ability of a component of the immune system to respond to a stimulus (e.g., a diffusible substance, e.g., cytokines, other cells of the immune system, or antigens); the ability to exhibit immunological tolerance to an alloantigen or a xenoantigen.

In preferred embodiments, the evaluating step includes evaluating the expression of the sequence unrelated to the Ikaros gene, e.g., expression of the sequence encoding a reporter molecule.

In preferred embodiments, the treatment can include: the administration of a drug, chemical, or other substance; the administration of ionizing radiation; the administration of an antibody, e.g., an antibody directed against a molecule or cell of the immune system, e.g., an antibody directed against a T cell, B cell, NK cell, dendritic cell, or thymic cell, an antibody directed against a precursor of a T cell, B cell, NK cell, dendritic cell, or thymic cell, an antibody directed against a cell surface marker of a T cell, B cell, NK cell, dendritic cell, or thymic cell; introduction of a component of the immune system derived from an animal of the same species as the transgenic animal; the introduction of a component of the immune system derived from an animal of a different species from the transgenic animal; the introduction of an immune system component derived from an animal or cell other than the transgenic animal or cell; the introduction of an immune system component which is endogenous, (i.e., it is present in the transgenic animal or cell and does not have to be introduced into the transgenic animal or cell) to the transgenic animal or cell; the introduction of an immune system component derived from an animal or cell of the same species as the transgenic animal or cell; the introduction of an immune system component derived from an animal or cell (of the same species as the transgenic animal) which does not include the transgene; the introduction of an immune system component derived from an immunologically competent animal, or from a cell derived from an immunologically competent animal, of the same species as the transgenic animal or cell; the introduction of an immune system component derived from an animal or cell of a different species from the transgenic animal or cell; the introduction of an immune system component derived from an immunologically competent animal, or from a cell derived from an immunologically competent animal, of a different species than the transgenic animal or cell; administration of a substance or other treatment which suppresses the immune system; administration of a substance or other treatment which activates or boosts the function of the immune system; introduction of a nucleic acid, e.g., a nucleic acid which encodes or expresses a component of the immune system; or the introduction of a protein, e.g., a protein which is a component of the immune system.

In another aspect, the invention features a method for evaluating the effect of a treatment on an immune system component. The method includes: (1) supplying a transgenic cell or animal having an Ikaros transgene; (2) supplying the immune system component; (3) administering the treatment; and (4) evaluating the effect of the treatment on the immune system component.

In preferred embodiments using a transgenic animal the transgenic animal is a mammal, e.g., a non-human mammal, e.g., a nonhuman primate or a swine, a monkey, a goat, or a rodent, e.g., a rat, but preferably a mouse. In other preferred embodiments, the transgenic animal is a fish, e.g., a zebrafish; a nemaotde, e.g., caenorhabditis elegans; an amphibian, e.g., a frog or an axolotl.

In preferred embodiments using a transgenic cell the transgenic cell is a mammalian cell, e.g., a non-human mammalian cell, e.g., a swine, a monkey, a goat, or a rodent, preferably a mouse, cell. In other preferred embodiments, the transgenic cell is from a fish, e.g., a zebrafish; a nemaotde, e.g., caenorhabditis elegans; an amphibian, e.g., a frog or an axolotl.

In other preferred embodiments: the Ikaros transgene includes a mutation. In yet more preferred embodiments the Ikaros transgene includes a mutation and: the mutation is, or results from, a chromosomal alteration; the mutation is, or results from, any of an alteration resulting from homologous recombination, site-specific recombination, nonhomologous recombination; the mutation is, or results from, any of an inversion, deletion, insertion, translocation, or reciprocal translocation; the mutation is, or results from, any of a deletion of one or more nucleotides from the gene, an addition of one or more nucleotides to the gene, a change of identity of one or more nucleotides of the gene.

In yet other preferred embodiments the Ikaros transgene includes a mutation and: the mutation results in mis-expression of the transgene or of another gene in the animal; the mutation results in mis-expression of the transgene and the mis-expression is any of an alteration in the level of a messenger RNA transcript of the transgene, the presence of a non-wild type splicing pattern of a messenger RNA transcript of the transgene, or a non-wild type level of a protein encoded by the transgene; the mutation alters the relative abundance of a first Ikaros isoform with respect to a second Ikaros isoform, as compared, e.g., to a wild type animal or to an animal lacking the transgene; the mutation is in, or alters, the sequence, expression, or splicing of one or more of the following exons: exon 1/2, exon 3, exon 4, exon 5, exon 6, and exon 7; the mutation is in, or alters, the sequence, expression, or splicing of a DNA binding domain of, the Ikaros gene or DNA; the mutation is a deletion of portions of exon 3 and/or exon 4; the mutation is alters the expression, activation, or dimerization of an Ikaros gene product; the mutation is a deletion of a portion of exon 7.

In yet other preferred embodiments the Ikaros transgene includes an Ikaros transcriptional control region operably linked to a sequence which is functionally unrelated to the Ikaros gene, or which is less than 50% homologous with the Ikaros gene, e.g., a nucleic acid encoding a reporter molecule, a nucleic acid encoding a toxin, or a nucleic acid encoding a gene to be placed under the control of an Ikaros regulatory region.

In yet other preferred embodiments the Ikaros transgene encodes: an Ikaros protein which is a competitive inhibitor or an antagonist of a naturally occurring Ikaros protein; an Ikaros gene genetically engineered, e.g., by deletion of an exon, or by using a sequence which results in expression in a preselected tissue, to encode a specific isoform, or a specific subset of Ikaros isoforms, e.g., the transgene is genetically engineered to express one of mIK-1, mIK-2, mIK-3, mIK-4, mIK-5, hIK-1, hIK-2, hIK-3, hIK-4, or hIK-5.

In preferred embodiments the transgenic animal or cell: is heterozygous for an Ikaros transgene; homozygous for an Ikaros transgene; includes a first Ikaros transgene and a second Ikaros transgene; includes an Ikaros transgene and a second transgene which is other than an Ikaros transgene.

In preferred embodiments: the immune system component is taken from an animal or cell other than the transgenic animal or cell and is introduced into the transgenic cell or animal; the component is endogenous, to the transgenic animal or cell; the immune system component is taken from an animal or cell of the same species as the transgenic animal or cell and is introduced into the transgenic cell or animal (i.e., it is present in the transgenic animal or cell and does not have to be introduced into the transgenic animal or cell); the immune system component is taken from an animal or cell (of the same species as the transgenic animal) which does not include the transgene and is introduced into the transgenic cell or animal; the immune system component is taken from an immunologically competent animal, or from a cell derived from an immunologically competent animal, of the same species as the transgenic animal or cell and is introduced into the transgenic cell or animal; the immune system component is taken from an animal or cell of a different species from the transgenic animal or cell and is introduced into the transgenic cell or animal; the immune system component is taken from an immunologically competent animal, or from a cell derived from an immunologically competent animal, of a different species than the transgenic animal or cell and is introduced into the transgenic cell or animal.

In preferred embodiments the immune system component is any of an antigen, a T cell, a T cell progenitor, a totipotent hematopoietic stem cell, a pluripotent hematopoietic stem cell, a B cell, a B cell progenitor, a natural killer cell, a natural killer cell progenitor, bone marrow tissue, spleen tissue, or thymic tissue.

In other preferred embodiments the immune system component is: a nucleic acid which encodes an immune system component, e.g., a cell surface marker, a receptor, or a cytokine; a protein, e.g., a cell surface marker, a receptor, or a cytokine.

In preferred embodiments, the evaluating step includes determining the effect of the treatment on a parameter related to the immune system. The parameter related to the immune system can, e.g., be any of: the presence, function, or morphology of T cells or their progenitors: the presence, function, or morphology of B cells or their progenitors; the presence, function, or morphology of natural killer cells or their progenitors; resistance to infection; life span; body weight; the presence, function, or morphology of tissues or organs of the immune system; the expression of the Ikaros transgene; the ability of a component of the immune system to respond to a stimulus (e.g., a diffusible substance, e.g., cytokines, other cells of the immune system, or antigens); the ability to exhibit immunological tolerance to an alloantigen or a xenoantigen.

In preferred embodiments the evaluating step includes evaluating the expression of a gene or transgene, e.g., a gene which encodes a component of the immune system, e.g., a cell surface marker, a receptor, or a cytokine; a gene which regulates the expression of a component of the immune system, a gene which modulates the ability of the immune system to function, the Ikaros gene or an Ikaros transgene.

In preferred embodiments the evaluating step includes evaluating the growth rate of a transgenic cell.

In preferred embodiments the treatment can include: the administration of a drug, chemical, or other substance; the administration of ionizing radiation; the administration of an antibody, e.g., an antibody directed against a molecule or cell of the immune system, e.g., an antibody directed against a T cell, B cell, NK cell, dendritic cell, or thymic cell, an antibody directed against a precursor of a T cell, B cell, NK cell, dendritic cell, or thymic cell, an antibody directed against a cell surface marker of a T cell, B cell, NK cell, dendritic cell, or thymic cell; introduction of a component of the immune system derived from an animal or cell of the same species as the transgenic animal or cell; the introduction of a component of the immune system derived from an animal or cell of a different species from the transgenic animal or cell; the introduction of an immune system component derived from an animal or cell other than the transgenic animal or cell; the introduction of an immune system component which is endogenous, (i.e., it is present in the transgenic animal or cell and does not have to be introduced into the transgenic animal or cell) to the transgenic animal or cell; the introduction of an immune system component derived from an animal or cell of the same species as the transgenic animal or cell ; the introduction of an immune system component derived from an animal or cell (of the same species as the transgenic animal) which does not include the transgene; the introduction of an immune system component derived from an immunologically competent animal, or from a cell derived from an immunologically competent animal, of the same species as the transgenic animal or cell; the introduction of an immune system component derived from an animal or cell of a different species from the transgenic animal or cell; the introduction of an immune system component derived from an immunologically competent animal, or from a cell derived from an immunologically competent animal, of a different species than the transgenic animal or cell; administration of a substance or other treatment which suppresses the immune system; or administration of a substance or other treatment which activates or boosts the function of the immune system; introduction of a nucleic acid, e.g., a nucleic acid which encodes or expresses a component of the immune system; the introduction of a protein, e.g., a protein which is a component of the immune system.

In yet another aspect, the invention features a method for evaluating the interaction of a first immune system component with a second immune system component. The method includes: (1) supplying a transgenic cell or animal, e.g., a mammal, having an Ikaros transgene; (2) introducing the first and second immune system component into the transgenic cell or mammal; and (3) evaluating an interaction between the first and second immune system components.

In preferred embodiments, with respect to either the first and/or the second immune system component: the immune system component is taken from an animal or cell other than the transgenic cell or animal and is introduced into the transgenic cell or animal; the component is endogenous, (i.e., it is present in the transgenic animal or cell and does not have to be introduced into the transgenic animal or cell) to the transgenic animal or cell; the immune system component is taken from an animal or cell of the same species as the transgenic animal or cell and is introduced into the transgenic cell or animal; the immune system component is taken from an animal or cell (of the same species as the transgenic animal) which does not include the transgene and is introduced into the transgenic cell or animal; the immune system component is taken from an immunologically competent animal, or from a cell derived from an immunologically competent animal, of the same species as the transgenic animal or cell and is introduced into the transgenic cell or animal; the immune system component is taken from an animal or cell of a different species from the transgenic animal or cell and is introduced into the transgenic cell or animal; the immune system component is taken from an immunologically competent animal, or from a cell derived from an immunologically competent animal, of a different species than the transgenic animal or cell and is introduced into the transgenic cell or animal.

In preferred embodiments the immune system component is any of an antigen, a T cell, a T cell progenitor, a totipotent hematopoietic stem cell, a pluripotent hematopoietic stem cell, a B cell, a B cell progenitor, a natural killer cell, a natural killer cell progenitor, bone marrow tissue, spleen tissue, thymic tissue, or other lymphoid tissue and its stroma, e.g., encapsulated lymphoid tissue, e.g., lymph nodes, or unencapsulated lymphoid tissue, e.g., Peyer's patches in the ileum, lymphoid nodules found in the mucosa of the alimentary, respiratory, urinary, and reproductive tracts.

In other preferred embodiments the immune system component is: a nucleic acid which encodes an immune system component, e.g., a cell surface marker, a receptor, or a cytokine; a protein, e.g., a cell surface marker, a receptor, or a cytokine.

In preferred embodiments, the first component is the same as the second component; the first component is different from the second component; the first and the second components are from the same species as the transgenic mammal; the first and the second components are from species different from the species of the transgenic mammal; the first and second components are from different species.

In preferred embodiments, when using a transgenic animal, the transgenic animal is a mammal, e.g., a non-human mammal, e.g., a nonhuman primate or a swine, a monkey, a goat, or a rodent, e.g., a rat, but preferably a mouse. In other preferred embodiments, the transgenic animal is a fish, e.g., a zebrafish; a nemaotde, e.g., caenorhabditis elegans; an amphibian, e.g., a frog or an axolotl.

In preferred embodiments, when using a transgenic cell, the transgenic cell is a mammalian cell, e.g., a non-human mammalian cell, e.g., a swine, a monkey, a goat, or a rodent, preferably a mouse, cell. In other preferred embodiments, the transgenic cell is from a fish, e.g., a zebrafish; a nemaotde, e.g., caenorhabditis elegans; an amphibian, e.g., a frog or an axolotl.

In other preferred embodiments: the Ikaros transgene includes a mutation. In yet more preferred embodiments, the Ikaros transgene includes a mutation and: the mutation is, or results from, a chromosomal alteration; the mutation is, or results from, any of an alteration resulting from homologous recombination, site-specific recombination, nonhomologous recombination; the mutation is, or results from, any of an inversion, deletion, insertion, translocation, or reciprocal translocation; the mutation is, or results from, any of a deletion of one or more nucleotides from the gene, an addition of one or more nucleotides to the gene, a change of identity of one or more nucleotides of the gene.

In yet other preferred embodiments, the Ikaros transgene includes a mutation and: the mutation results in mis-expression of the transgene or of another gene in the animal; the mutation results in mis-expression of the transgene and the mis-expression is any of an alteration in the level of a messenger RNA transcript of the transgene, the presence of a non-wild type splicing pattern of a messenger RNA transcript of the transgene, or a non-wild type level of a protein encoded by the transgene; the mutation alters the relative abundance of a first Ikaros isoform with respect to a second Ikaros isoform, as compared, e.g., to a wild type animal or to an animal lacking the transgene; the mutation is in, or alters, the sequence, expression, or splicing of one or more of the following exons: exon 1/2, exon 3, exon 4, exon 5, exon 6, and exon 7; the mutation is in, or alters, the sequence, expression, or splicing of a DNA binding domain of, the Ikaros gene or DNA; the mutation is a deletion of portions of exon 3 and/or exon 4; the mutation is alters the expression, activation, or dimerization of an Ikaros gene product; the mutation is a deletion of a portion of exon 7.

In yet other preferred embodiments, the Ikaros transgene includes an Ikaros transcriptional control region operably linked to a sequence which is functionally unrelated to the Ikaros gene, or which is less than 50 % homologous with the Ikaros gene, e.g., a nucleic acid encoding a reporter molecule, a nucleic acid encoding a toxin, or a nucleic acid encoding a gene to be placed under the control of an Ikaros regulatory region.

In yet other preferred embodiments, the Ikaros transgene encodes: an Ikaros protein which is a competitive inhibitor or an antagonist of a naturally occurring Ikaros protein; an Ikaros gene genetically engineered, e.g., by deletion of an exon, or by using a sequence which results in expression in a preselected tissue, to encode a specific isoform, or a specific subset of Ikaros isoforms, e.g., the transgene is genetically engineered to express one of mIK-1, mIK-2, mIK-3, mIK-4, mIK-5, hIK-1, hIK-2, hIK-3, hIK-4, or hIK-5.

In preferred embodiments, the transgenic animal or cell: is heterozygous for an Ikaros transgene; homozygous for an Ikaros transgene; includes a first Ikaros transgene and a second Ikaros transgene; includes an Ikaros transgene and a second transgene which is other than an Ikaros transgene.

In preferred embodiments, the evaluating step includes determining the effect of the treatment on a parameter related to the immune system. The parameter related to the immune system can, e.g., be any of: the presence, function, or morphology of T cells or their progenitors: the presence, function, or morphology of B cells or their progenitors; the presence, function, or morphology of natural killer cells or their progenitors; resistance to infection; life span; body weight; the presence, function, or morphology of tissues or organs of the immune system; the expression of the Ikaros transgene; the ability of a component of the immune system to respond to a stimulus (e.g., a diffusible substance, e.g., cytokines, other cells of the immune system, or antigens); the ability to exhibit immunological tolerance to an alloantigen or a xenoantigen.

In preferred embodiments, the evaluating step includes evaluating the expression of a gene or transgene, e.g., a gene which encodes a component of the immune system, e.g., a cell surface marker, a receptor, or a cytokine; a gene which regulates the expression of a component of the immune system, a gene which modulates the ability of the immune system to function, the Ikaros gene or an Ikaros transgene.

In preferred embodiments, the evaluating step includes evaluating the growth rate of a transgenic cell.

In another aspect, the invention features a method for evaluating the effect of a treatment on an immune system disorder including: administering the treatment to a cell or animal having an Ikaros transgene, and evaluating the effect of the treatment on the cell or animal.

In preferred embodiments, the disorder is: a neoplastic disorder; a lymphoma; a T cell related lymphoma.

In preferred embodiments, when using a transgenic animal, the transgenic animal is a mammal, e.g., a non-human mammal, e.g., a swine, a monkey, a goat, or a rodent, e.g., a rat, but preferably a mouse.

In preferred embodiments, when using a transgenic cell, the transgenic cell is a mammalian cell, e.g., a non-human mammalian cell, e.g., a swine, a monkey, a goat, or a rodent, preferably a mouse, cell.

In other preferred embodiments: the Ikaros transgene includes a mutation. In yet more preferred embodiments, the Ikaros transgene includes a mutation and: the mutation is, or results from, a chromosomal alteration; the mutation is, or results from, any of an alteration resulting from homologous recombination, site-specific recombination, nonhomologous recombination; the mutation is, or results from, any of an inversion, deletion, insertion, translocation, or reciprocal translocation; the mutation is, or results from, any of a deletion of one or more nucleotides from the gene, an addition of one or more nucleotides to the gene, a change of identity of one or more nucleotides of the gene.

In yet other preferred embodiments, the Ikaros transgene includes a mutation and: the mutation results in mis-expression of the transgene or of another gene in the animal; the mutation results in mis-expression of the transgene and the mis-expression is any of an alteration in the level of a messenger RNA transcript of the transgene, the presence of a non-wild type splicing pattern of a messenger RNA transcript of the transgene, or a non-wild type level of a protein encoded by the transgene; the mutation alters the relative abundance of a first Ikaros isoform with respect to a second Ikaros isoform, as compared, e.g., to a wild type animal or to an animal lacking the transgene; the mutation is in, or alters, the sequence, expression, or splicing of one or more of the following exons: exon 1/2, exon 3, exon 4, exon 5, exon 6, and exon 7; the mutation is in, or alters, the sequence, expression, or splicing of a DNA binding domain of, the Ikaros gene or DNA; the mutation is a deletion of portions of exon 3 and/or exon 4; the mutation is alters the expression, activation, or dimerization of an Ikaros gene product; the mutation is a deletion of a portion of exon 7.

In yet other preferred embodiments, the Ikaros transgene includes an Ikaros transcriptional control region operably linked to a sequence which is functionally unrelated to the Ikaros gene, or which is less than 50% homologous with the Ikaros gene, e.g., a nucleic acid encoding a reporter molecule, or a nucleic acid encoding a gene to be placed under the control of an Ikaros regulatory region.

In yet other preferred embodiments, the Ikaros transgene encodes: an Ikaros protein which is a competitive inhibitor or an antagonist of a naturally occurring Ikaros protein; an Ikaros gene genetically engineered, e.g., by deletion of an exon, or by using a sequence which results in expression in a preselected tissue, to encode a specific isoform, or a specific subset of Ikaros isoforms, e.g., the transgene is genetically engineered to express one of mIK-1, mIK-2, mIK-3, mIK-4, mIK-5, hIK-1, hIK-2, hIK-3, hIK-4, or hIK-5.

In preferred embodiments, the transgenic animal or cell: is heterozygous for an Ikaros transgene; homozygous for an Ikaros transgene; includes a first Ikaros transgene and a second Ikaros transgene; includes an Ikaros transgene and a second transgene which is other than an Ikaros transgene.

In preferred embodiments, the evaluating step includes determining the effect of the treatment on a parameter related to the immune system. The parameter related to the immune system can, e.g., be any of: the presence, function, or morphology of T cells or their progenitors: the presence, function, or morphology of B cells or their progenitors; the presence, function, or morphology of natural killer cells or their progenitors; resistance to infection; life span; body weight; the presence, function, or morphology of tissues or organs of the immune system; the expression of the Ikaros transgene; the ability of a component of the immune system to respond to a stimulus (e.g., a diffusible substance, e.g., cytokines, other cells of the immune system, or antigens); the ability to exhibit immunological tolerance to an alloantigen or a xenoantigen.

In preferred embodiments, the evaluating step includes evaluating the expression of a gene or transgene, e.g., a gene which encodes a component of the immune system, e.g., a cell surface marker, a receptor, or a cytokine; a gene which regulates the expression of a component of the immune system, a gene which modulates the ability of the immune system to function, the Ikaros gene or an Ikaros transgene.

In preferred embodiments, the evaluating step includes evaluating the growth rate of a transgenic cell.

In preferred embodiments, the treatment can include: the administration of a drug, chemical, or other substance; the administration of ionizing radiation; the administration of an antibody, e.g., an antibody directed against a molecule or cell of the immune system, e.g., an antibody directed against a T cell, B cell, NK cell, dendritic cell, or thymic cell, an antibody directed against a precursor of a T cell, B cell, NK cell, dendritic cell, or thymic cell, an antibody directed against a cell surface marker of a T cell, B cell, NK cell, dendritic cell, or thymic cell; introduction of a component of the immune system derived from an animal of the same species as the transgenic animal; the introduction of a component of the immune system derived from an animal of a different species from the transgenic animal; the introduction of an immune system component derived from an animal or cell other than the transgenic animal or cell; the introduction of an immune system component which is endogenous, (i.e., it is present in the transgenic animal or cell and does not have to be introduced into the transgenic animal or cell) to the transgenic animal or cell; the introduction of an immune system component derived from an animal or cell of the same species as the transgenic animal or cell ; the introduction of an immune system component derived from an animal or cell (of the same species as the transgenic animal) which does not include the transgene; the introduction of an immune system component derived from an immunologically competent animal, or from a cell derived from an immunologically competent animal, of the same species as the transgenic animal or cell; the introduction of an immune system component derived from an animal or cell of a different species from the transgenic animal or cell; the introduction of an immune system component derived from an immunologically competent animal, or from a cell derived from an immunologically competent animal, of a different species than the transgenic animal or cell; administration of a substance or other treatment which suppresses the immune system; or administration of a substance or other treatment which activates or boosts the function of the immune system; introduction of a nucleic acid, e.g., a nucleic acid which encodes or expresses a component of the immune system; the introduction of a protein, e.g., a protein which is a component of the immune system.

In another aspect, the invention features a method for evaluating the effect of a treatment on the nervous system including administering the treatment to a transgenic cell or an animal having an Ikaros transgene, and evaluating the effect of the treatment on the cell or the animal.

In preferred embodiments, when using a transgenic animal, the transgenic animal is a mammal, e.g., a non-human mammal, e.g., a swine, a monkey, a goat, or a rodent, e.g., a rat, but preferably a mouse.

In preferred embodiments, when using a transgenic cell, the transgenic cell is a mammalian cell, e.g., a non-human mammalian cell, e.g., a swine, a monkey, a goat, or a rodent, preferably a mouse, cell.

In other preferred embodiments: the Ikaros transgene includes a mutation. In yet more preferred embodiments, the Ikaros transgene includes a mutation and: the mutation is, or results from, a chromosomal alteration; the mutation is, or results from, any of an alteration resulting from homologous recombination, site-specific recombination, nonhomologous recombination; the mutation is, or results from, any of an inversion, deletion, insertion, translocation, or reciprocal translocation; the mutation is, or results from, any of a deletion of one or more nucleotides from the gene, an addition of one or more nucleotides to the gene, a change of identity of one or more nucleotides of the gene.

In yet other preferred embodiments, the Ikaros transgene includes a mutation and: the mutation results in mis-expression of the transgene or of another gene in the animal; the mutation results in mis-expression of the transgene and the mis-expression is any of an alteration in the level of a messenger RNA transcript of the transgene, the presence of a non-wild type splicing pattern of a messenger RNA transcript of the transgene, or a non-wild type level of a protein encoded by the transgene; the mutation alters the relative abundance of a first Ikaros isoform with respect to a second Ikaros isoform, as compared, e.g., to a wild type animal or to an animal lacking the transgene; the mutation is in, or alters, the sequence, expression, or splicing of one or more of the following exons: exon 1/2, exon 3, exon 4, exon 5, exon 6, and exon 7; the mutation is in, or alters, the sequence, expression, or splicing of a DNA binding domain of, the Ikaros gene or DNA; the mutation is a deletion of portions of exon 3 and/or exon 4; the mutation is alters the expression, activation, or dimerization of an Ikaros gene product; the mutation is a deletion of a portion of exon 7.

In yet other preferred embodiments, the Ikaros transgene includes an Ikaros transcriptional control region operably linked to a sequence which is functionally unrelated to the Ikaros gene, or which is less than 50% homologous with the Ikaros gene, e.g., a nucleic acid encoding a reporter molecule, a nucleic acid encoding a toxin, or a nucleic acid encoding a gene to be placed under the control of an Ikaros regulatory region.

In yet other preferred embodiments, the Ikaros transgene encodes: an Ikaros protein which is a competitive inhibitor or an antagonist of a naturally occurring Ikaros protein; an Ikaros gene genetically engineered, e.g., by deletion of an exon, or by using a sequence which results in expression in a preselected tissue, to encode a specific isoform, or a specific subset of Ikaros isoforms, e.g., the transgene is genetically engineered to express one of mIK-1, mIK-2, mIK-3, mIK-4, mIK-5, hIK-l, hIK-2, hIK-3, hIK-4, or hIK-5.

In preferred embodiments, the transgenic animal or cell: is heterozygous for an Ikaros transgene; homozygous for an Ikaros transgene; includes a first Ikaros transgene and a second Ikaros transgene; includes an Ikaros transgene and a second transgene which is other than an Ikaros transgene.

In preferred embodiments, the evaluating step includes determining the effect of the treatment on a parameter related to the nervous system. The parameter related to the nervous system can, e.g., be any of: the presence, function, or morphology of cells (or their progenitors) of a nervous tissue, e.g., neurons, glial cells, brain cells, or cells of the basal ganglia, e.g., cells of the corpus striatum, cells of the substantia nigra; resistance to infection; life span; body weight; the presence, function, or morphology of tissues or organs of the nervous system; the expression of a gene, e.g., the Ikaros transgene.

In preferred embodiments, the evaluating step includes evaluating the expression of a gene or transgene, e.g., a gene which encodes a component of the nervous system, e.g., a cell surface marker, or a receptor, the Ikaros gene, or an Ikaros transgene.

In preferred embodiments, the evaluating step includes evaluating the growth rate of a transgenic cell.

In preferred embodiments, the treatment can include: the administration of a drug, chemical, or other substance; the administration of ionizing radiation; the administration of an antibody, e.g., an antibody directed against a molecule or cell of the nervous system; administration of a substance or other treatment which suppresses the immune system; or administration of a substance or other treatment which activates or boosts the function of the immune system; introduction of a nucleic acid, e.g., a nucleic acid which encodes or expresses a component of the nervous system; the introduction of a protein, e.g., a protein which is a component of the immune system.

In another aspect, the invention features, a method for evaluating the effect of a treatment on a disorder of the nervous system including administering the treatment to a cell or animal having an Ikaros transgene, and evaluating the effect of the treatment on the cell or animal.

In preferred embodiments, the disorder is: related to the presence, function, or morphology of cells (or their progenitors) of a nervous tissue, e.g., neurons, glial cells, brain cells, or cells of the basal ganglia, e.g., cells of the corpus striatum, cells of the substantia nigra; trauma; Alzheimer's disease; Parkinson's disease; or Huntington's disease.

In preferred embodiments, when using a transgenic animal, the transgenic animal is a mammal, e.g., a non-human mammal, e.g., a nonhuman primate or a swine, a monkey, a goat, or a rodent, e.g., a rat, but preferably a mouse. In other preferred embodiments, the transgenic animal is a fish, e.g., a zebrafish; a nemaotde, e.g., caenorhabditis elegans; an amphibian, e.g., a frog or an axolotl.

In preferred embodiments, when using a transgenic cell, the transgenic cell is a mammalian cell, e.g., a non-human mammalian cell, e.g., a swine, a monkey, a goat, or a rodent, preferably a mouse, cell. In other preferred embodiments, the transgenic cell is from a fish, e.g., a zebrafish; a nemaotde, e.g., caenorhabditis elegans; an amphibian, e.g., a frog or an axolotl.

In other preferred embodiments: the Ikaros transgene includes a mutation. In yet more preferred embodiments, the Ikaros transgene includes a mutation and: the mutation is, or results from, a chromosomal alteration; the mutation is, or results from, any of an alteration resulting from homologous recombination, site-specific recombination, nonhomologous recombination; the mutation is, or results from, any of an inversion, deletion, insertion, translocation, or reciprocal translocation; the mutation is, or results from, any of a deletion of one or more nucleotides from the gene, an addition of one or more nucleotides to the gene, a change of identity of one or more nucleotides of the gene.

In yet other preferred embodiments, the Ikaros transgene includes a mutation and: the mutation results in mis-expression of the transgene or of another gene in the animal; the mutation results in mis-expression of the transgene and the mis-expression is any of an alteration in the level of a messenger RNA transcript of the transgene, the presence of a non-wild type splicing pattern of a messenger RNA transcript of the transgene, or a non-wild type level of a protein encoded by the transgene; the mutation alters the relative abundance of a first Ikaros isoform with respect to a second Ikaros isoform, as compared, e.g., to a wild type animal or to an animal lacking the transgene; the mutation is in, or alters, the sequence, expression, or splicing of one or more of the following exons: exon 1/2, exon 3, exon 4, exon 5, exon 6, and exon 7; the mutation is in, or alters, the sequence, expression, or splicing of a DNA binding domain of, the Ikaros gene or DNA; the mutation is a deletion of portions of exon 3 and/or exon 4; the mutation is alters the expression, activation, or dimerization of an Ikaros gene product; the mutation is a deletion of a portion of exon 7.

In yet other preferred embodiments, the Ikaros transgene includes an Ikaros transcriptional control region operably linked to a sequence which is functionally unrelated to the Ikaros gene, or which is less than 50% homologous with the Ikaros gene, e.g., a nucleic acid encoding a reporter molecule, a nucleic acid encoding a toxin, or a nucleic acid encoding a gene to be placed under the control of an Ikaros regulatory region.

In yet other preferred embodiments, the Ikaros transgene encodes: an Ikaros protein which is a competitive inhibitor or an antagonist of a naturally occurring Ikaros protein; an Ikaros gene genetically engineered, e.g., by deletion of an exon, or by using a sequence which results in expression in a preselected tissue, to encode a specific isoform, or a specific subset of Ikaros isoforms, e.g., the transgene is genetically engineered to express one of mIK-1, mIK-2, mIK-3, mIK-4, mIK-5, hIK-1, hIK-2, hIK-3, hIK-4, or hIK-5.

In preferred embodiments the transgenic animal or cell: is heterozygous for an Ikaros transgene; homozygous for an Ikaros transgene; includes a first Ikaros transgene and a second Ikaros transgene; includes an Ikaros transgene and a second transgene which is other than an Ikaros transgene.

In preferred embodiments, the evaluating step includes determining the effect of the treatment on a parameter related to the nervous system. The parameter related to the nervous system can, e.g., be any of: the presence, function, or morphology of cells (or their progenitors) of a nervous tissue, e.g., neurons, glial cells, brain cells, or cells of the basal ganglia, e.g., cells of the corpus striatum, cells of the substantia nigra; resistance to infection; life span; body weight; the presence, function, or morphology of tissues or organs of the nervous system; the expression of a gene, e.g., the Ikaros transgene.

In preferred embodiments, the evaluating step includes evaluating the expression of a gene or transgene, e.g., a gene which encodes a component of the nervous system, e.g., a cell surface marker, or a receptor, the Ikaros gene, or an Ikaros transgene.

In preferred embodiments, the evaluating step includes evaluating the growth rate of a transgenic cell.

In preferred embodiments, the treatment can include: the administration of a drug, chemical, or other substance; the administration of ionizing radiation; the administration of an antibody, e.g., an antibody directed against a molecule or cell of the nervous system; administration of a substance or other treatment which suppresses the immune system; or administration of a substance or other treatment which activates or boosts the function of the immune system; introduction of a nucleic acid, e.g., a nucleic acid which encodes or expresses a component of the nervous system; the introduction of a protein, e.g., a protein which is a component of the immune system.

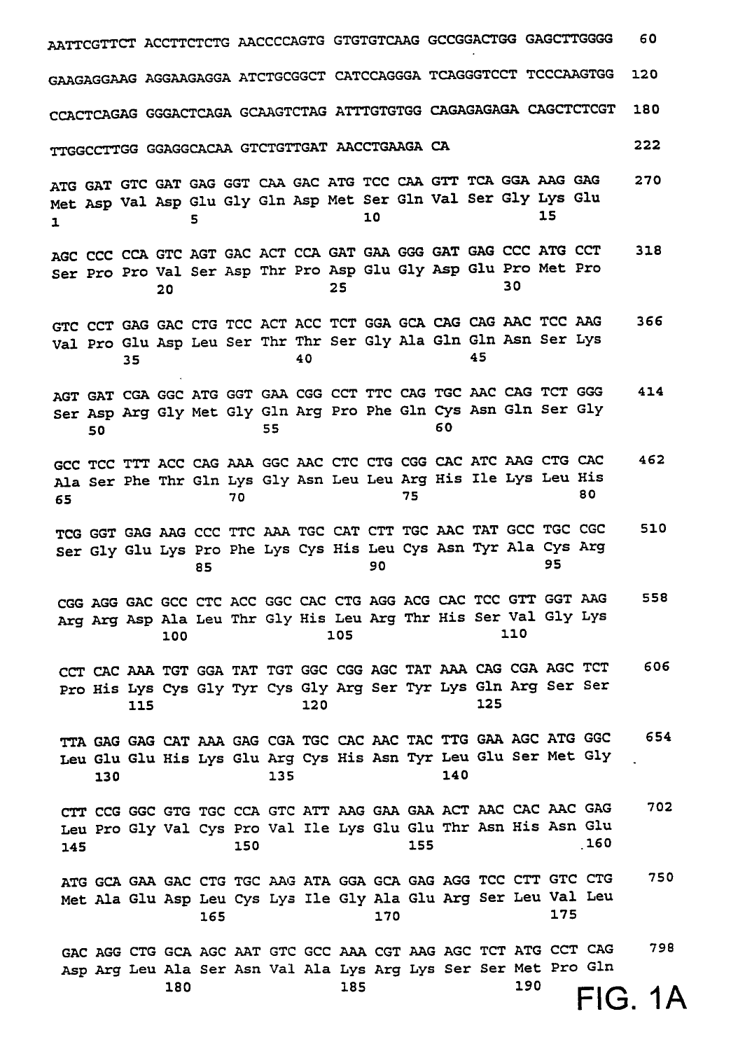

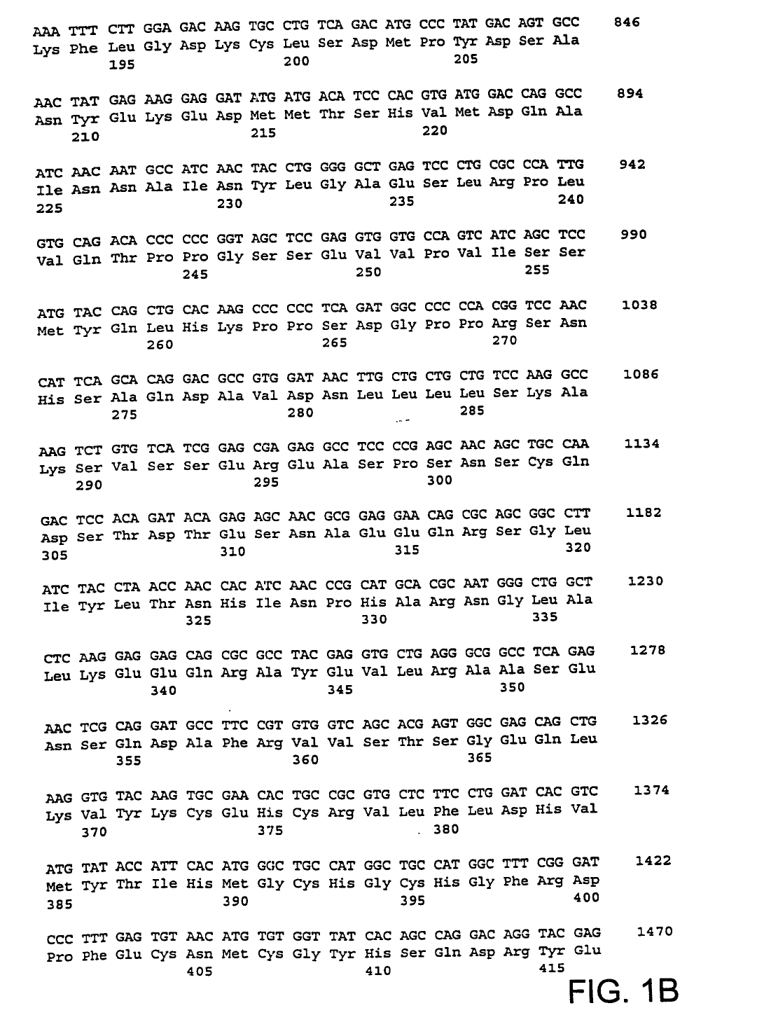

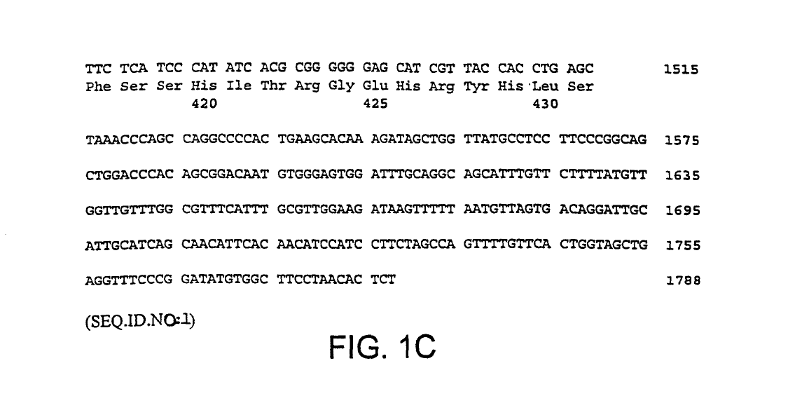

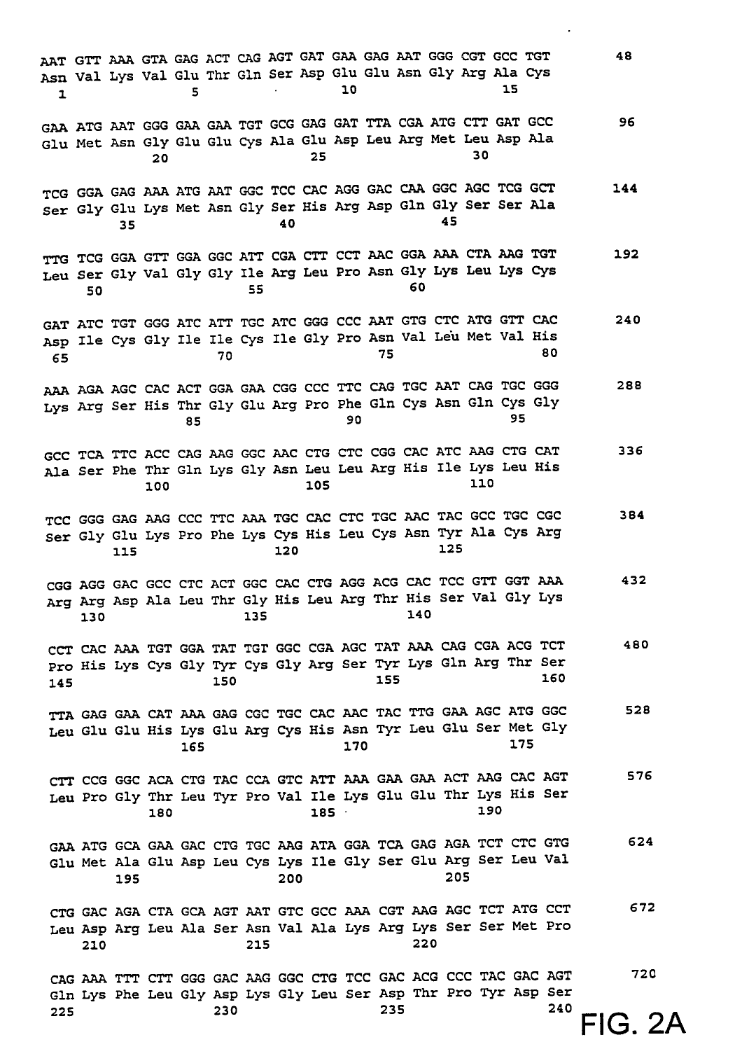

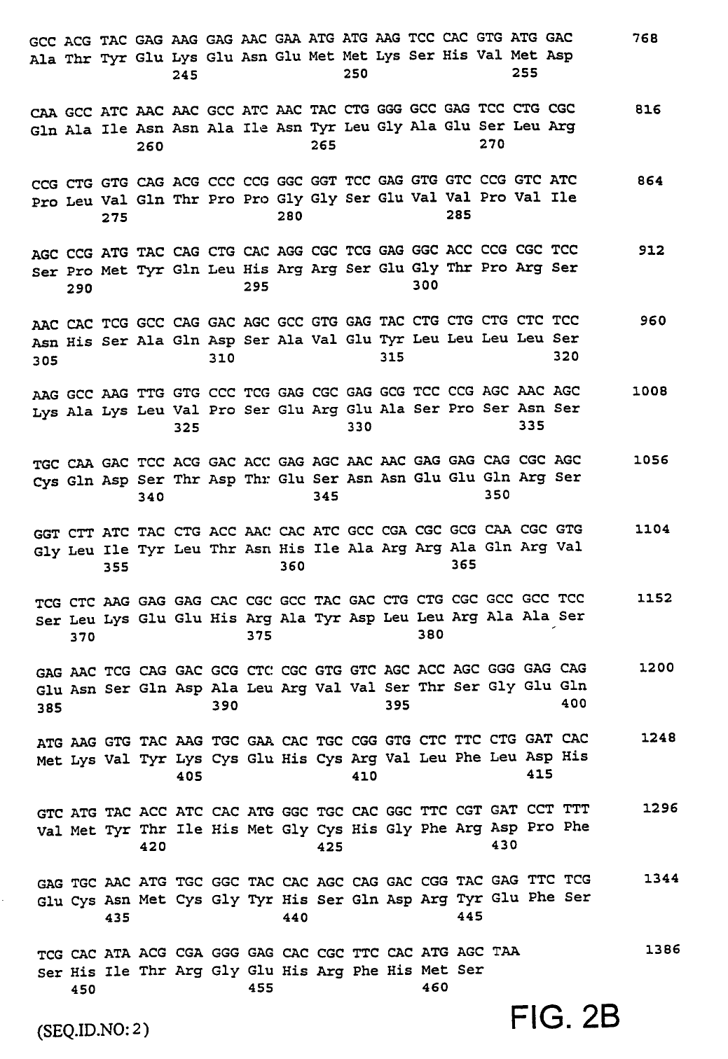

The term “Ikaros” as used herein to refer to a gene, a transgene, or a nucleic acid, refers to a nucleic acid sequence which is at least about 50%, preferably at least about 60%, more preferably at least about 70%, yet more preferably at least about 80%, most preferably at least about 90%-100% homologous with a naturally occurring Ikaros gene or portion thereof, e.g., with the nucleic acid sequence of human Ikaros as shown in SEQ ID NO:2 (FIG. 2 ) or of mouse Ikaros as shown in SEQ ID NO:1 (FIG. 1).

As used herein, the term “transgene” refers to a nucleic acid sequence (encoding, e.g., one or more Ikaros proteins), which is inserted by artifice into a cell. The transgene can become part of the genome of an animal which develops in whole or in part from that cell. If the transgene is integrated into the genome it results in a change in the nucleic acid sequence of the genome into which it is inserted. A transgene can be partly or entirely species-heterologous, i.e., the transgene, or a portion thereof, can be from a species which is different from the cell into which it is introduced. A transgene can be partly or entirely species-homologous, i.e., the transgene, or a portion thereof, can be from the same species as is the cell into which it is introduced. If a transgene is homologous (in the sequence sense or in the species-homologous sense) to an endogenous gene of the cell into which it is introduced, then the transgene, preferably, has one or more of the following characteristics: it is designed for insertion, or is inserted, into the cell's genome in such a way as to alter the sequence of the genome of the cell into which it is inserted (e.g., it is inserted at a location which differs from that of the endogenous gene or its insertion results in a change in the sequence of the endogenous endogenous gene); it includes a mutation, e.g., a mutation which results in misexpression of the transgene; by virtue of its insertion, it can result in misexpression of the gene into which it is inserted, e.g., the insertion can result in a knockout of the gene into which it is inserted. A transgene can include one or more transcriptional regulatory sequences and any other nucleic acid sequences, such as introns, that may be necessary for a desired level or pattern of expression of a selected nucleic acid, all operably linked to the selected nucleic acid. The transgene can include an enhancer sequence. The transgene is typically introduced into the animal, or an ancestor of the animal, at a prenatal, e.g., an embryonic stage.

As used herein, an Ikaros transgene, is a transgene which includes all or part of an Ikaros coding sequence or regulatory sequence. Included are transgenes: which upon insertion result in the misexpression of an endogenous Ikaros gene; which upon insertion results in an additional copy of an Ikaros gene in the cell; which upon insertion place a non-Ikaros gene under the control of an Ikaros regulatory region. Also included are transgenes: which include a copy of the Ikaros gene having a mutation, e.g., a deletion or other mutation which results in misexpression of the transgene (as compared with wild type); which include a functional copy of an Ikaros gene (i.e., a sequence having at least 5% of a wild type activity, e.g., the ability to support the development of T, B, or NK cells); which include a functional (i.e., having at least 5% of a wild type activity, e.g., at least 5% of a wild type level of transcription) or nonfunctional (i.e., having less than 5% of a wild type activity, e.g., less than a 5% of a wild type level of transcription) Ikaros regulatory region which can (optionally) be operably linked to a nucleic acid sequence which encodes a wild type or mutant Ikaros gene product or, a gene product other than an Ikaros gene product, e.g., a reporter gene, a toxin gene, or a gene which is to be expressed in a tissue or at a developmental stage at which Ikaros is expressed. Preferably, the transgene includes at least 10, 20, 30, 40, 50, 100, 200, 500, 1,000, or 2,000 base pairs which have at least 50, 60, 70, 30 80, 90, 95, or 99 % homology with a naturally occurring Ikaros sequence.

As used herein, the term “transgenic cell” refers to a cell containing a transgene.

As used herein, a “transgenic animal” is any animal, e.g., a non-human mammal, e.g., a swine, a monkey, a goat, or a rodent, e.g., a mouse, in which one or more, and preferably essentially all, of the cells of the animal include a transgene. The transgene is introduced into the cell, directly or indirectly by introduction into a precursor of the cell, by way of deliberate genetic manipulation, such as by microinjection or by infection with a recombinant virus. The term genetic manipulation is directed to the introduction of a recombinant DNA molecule. This molecule may be integrated within a chromosome, or it may be extrachromosomally replicating DNA.

The “transgenic animals” of the invention are preferably produced by introducing “transgenes” into the germline of an animal. Embryonal target cells at various developmental stages can be used to introduce transgenes. Different methods are used depending on the stage of development of the embryonal target cell. The zygote is the best target for microinjection. In the mouse, the male pronucleus reaches the size of approximately 20 micrometers in diameter which allows reproducible injection of 1-2p1 of DNA solution. The use of zygotes as a target for gene transfer has a major advantage in that in most cases the injected DNA will be incorporated into the host gene before the first cleavage (Brinster et al. (1985) Proc. Natl. Acad. Sci. USA 82:4438-4442). As a consequence, all cells of the transgenic mammal will carry the incorporated transgene. This will in general also be reflected in the efficient transmission of the transgene to offspring of the founder since 50% of the germ cells will harbor the transgene. Microinjection of zygotes is the preferred method for incorporating transgenes in practicing the invention.

Retroviral infection can also be used to introduce transgene into a mammal. The developing mammalian embryo can be cultured in vitro to the blastocyst stage. During this time, the blastomeres can be targets for retroviral infection (Jaenich, R. (1976) Proc. Natl. Acad. Sci. USA 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) Proc. Natl. Acad. Sci. USA 82:6927-6931; Van der Putten et al. (1985) Proc. Natl. Acad. Sci. USA 82:6148-6152). Transfection is easily and efficiently obtained by culturing the blastomeres on a monolayer of virus-producing cells (Van der Putten et al. (1985) Proc. Natl. Acad. Sci. USA 82:6148-6152; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1985) Proc. Natl. Acad Sci. USA 82:6927-6931).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) Proc. Natl. Acad Sci. USA 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a mammal. The ES cells thereafter colonize the embryo and contribute to the germ line of the resulting chimeric animal. For a review see Jaenisch, R. (1988) Science 240:1468-1474; Sedivy, J. M. and Joyner, A. L. (1992) “Gene Targeting” (W.H. Freeman and Company, New York) 123-142.

For construction of transgenic mice, procedures for embryo manipulation and microinjection are described in, for example, Manipulating the Mouse Embryo, (Cold Spring Harbor Laboratory Press, Cold Spring Harbor, N.Y., 1986). Similar methods are used for production of other transgenic animals. In an exemplary embodiment, mouse zygotes are collected from six-week old females that have been superovulated with pregnant mares serum (PMS) followed 48 hours later with human chorionic gonadotropin. Primed females are placed with males and checked for vaginal plugs on the following morning. Pseudopregnant females are selected for estrus, placed with proven sterile vasectomized males and used as recipients. Zygotes are collected and cumulus cells removed. Pronuclear embryos are recovered from female mice mated to males. Females are treated with pregnant mare serum, PMS, to induce follicular growth and human chorionic gonadotropin, hCG, to induce ovulation. Embryos are recovered in a Dulbecco's modified phosphate buffered saline (DPBS) and maintained in Dulbecco's modified essential medium (DMEM) supplemented with 10% fetal bovine serum.

Microinjection of an Ikaros transgene encoding can be performed using standard micromanipulators attached to a microscope. For instance, embryos are typically held in 100 microliter drops of DPBS under oil while being microinjected. DNA solution is microinjected into the male pronucleus. Successful injection is monitored by swelling of the pronucleus. Immediately after injection embryos are transferred to recipient females, e.g. mature mice mated to vasectomized male mice. In a general protocol, recipient females are anesthetized, paralumbar incisions are made to expose the oviducts, and the embryos are transformed into the ampullary region of the oviducts. The body wall is sutured and the skin closed with wound clips.

Transgenic animals can be identified after birth by standard protocols. For instance, at three weeks of age, about 2-3 cm long tail samples are excised for DNA analysis. The tail samples are digested by incubating overnight at 55° C. in the presence of 0.7 ml 50 mM Tris, pH 8.0, 100 mM EDTA, 0.5% SDS and 350 mg of proteinase K. The digested material is extracted once with equal volume of phenol and once with equal volume of phenol:chloroform (1:1 mixture). The supernatants are mixed with 70 ml 3M sodium acetate (pH 6.0) and the nucleic acid precipitated by adding equal volume of 100% ethanol. The precipitate is collected by centrifugation, washed once with 70% ethanol, dried and dissolved in 100 ml TE buffer (10 mM Tris, pH 8.0 and 1 mM EDTA). The DNA is then cut with BamHI and BglII or EcoRI (or other frequent DNA cutter), electrophoresed on 1% agarose gels, blotted onto nitrocellulose paper and hybridized with labeled primers under very stringent conditions in order to discern between wild-type and mutant receptor genes. Alternatively, a ligation chain reaction (LCR) (see, e.g., Landegran et al. (1988) Science 241:1077-1080; and Nakazawa et al. (1944) Proc. Natl. Acad. Sci. USA 91:360-364), which is useful for detecting point mutations, can be used to determine the presence of the transgene in the neonate.

The resulting transgenic mice or founders can be bred and the offspring analyzed to establish lines from the founders that express the transgene. In the transgenic animals, multiple tissues can be screened to observe for endothelial cell and parenchymal cell expression. RNA studies in the various transgenic mouse lines will allow evaluation of independence of the integration site to expression levels of the transgene.

Mis-expression, as used herein, refers to a non-wild type pattern of gene expression. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of the tissue specificity of expression, e.g., increased or decreased expression (as compared with wild type) in a predetermined cell type or tissue type; a pattern of expression that differs from wild type in terms of the size, amino acid sequence, post-translational modification, or a biological activity of an Ikaros gene product; a pattern of expression that differs from wild type in terms of the effect of an environmental stimulus or extracellullar stimulus on expression of the gene, e.g., a pattern of increased or decreased expression (as compared with wild type) in the presence of an increase or decrease in the strength of the stimulus; or a pattern of isoform expression which differs from wild type.

An Ikaros-responsive control element, as used herein is a region of DNA which, when present upstream or downstream from a gene, results in regulation, e.g., increased transcription of the gene in the presence of an Ikaros protein.

Purified DNA is DNA that is not immediately contiguous with both of the coding sequences with which it is immediately contiguous (i.e., one at the 5′ end and one at the 3′ end) in the naturally occurring genome of the organism from which the DNA of the invention is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA or a genomic DNA fragment produced by PCR or restriction endonuclease treatment) independent of other DNA sequences. It also includes a recombinant DNA which is part of a hybrid gene encoding additional polypeptide sequence.

Homologous refers to the sequence similarity between two polypeptide molecules or between two nucleic acid molecules. When a position in both of the two compared sequences is occupied by the same base or amino acid monomeric subunit, e.g., if a position in each of two DNA molecules is occupied by adenine, then the molecules are homologous at that position. The homology between two sequences is a function of the number of matching or homologous positions shared by the two sequences. For example, 6 of 10, of the positions in two sequences are matched or homologous then the two sequences are 60% homologous. By way of example, the DNA sequences ATTGCC and TATGGC share 50% homology.

The terms peptide, protein, and polypeptide are used interchangeably herein.

A peptide has Ikaros activity if it has one or more of the following properties: the ability to stimulate transcription of a DNA sequence under the control any of a δA element, an NFKB element, or one of the Ikaros binding oligonucleotide consensus sequences disclosed herein; the ability to bind to any of a δA element, an NFKB element, or one of the Ikaros binding oligonucleotide consensus sequences disclosed herein; or the ability to competitively inhibit the binding of a naturally occurring Ikaros isoform to any of a δA element, an NFKB element, or one of the Ikaros binding oligonucleotide consensus sequences disclosed herein. An Ikaros peptide is a peptide with Ikaros activity.

“Ikaros antagonists”, as used herein, refers to Ikaros isoforms arising naturally or by mutagenesis (including in vitro shuffling) which can inhibit at least one biological activity of a naturally occurring Ikaros protein. In preferred embodiments, the Ikaros antagonist is an inhibitor of: Ikaros-mediated transcriptional activation, e.g. it is a competitive inhibitor of Ikaros binding to Ikaros responsive elements, such as IK-BS1, IK-BS2, IK-BS4, IK-BS5, IK-BS6, IK-BS7, IK-BS8, or IK-BS9; or it is an inhibitor of protein-protein interactions of transcriptional complexes formed with naturally occurring Ikaros isoforms.

As used herein, the term “exon”, refers to those gene (e.g. DNA) sequences which are transcribed and processed to form mature messenger RNA (mRNA) encoding an Ikaros protein, or portion thereof, e.g. Ikaros coding sequences, and which, at the chromosomal level, are interrupted by intron sequences. Exemplary exons of the subject Ikaros proteins and genes include: with reference to SEQ ID NO:4 (mIk-1), the nucleotide sequence encoding exon 1/2 (E1/2) corresponding to Met-1 through Met-53; the nucleotide sequence encoding exon 3 (E3) corresponding to Ala-54 through Thr-140; the nucleotide sequence encoding exon 4(E4) corresponding to Gly-141 through Ser-196; the nucleotide sequence encoding exon 5 (E5) corresponding to Val-197 through Pro-237; the nucleotide sequence encoding exon 6 (6) corresponding to Val-238 through Leu-282; the nucleotide sequence encoding exon 7 (E7) corresponding to Gly-283 through Ser-518; with reference to SEQ ID NO:2 (hIk-1), the nucleotide sequence encoding exon 3 (E3) corresponding to Asn-1 through Thr-85; the nucleotide sequence encoding exon 4 (E4) corresponding to Gly-86 through Ser-141; the nucleotide sequence encoding exon 5 (E5) corresponding to Val-142 through Pro-183; the nucleotide sequence encoding exon 6 (6) corresponding to Val- 184 through Leu-228; the nucleotide sequence encoding exon 7 (E7) corresponding to Gly-229 through Ser-461. The term “intron” refers to a DNA sequence present in a given Ikaros gene which is not translated into protein and is generally found between exons. The term “gene” refers to a region of chromosomal DNA which contains DNA sequences encoding an Ikaros protein, including both exon and intron sequences. A “recombinant gene” refers to nucleic acid encoding an Ikaros protein and comprising Ikaros exon sequence, though it may optionally include intron sequences which are either derived from a chromosomal Ikaros gene or from an unrelated chromosomal gene. An exemplary recombinant gene is a nucleic acids having a sequence represented by any of SEQ ID NOS:1-7 or 13.

The term “Ikaros responsive element” or “IK-RE”, refers to nucleic acid sequences which, when placed in proximity of a gene, act as transcriptional regulatory elements which control the level of transcription of the gene in an Ikaros protein-dependent manner. Exemplary IK-RE, as described below, includes IK-BS1, IK-BS2, IK-BS4, IK-BS5, IK-BS6, IK-BS7, IK-BS8, or IK-BS9.

Ikaros: A Master Regulator of Hemopoietic Differentiation

The Ikaros gene is described briefly here. A more detailed treatment can be found in the copending U.S. patent application referred to above. A hemopoietic stem cell in the appropriate microenvironment will commit and differentiate into one of many cell lineages. Signal transduction molecules and transcription factors operating at distinct check points in this developmental pathway will specify the cell fate of these early progenitors. Such molecules are viewed as master regulators in development but also serve as markers for the relatively poorly defined stages of early hemopoiesis.

In search of a lymphoid restricted transcriptional enhancer, in control of gene expression in early T cells, the Ikaros gene family was isolated, which encode zinc finger DNA binding proteins. In the early embryo, the Ikaros gene is expressed in the hemopoietic liver but from mid to late gestation becomes restricted to the thymus. The only other embryonic site with Ikaros mRNA is a small area in the corpus striatum. In the adult, the Ikaros mRNA is detected only in the thymus and in the spleen (Georgopoulos, K. et al. (1992) Science 258:808). The Ikaros gene functions as a transcriptional enhancer when ectopically expressed in non lymphoid cells.

The Ikaros gene plays an important role in early lymphocyte and T cell differentiation. The Ikaros gene is abundantly expressed at early embryonic hemopoietic sites is later on restricted in the developing thymus. The thymus together with the spleen is the prime sites of expression in the adult. This highly enriched expression of the Ikaros gene was also found in early and mature primary T cells and cell lines. This restricted pattern of expression of the Ikaros gene at sites where embryonic and adult T cell progenitors originate together with the ability of the encoded protein to activate transcription from the regulatory domain of an early T cell differentiation antigen supported a determining role in T cell specification.

Differential splicing at the Ikaros genomic locus generates at least five transcripts (Ik-1, Ik-2, Ik-3, Ik-4 and Ik-5) that encode proteins with distinct DNA binding domains. A high level of conservation was found between the human and mouse homologs of the Ikaros gene. The human and mouse Ikaros proteins exhibit nearly 100% identity at their N-terminal zinc finger domain (F1) which was shown to determine the DNA binding specificity of these proteins. In the mouse, differential splicing allows for the distinct combinations of zinc finger modules present in the Ik-1, Ik-2 Ik-3 and Ik-4 isoforms. This differential usage of zinc finger modules in the mouse isoforms establishes the basis of their distinct DNA binding properties and abilities to activate transcription. Differential splicing of the exons encoding the zinc finger DNA binding modules is also manifested in the human Ikaros gene and generates at least two isoforms homologues of the mouse Ik-1 and Ik-4.

These Ikaros protein isoforms (IK-1, IK-2, IK-3, IK-4, IK-5) have overlapping but also distinct DNA binding specificity dictated by the differential usage of zinc finger modules at their N-terminus. In the mouse isoforms (hereinafter designated “mIk”), and presumably in the human isoforms (hereinafter designated “hIk”), the core binding site for four of the Ikaros proteins is the GGGA motif but outside this sequence their specificity differs dramatically. The mIK-3 protein shows strong preferences for bases at both the 5′ and 3′ flanking sequences which restricts the number of sites it can bind to. The mIk-1 protein also exhibits strong preference for some of these flanking bases and can bind to wider range of sequences. The mIk-2 protein, the most promiscuous of the three proteins, can bind to sites with just the GGGAa/t motif. Finally, the mIk-4 protein with similar sequences specificity to mIk-1 binds with high affinity only when a second site is in close proximity suggesting cooperative site occupancy by this protein. Given the identity between the human and mouse Ik-I and Ik-4 DNA binding domains, the human isoforms are expected to bind similar sequences to their mouse homologues and regulate transcription in a similar fashion. This extreme species conservation between these two functionally diverse Ikaros isoforms supports an important role for these proteins in lymphocyte transcription. The C-terminal domain shared by all of the mouse and human Ikaros isoforms is also highly conserved. This portion of the Ikaros proteins contains conserved acidic motifs implicated as transcription activation domains.

The embryonic expression pattern and activation potential of the Ikaros isoforms are also markedly distinct. The stronger transcriptional activators, Ik-1 and Ik-2, are found in abundance in the early fetal liver, in the maturing thymus and in a small area in the developing brain, whereas the weak activators, e.g. Ik-3 and Ik-4, are present at significantly lower levels in these tissues during these times. Consequently, Ik-1 and Ik-2 are expected to play a primary role in transcription from sites that can bind all four of the Ikaros proteins. However, in the early embryonic thymus and in the late mid-gestation hemopoietic liver the weak activator Ik-4 is expressed at similar mRNA levels to the Ik-1 and Ik-2 isoforms. The Ik-4 weak activator can bind only to composite sites while Ik-1 and Ik-2 can bind to a range of single and composite sites. The Ik-1 and Ik-2 proteins recruited to composite sites (a fraction of the total protein), during early to mid gestation, will have to compete for binding with the Ik-4 isoform, solely recruited to these sites. Consequently the activity of these composite sites may be primarily controlled by the Ik-4 isoform, a weak transcription activator. Modulation of Ik-4 expression in the developing thymocyte, in combination with steady levels of the Ik-1 and Ik-2 expression may determine the temporal and stage specific expression of T cell differentiation antigens. Low affinity binding sites for these proteins may also become transcriptionally active in the late stages of T cell development when the most potent activators, Ik-1 and Ik-2, accumulate. In the fly embryo the NF-κB/rel homologue Dorsal, a maternal morphogen, engages in interactions with transcriptional factors binding to adjacent sites. These protein-protein interactions determine the activation level and threshold response from low and high affinity binding sites (Jiang et al. (1993) Cell 72:741-752). The transcriptional activity of the Ikaros proteins may be further regulated by such mechanisms in the developing lymphocyte. In addition, the activity of the Ikaros proteins may be under postranslational control operating during both lymphocyte differentiation and activation. It has been shown that concentrations of Ikaros isoforms at different developmental stages confer different reactivites on the various sites.

The transcriptional activity of the mIk-3 and mIk-4 proteins may be further regulated by T cell restricted signals mediating postranslational modifications or by protein -protein interactions. The mIk-4 protein binds NFkB motif in a cooperative fashion and may therefore interact in situ with other members of the Ikaros or of the NFkB family. These protein-protein-DNA complexes may dictate a differential transcriptional outcome.

The differential expression of the Ikaros isoforms during T cell ontogeny, their overlapping but also unique binding specificities and their diverse transcriptional potential may be responsible for the orderly activation of stage specific T cell differentiation markers. Multiple layers of gene expression in developing lymphocytes may be under the control of these Ikaros proteins. Synergistic interactions and/or competition between members of the Ikaros family and other transcription factors in these cells on qualitatively similar and distinct target sites could dictate the complex and ever changing gene expression in the differentiating and activated lymphocyte. This functional dissection of the Ikaros gene strongly suggest that it functions as a master gene in lymphocytes, and an important genetic switch for early hemopoiesis and both B and T cell development.

The Ikaros gene maps to the proximal arm of human chromosome 7 between p11.2 and p13 next to Erbb In the mouse the Ikaros gene maps to the proximal arm of chromosome 11 tightly linked to Erbb. Other genes linked to the Ikaros locus in the mouse are the Leukemia inhibitory factor (Lif) and the oncogene Rel a member of the NFK-B family. All three of the genes linked to the Ikaros gene in the mouse appear to play an important role in the development of the hemopoietic system. The tight linkage between the Erbb and the Ikaros genes on syntenic loci in the mouse and human may be related to their genetic structure and regulation. Nevertheless, no known mutations were mapped to the Ikaros locus in the mouse. However, this does not preclude the importance of the Ikaros gene for the lymphopoietic system. Naturally occurring mutations that affect development of the immune system may not be readily obtained in mice since such mutant animals may only thrive under special care conditions

That the Ikaros gene is a fundamentally important regulator of lymphocyte development is substantiated by analysis of its human homologue. The overall conservation of the Ikaros proteins between mice and man at the genetic level and protein level but also their restricted pattern of expression in the developing lymphocyte, e.g. in maturing T cells, e.g. in maturing B cell, strongly support their participation in the same regulatory pathway across species.

Cloning the Mouse Ikaros Gene

A T cell expression cDNA library from the mature T cell line E14 was constructed into the A ZAP phage vector.

A multimerized oligonucleotide encoding sequence (SEQ ID NO:14) from one of the protein binding sites of the CD38 enhancer was used as a radio labeled probe to screen this expression library for the T cell specific proteins that bind and mediate enhancer function by the southwestern protocol of Singh and McKnight. Four gene encoding DNA binding proteins were isolated. One, the Ikaros gene, encoded a T cell specific protein.

The Sequence of Mouse Ikaros

The sequence of the Ikaros gene was determined using the Sanger dideoxyl sequencing protocol. The derived amino acid sequence was determined using the MAP program of GCG (available from the University of Wisconsin) and Strider sequence analysis programs. FIG. 1 provides the sequence of a mouse Ikaros cDNA (mIk-2) and the derived amino acid sequence encoded thereby (SEQ ID NO:1). Sequence information for other isoforms of mouse Ikaros proteins (and cDNAs) are provided in SEQ ID NO:3 (mIk-3), SEQ ID NO:4 (mIk-1), SEQ ID NO:5 (mIk-4), and SEQ ID NO:6 (mIk-5).

A Mouse Ikaros Protein

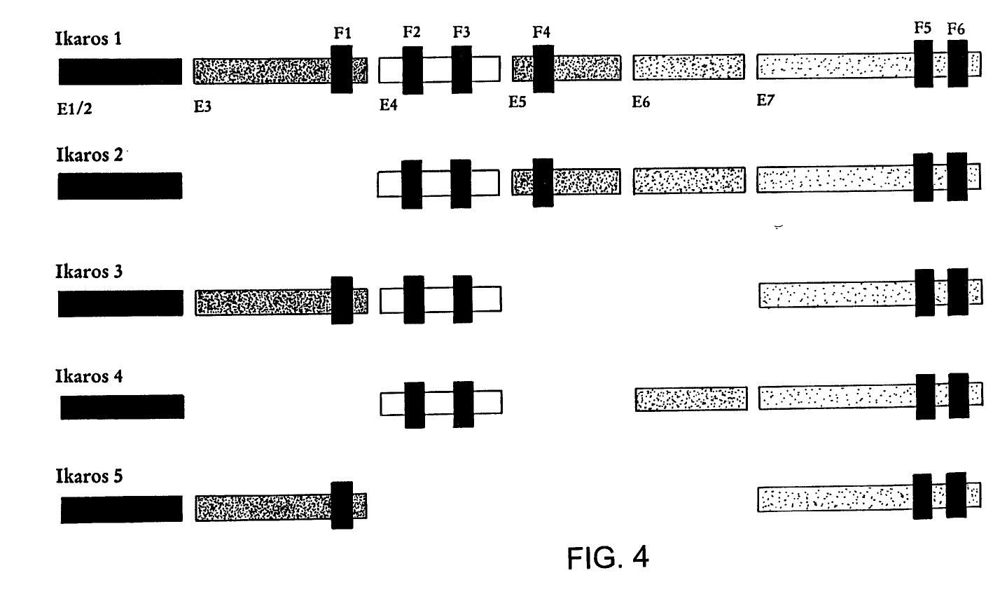

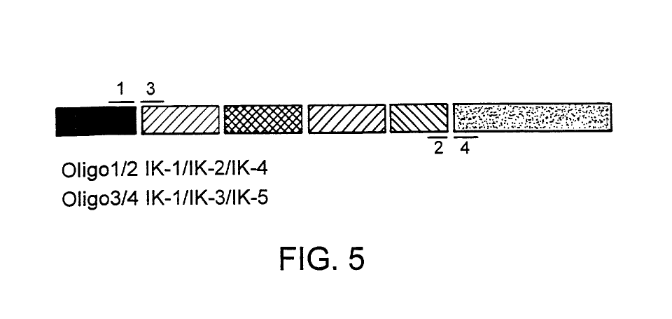

The Ikaros protein shown in FIG. 1 (mIk-2) is comprised of 431 amino acids with five CX₂CX₁₂HX₃H zinc finger motifs organized in two separate clusters. (See also FIG. 4.) The first cluster of three fingers is located 59 amino acids from the initiating methionine, while the second cluster is found at the C terminus of the protein 245 amino acids downstream from the first. Two of the finger modules of this protein deviate from the consensus amino acid composition of the Cys-His family of zinc fingers; finger 3 in the first cluster and finger 5 at the C terminus have four amino acids between the histidine residues. This arrangement of zinc fingers in two widely separated regions is reminiscent of that of the Drosophila segmentation gap gene Hunchback. Similarity searches in the protein database revealed a 43% identity between the second finger cluster of Ikaros and Hunchback at the C terminus of these molecules. This similarity at the C terminus of these proteins and the similar arrangement of their finger domains raises the possibility that these proteins are evolutionary related and belong to a subfamily of zinc finger proteins conserved across species.

Ikaros Isoforms

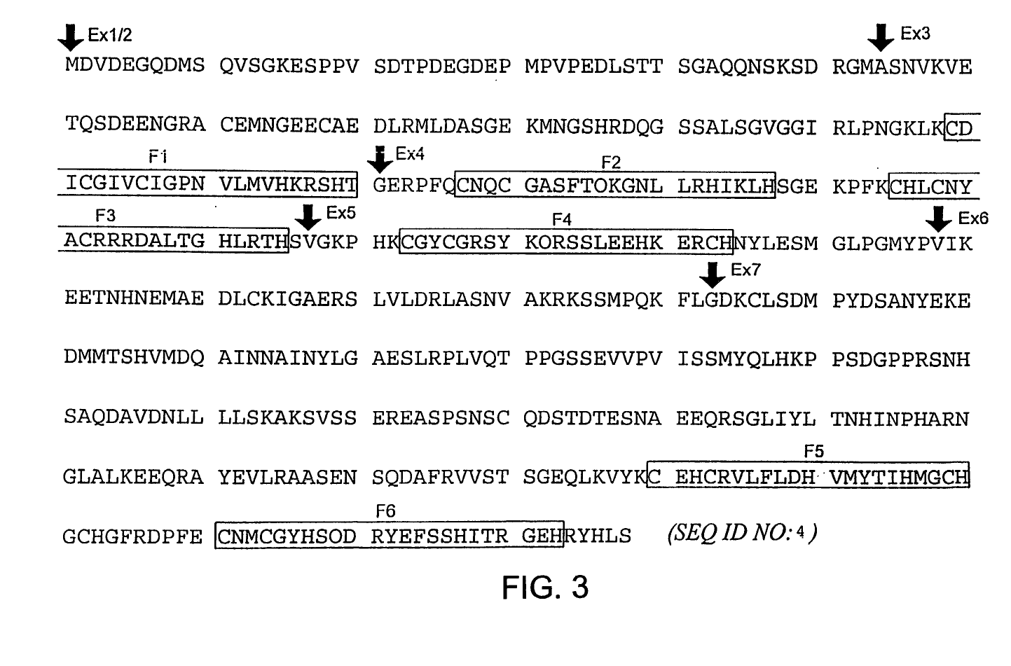

In addition to the cDNA corresponding to mIk-2, four other cDNAs produced by differential splicing at the Ikaros genomic locus were cloned. These isoform encoding cDNAs were identified using a 300 bp fragment from the 3′ of the previously characterized Ikaros cDNA (mIk-2, FIG. 1). As shown in FIG. 3 and 4, each isoform is derived from three or more of six exons, referred to as E1/2, E3, E4, E5, E6 and E7. All five cDNAs share exons E1/2 and E7 encoding respectively for the N-53 and C-terminal 236 amino acid domains. These five cDNAs consist of different combinations of exons E3-6 encoding the N-terminal zinc finger domain. The mIk-1 cDNA (SEQ ID NO:4) encodes a 57.5 kD protein with four zinc fingers at its N-terminus and two at its C-terminus and has the strongest similarity to the Drosophila segmentation protein Hunchback (Zinc fingers are indicated as F1, F2+F3, F4, and F5+F6 in FIG. 4). The mIk-2 (SEQ ID NO:1) and mIk-3 (SEQ ID NO:3) cDNAs encode 48 kd proteins with overlapping but different combinations of zinc fingers. The mIk-3 isoform contains fingers 1, 2, 3 while mIk-2 contains fingers 2, 3 and 4. The 43.5 kD mIk-4 protein (SEQ ID NO:5) has two fingers at its N-terminus also present in mIk-1 and mIk-2. The mIk-5 cDNA (SEQ ID NO:6) encodes a 42 kd protein with only one N-terminal finger shared by mIk-1 and mIk-3. This differential usage of the zinc finger modules by the Ikaros proteins support an overlapping but differential DNA binding specificity.

cDNA cloning of isoforms was performed as follows. A cDNA library made from the T cell line EL4 in λZAP was screened at high stringency with a 300 bp fragment from the 3′ of the previously described Ikaros cDNA (isoform 2). Positive clones were characterized by sequencing using an antisense primer from the 5′ of exon 7.

Cloning of the Human Ikaros Gene

A DNA fragment derived from the shared 3′ coding region of the mouse Ikaros cDNAs was used as a probe to screen for human Ikaros homologs. This DNA fragment, which encodes the C-terminal part of the Ikaros proteins, is believed to be essential for their activity and does not exhibit significant sequence similarities with other DNA binding proteins. A cDNA library from the human T cell line Jurkat was screened at high stringency and 9 partial cDNAs were isolated. The most full length cDNA and its deduced amino acid sequence are shown in FIG. 2 (SEQ ID NO:2). This cDNA encodes a protein homologous to the mouse Ik-1 isoform, the largest of the mouse Ikaros proteins comprised of all the translated exons. A high degree of conservation was detected between the human and the mouse Ik-1 isoforms both at the DNA and the protein levels. The portion of the mouse Ik-1 that contains exons 3 through 7 display 89% and 91% identity to its human homologue at the DNA and protein levels respectively However the N-terminal portion of the mouse Ik-1 isoform encoded by exons 1/2 was not found in any of the three human cDNAs. The cDNAs instead display distinct 5′ ends. The lack of conservation in this part of the human and mouse Ikaros proteins suggest that each of their N-terminal portions are probably not functionally significant. The distinct 5′ untranslated sequences present in these human cDNAs are reminiscent of the number of distinct 5′ untranslated sequences present in mouse cDNA products of potential alternate promoter usage.

Of the human cDNAs isolated, only one contained the splicing junction between exons- 4 and -6 found in the mouse Ik-4 isoform. The lower frequency of cloning of human Ik-4 relative to human Ik-1 cDNAs may reflect their relative concentrations in this T cell line. In the mouse, the Ik-1 isoform is found in excess relative to the Ik-4 isoform in the differentiating T cells (A. Molnar et al 1994).

Human Ikaros isoforms were cloned as follows: A human cDNA library made from the mature T cell line Jurkat (Stratagene) was screened with a 150 bp single stranded probe derived from the most 3′ of the IK-1 mouse Ikaros cDNA. From the 8×10⁵ recombinant phages screened, 9 positive clones were obtained. Filters with recombinant phage DNA were incubated overnight in hybridization buffer (7% SDS, 1% BSA, 0.25 Sodium-phosphate pH 6.5 and 0.5 mM EDTA) with 1×10⁶ cpm/ml probe at 65° C. Washes were performed twice in 2×SSC/1%SDS, 0.2×SSC/1%SDS and 0.2×SSC/01%SDS at 65° prior to autoradiogarphy. Positive clones were purified and characterized by dideoxy sequencing.

Expression of the Ikaros Gene in Human Tissues and Cell Lines

Expression of the Ikaros gene was determined in human tissue and cell lines. Two major Ikaros RNA transcripts were detected only in polyA+ RNA from thymus, spleen, and peripheral leukocytes. Very low levels of Ikaros mRNA were also detected in the colon, and probably reflects the resident lymphocyte population in this tissue. The smaller (28S) of the two Ikaros mRNA forms correlates in size with the major Ikaros transcript detected in the mouse, while the larger form correlates in size with a low abundance transcript detected in the mouse upon overexposure of Northern blots. High levels of both of these mRNAs were expressed in the thymus, while the larger form predominated in the spleen. In peripheral leukocytes equal amounts of both transcripts were present, but at 2 fold lower level than in the thymus. These two mRNA species detected in the human may represent products of differential splicing with the larger species containing additional 5′ and/or 3′ non-coding exons. In addition, they may be transcribed from distinct promoters and may be comprised of different combinations of 5′ untranslated exons.

Northern Analysis was carried out as follows: Two Northern blots each containing 2 μgs of poly A+ RNA isolated from human heart, brain, placenta, lung, liver, skeletal muscle kidney, and pancreas (Clontech human blot) and from spleen, thymus, prostate, testis, ovary, small intestine, colon, and peripheral blood leukocytes (Clontech human blot II) were hybridized with a probe (10⁶ cpm /ml in hybridization buffer) made from the 800 bp SacI-EcoRI fragment of hIk- 1 cDNA. A northern blot containing 10 μgs of total RNA prepared from the T cell leukemic lines: CEM, Molt-4, from the acute myelogenous leukemia KG1, the acute monocytic leukemia THP-1, the U937 histiocytic lymphoma, 30 μgs of the T cell line HPB 1 and 2.5 μgs of human thymus.

The Ikaros Protein Isoforms are Conserved Between Mouse and Man

The expression of the Ikaros protein isoforms was examined in human and mouse T cell nuclear extracts by Western blotting. Nuclear extracts from mouse and human fibroblast and epithelial cells were used to determine the specificity of the Ikaros antibody. A number of cross reacting proteins were detected in the nuclear extract from the mouse EL-4 T cell line. Since cDNAs that encode at least five size distinct Ikaros proteins were cloned from this cell line, the proteins detected with the Ikaros antibody are probably Ikaros isoforms expressed in this cell line. In the human T cell line Jurkat, the largest of these proteins was the most abundant form but other smaller proteins were detected at lower abundance. These human T cell nuclear proteins may represent the homologues of the mouse Ik-1, Ik-2, Ik-3 and Ik-4 isoforms in order of decreasing relative concentration. No cross reacting proteins were detected in the nuclear extracts from the CV1 and NIH-3T3 non expressing cell lines, thus confirming the specificity of the detecting antibody

Western analysis of human and mouse nuclear extracts were carried out as follows: 20 μgs of protein, from nuclear extracts prepared from the Ikaros expressing mouse and human T cell lines EL4 and Jurkat, and from the Ikaros non-expressing mouse and monkey fibroblast and kidney epithelial lines NIH-3T3 and CV1, were run on 12% PAGE. Proteins were transferred to a nitrocellulose membrane and were analyzed with a 1:250 dilution of Ikaros antibody raised to the N-terminal portion of the mouse Ik-2 isoform containing exons 1, 3, 4, 5, and 6. The second step was performed using 1:3000 dilution of goat anti-rabbit antibody (BioRAD) conjugated to alkaline phosphatase. Antibody complexes were detected with BCIP and NBT substrates.

The Ikaros Mouse Genomic Locus

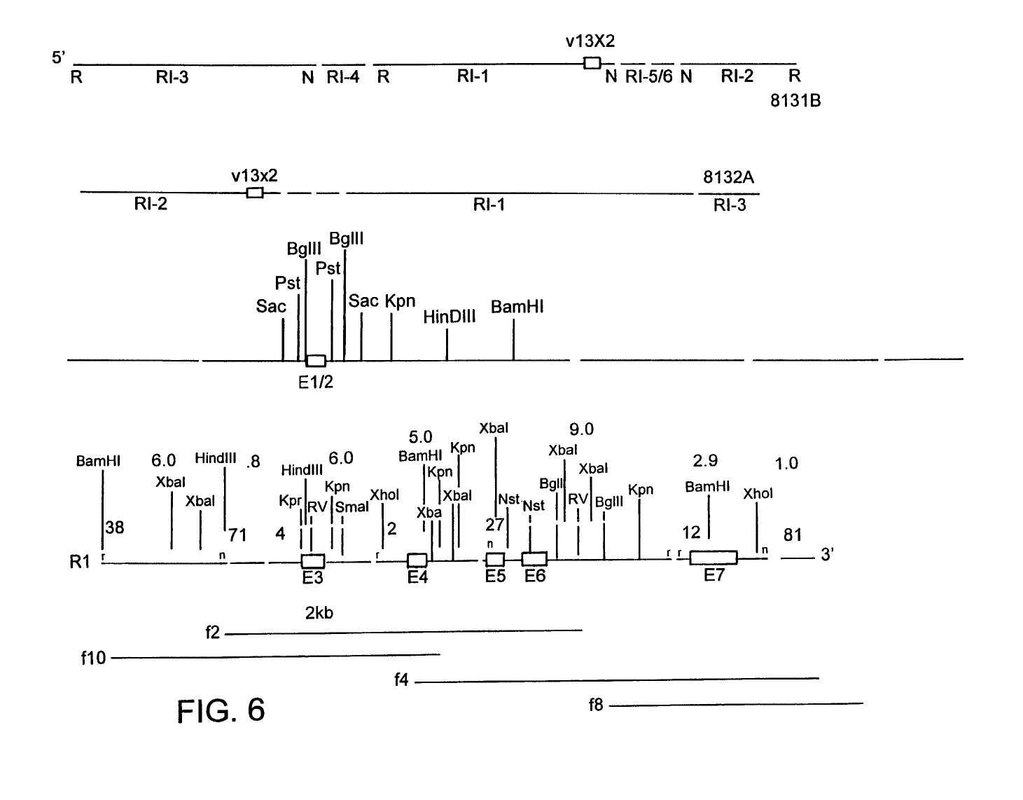

Based on sequence analysis of variant cDNAs, the genomic locus is thought to include about 9-11 exons. Genomic DNAs encompassing most or all of the Ikaros exons present in the genome were isolated by screening a mouse genomic SV129 library made into the λDASH II phage vector using the various Ikaros cDNAs as probes. The Ikaros gene includes at least 80-90 kb of genomic sequence which was isolated as distinct but also overlapping genomic clones. Some of the Ikaros genomic clones are indicated in FIG. 6. The exons are depicted as boxes while the introns as lines. The DNA sequence for: the 5′ boundary (SEQ ID NO:8) and the 3′ boundary (SEQ ID NO:9) of exon E5; the 5′ boundary (SEQ ID NO:10) of exon E3; and the 5′ boundary (SEQ ID NO:11) and the 3′ boundary (SEQ ID NO:12) of exon E7, were determined.

The mouse Ikaros Gene is Located at the Proximal Arm of Chromosome 11

The mouse chromosomal location of Ikaros was determined by interspecific backcross analysis using progeny derived from matings of [(C57BL/6J x F1 X C57BL/6J] mice. This interspecific backcross mapping panel has been typed for over 1300 loci that are well distributed among all the autosomes as well as the X chromosome. C57BL/6J and M spretus DNAs were digested with several enzymes and analyzed by Southern blot hybridization for informative restriction fragment length polymorphisms (RFLPs) using a mouse cDNA fragment as a probe. The 6.5 kb M. Spretus PstI restriction-fragment-length polymorphism (RFLP) was used to follow the segregation of the Ikaros locus in backcross mice. The mapping results indicated that Ikaros is located in the proximal region of mouse chromosome 11 linked to Lif, Erbb and Rel. Although 129 mice were analyzed for every marker, up to 157 mice were typed for some pairs of markers. Each locus was analyzed in pairwise combinations for recombination frequencies using the additional data. The ratios of the total number of mice exhibiting recombinant chromosomes to the total number of mice analyzed for each pair of loci and the most likely gene order are: centromere-Lif-6/167-Ikaros-3/146-Erbb-6/158-Rel. The recombination frequencies [expressed as genetic distances in centiMorgans (cM) +/− the standard error] are-Lif-3.6+/−1.4-Ikaros-2.1+/−1.2-Erbb-3.8+/−1.5-Rel.

The interspecific map of chromosome 11 was composed with a composite mouse linkage map that reports the map location of many uncloned mouse mutations (compiled by M. T. Davisson, T. H. Roderick, A. L. Hillyard, and D. P. Doolittle and provided from GBASE, a computerized database maintained at The Jackson Laboratory, Bar Harbor, Me.). Ikaros mapped in a region of the composite map that lacks mouse mutations with a phenotype that might be expected for an alteration in this locus.

The proximal region of mouse chromosome 11 shares a region of homology with human chromosomes 22, 7 and 2. In particular Erbb has been placed on human 7p12. The tight linkage between Erbb and Ikaros in mouse suggests that Ikaros will reside on 7p as well.

Interspecific backcross progeny were generated by mating (C57BL/6J x M. spretus) F1 females and C57BL/6J males as described (Copeland and Jenkins, 1991). Trends Genet 7:113-118. A total of 205 F2 mice were used to map the Ikaros locus DNA isolation, restriction enzyme digestion, agarose gel electrophoresis, Southern blot transfer and hybridization were performed essentially as described (Jenkins et al. (1982) J. Virol. 43:26-36; and Jenkins et al (1982) J. Virol. 42:379-388). All blots were prepared with Zetabind nylon membrane (AMF-Cuno). The probe, a 350 bp mouse cDNA fragment was labeled with [α-³²P] dCTP using a random prime labeling kit (Amersham); washing was done to a final stringency of 1.0×SSCP, 0.1% SDS, 65° C. A fragment of 8.4 kb was detected in PstI digested C57BL/6J DNA and a fragment of 6.5 kb was detected in PstI digested M. spretus DNA. The presence or absence of the 6.5 kb M. spretus-specific PstI fragment was followed in backcross mice.

A description of the probes and RFLPs for the loci linked to Ikaros including leukemia inhibitory factor (Lif), avian erythroblastosis oncogene B (Erbb) and reticuloendotheliosis oncogene (Rel) has been reported previously (Karl et al. (1993) Mol Cell Biol 10:342-301; Karl et al. (1992) Genetics 131:103-173; and Karl et al. (1992) Science 256:100-102). Recombination distances were calculated using the computer program SPRETUS MADNESS. Gene order was determined by minimizing the number of recombination events required to explain the allele distribution patterns.

The Ikaros Gene Maps Between p11.2-p13 on human chromosome 7

The human chromosome assignment of the Ikaros gene was performed using DNAs prepared from a panel of somatic cell hybrids made between human and rodent. Primers designed after non-conserved sequences at the 3′ end of the human cDNAs were used to distinguish between the human and rodent genes. A 375 bp fragment, as predicted from the human Ik-1 cDNA was amplified from human DNA used as a control and from DNA prepared from the cell hybrid 10791 which contains chromosome 7. The identity of the amplified band was confirmed using a probe derived from this region. To fine map the location of the Ikaros gene a panel of somatic cell hybrids which contained parts of chromosome 7 fused to the rodent genome were analyzed. A hybridizing 10 kb BglII genomic fragment was detected with human genomic DNA. A fragment of similar size was readily detected with DNA from the cell lines Ru Rag 4-13 and 1365 Rag12-9. The former cell line contained the proximal arm of chromosome 7 while the latter contained the distal and part of the proximal up to segment p13. DNA from Rag GN6, a cell line that contains the whole distal arm of chromosome 7 and the proximal arm up to segment p11.2, did not hybridize. Another cell line which contained part of the proximal arm of chromosome 7 from p- to the telomere did not hybridize. This mapping restricts the location of the Ikaros gene between p11.2 and p13, placing it proximate to the Erbb gene locus, as predicted from the mouse.

PCR analysis of somatic cell hybrid DNA prepared from human-mouse-hamster and human-rodent somatic cell hybrids were used for the chromosome assignment of the human Ikaros gene DNAs from the following cell lines were used in PCR reactions h/h human-hamster hybrid h/m: human-mouse hybrid, 1 to 24 respectively 07299-h/h,1082613-h/h, 10253-h/h, 10115-h/h 10114-h/h, 10629-h/h 10791-h/h, 10156B-h/h,10611-h/h, 10926B-h/h,10927A-h/h 10868-h/h, 10898-h/h 10479-h/m 11418-h/m 10567-h/m 10498-h/m 11010-h/h 10449-h/h 10478-h/m 10323-h/m 10888-h/h, 06318B-h/h 06317-h/h 25 human 26 mouse and 27: hamster DNAs were also used in control reactions 100 ngs of these DNAs were used in a PCR reaction together with 150 ngs of primers hIK-1 GGCTGCCACGGCTTCCGTGATCCT (SEQ ID NO:15) and hIk-2: AGCGGTCTGGGGAAACATCTAGGA (SEQ ID NO:16) designed after non-conserved sequences at the 3 min. of the human cDNA. Amplification parameters were: 95° C. for 5 min., 80° C. for 10 min. (with addition of 2.5 units of Taq polymerase), followed by 30 cycles at 93° C. for 1 min., 65° C. for 1 min. and 72° C. 40″, with an additional cycle at 93° C. for 5 min., 65° C. for 2 min. and 72° C. for 7 min. The amplified 375 bp product corresponds to the predicted size from the human cDNA. Fragment identity was confirmed by Southern hybridization with a probe derived from this region.

Fine mapping on human chromosome was further obtained by preparing 7 DNAs from a chromosome 7 hybrid panel which was used either in PCR amplification reactions with the primers described above, or in Southern analysis. The human chromosome 7 content of the hybrid cell lines used were 1365 Rag 12-9: 7qter-p13; Rag GN6:7qter-pl 1.2; Ru Rag 4-13: 7cen-pter (Vortkamp et. al. (1991) Genomics 11:737-743). For Southern blot analysis, 5 μg of human DNA and 10 μgs of hybrid and mouse DNA digested with BglII were hybridized with a 375 bp fragment contained within the hIk-1 and hIk-2 primers.

Generation of Transgenic Mice: Targeted Deletion of the DNA Binding Domain (Exons 3 and 4) in the Ikaros Gene (Mutation 2) and the Generation of Ikaros +/− and −/− Mutant Mice

Cloning of the Ikaros gene, recombination constructs and targeting of embryonic stem (ES) cells.

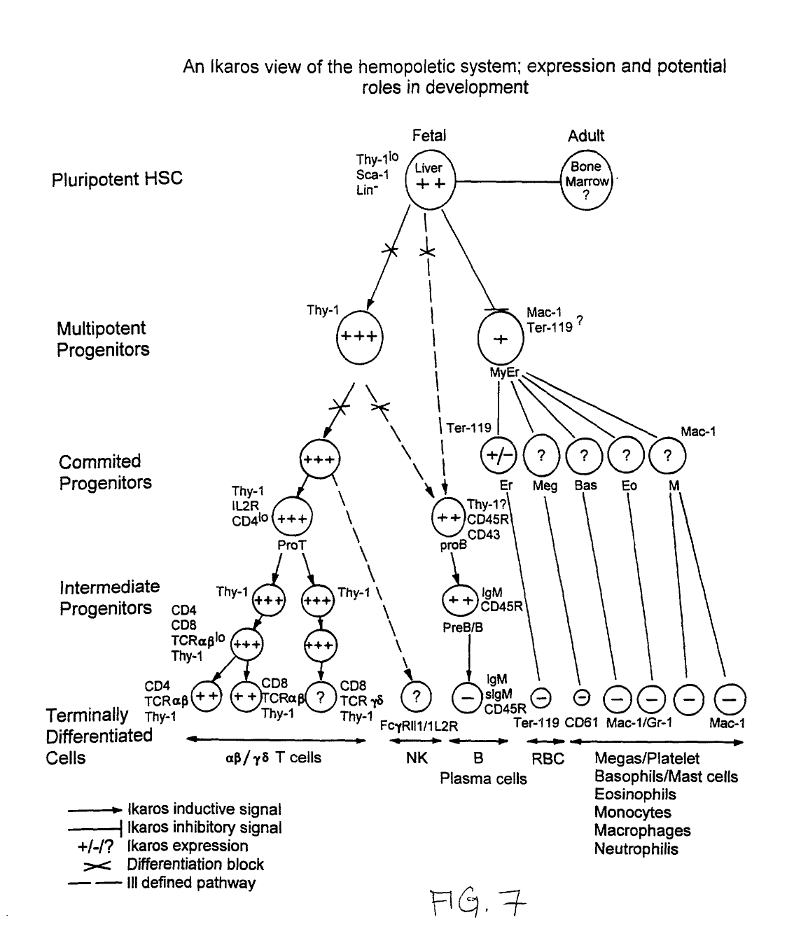

A liver genomic library made from SV129 mouse liver DNA into the phage vector λ DASH II was screened with probes derived from the mouse Ikaros cDNA Ikaros-1 (Molnar, et al., 1994). Overlapping genomic clones were isolated that cover a region of 100 kb containing at least 6 translated exons. The recombination vector was constructed with Ikaros genomic fragments and the neomycin and thymidine kinase expression cassettes (Li, E. et al. (1992) Cell 69:915-926) using standard molecular biology protocols. 25 μgs of the recombination vector were electroporated into 1×10⁷ J1 embryonic stem cells maintained on subconfluent embryonic fibroblasts. Transfected ES cells were originally plated on embryonic fibroblasts and grown without selection. 20 hrs later media containing G418(400 μgs/ml) and 48hrs G418 and FIAU (0.2 μM Bristol Myers) were added. Cells were fed every two days, colonies were monitored for their undifferentiated morphology and picked between seven and nine days after plating. After DNA analysis, a number of ES cell clones with legitimate recombination events were placed back into culture and the ones which displayed undifferentiated properties were passaged once more before they were injected into a day 3.5 C57BL/6 or Balb/c blastocyst. Chimeric blastocysts were then injected in pseudo-pregnant foster mothers. Chimeric animals were born 18 days later and the ones that were more than 40% agouti were bred against background. Female and male F1 mice with germ line transmission of the Ikaros mutation were bred to homozygocity. The genotype of F1 and F2 mice was determined by Southern and by PCR analysis of tail DNA using either probe A as shown in FIG. 8A or appropriate primers designed from the neomycin (Neo1) and the Ikaros genes (Ex3F and Ex3R).

| | |
|---|---|
| Ex3F:AGT AAT GTT AAA GTA GAG ACT CAG | (SEQ ID |
| | NO:17); |
| Ex3R:GTA TGA CTT CTT TTG TGA ACC ATG | (SEQ ID |
| | NO:18); |
| Neol:CCA GCC TCT GAG CCC AGA AAG CGA | (SEQ ID |
| | NO:19) |

Given the extensive differential splicing of Ikaros transcripts (Molnar, A. et al., (1994)), the multiple transcription initiation sites and the size and complexity of this genomic locus, a recombination vector was designed to replace an 8.5 kb genomic fragment containing part of exon 3 and exon 4 with the neomycin cassette. Probe A, which was derived from a region outside the recombination locus was used to screen for homologous recombination events. This mutation deletes zinc fingers −1, −2, and −3, responsible for mediating the sequence specific DNA binding of the Ikaros proteins. This mutation should prevent the Ikaros proteins from binding DNA and activating transcription (Molnar, et al., 1994).

This recombination vector was targeted in the embryonic stem (ES) cell line J1 (Li, E. et al. (1992) Cell 69:915-926). 300 neomycin and FIAU resistant ES cell colonies were picked and expanded. DNA was prepared and analyzed by Southern blotting using DNA probes from outside the homologous recombination area. Analysis of genomic DNA from 12 selected ES cell clones was performed. A 12.5 kB and a 10.5 kB BamHI genomic fragments from the wild type and the targeted Ikaros alleles respectively hybridized to probe A. Single integration events were scored using a probe derived from the neomycin gene. The homologous recombination frequency among the ES cell clones analyzed was 1:10. Two ES cell lines with legitimate homologous recombination events and with undifferentiated growth properties were passaged another time and were then injected into day 3.5 blastocysts ES cells whose DNA analysis is shown in lanes 4 and 9. Two distinct ES cell lines heterozygous for this mutation were used in separate blastocyst injections to rule out phenotypes that result from cell line mutations. To explore potential phenotype variability on different genetic backgrounds the mutant ES cells were injected in blastocysts from C57BL/6 and Balb/c mice. The chimeric blastocysts were reimplanted in pseudo-pregnant mice which gave birth to chimeric animals. Chimeras which were more than 40% agouti (SV129 positive) were bred against their host background. Male and female F1 progeny with germ line transmission were bred against each other. F2 litters were scored for wild type, heterozygous and homozygous pups. Southern analysis of tail DNAs from a 2-week old F2 litter which revealed the occurrence of homozygous offspring at the expected Mendelian frequency.

Characterization of Transgenic Mice Heterozygous for the DNA-binding Defective Transgene

Ikaros −/+ transgenic Animals Develop Lymphomas

Animals heterozygous for the Ikaros mutations develop lymphoproliferations in the thymus, spleen, and lymph nodes. The lymphoid organs become significantly enlarged, the spleen reaches the size of 4.5×1.3×0.6 cm. The thymus can range from moderately enlarged to occupying the whole thoracic cavity and the cervical and auxiliary lymph nodes can reach the size of 1 cm. The penetrance of lymphoproliferation of 100%. Most animals develop this syndrome around 2-3 months and do not survive past the fifth month of age. Microscopic examination of blood smears from these animals revealed large nucleated blast like cells with azurophilic cytoplasm and prominent nucleoli. These large nucleated cells predominate leukocytes in the blood smear of all animals. The leukocyte count in the blood of these animals is often 6 times the number of that in the blood of their wild type littermates.

The cell populations of the spleen, the thymus, the lymph nodes and the bone marrow in the affected animals were analyzed with antibodies to T, B, myeloid and erythroid differentiation antigens by FACS. The majority of the cells analyzed were positive for Thy 1, CD5, TCR, CD25, CD18 antigens which demarcate mature but also activated T cells. This population was predominant in all four lymphoid tissues suggesting expansion of a T cell in all lymphomas. Cells obtained from these animals can be propagated in tissue culture in the presence of IL-2.

Preliminary cDNA and Northern analysis of these cells revealed three separate splicing events which join exon 2 to exon 5 and exon 7. These mutant mRNAs can generate proteins lacking the DNA binding domain (deleted exons 3 and 4) but containing their C-terminal part, similar or identical to the naturally occurring isoforms IL-5 and IK-6.

Characterization of Transgenic Animals Homozygous for the DNA-binding Defective Transgene

Ikaros −/− Mutant Mice are Born but Fail to Thrive

Mice homozygous for the Ikaros mutation 2 were born with the expected Mendelian frequency indicating that the mutation does not affect their survival in utero. At birth homozygous, heterozygous and wild type littermates were indistinguishable. One week past birth, however, homozygous pups were identifiable by their smaller size. This size difference escalates during the third and fourth weeks of their lives. The size of homozygous animals varied from ⅓ to ⅔ of that of their wild type littermates and most of them displayed a matted coat appearance.

No morphological and hemopoietic cell differences were detected between wild type and heterozygous pups. A large majority of the Ikaros −/− mutant mice (approximately 95%) died between the first and third week of their life. A large proportion of these deaths were associated with cannibalism by the mothers. The mortality rate was higher on the C57BL/6 mixed background where mothers were less tolerant of defective pups. Mutant animals survived better in smaller litters suggesting that competition in a larger litter may escalate the death rate.

Analysis of homozygous mice derived from the two distinct ES cell clones verified that the phenotype observed was due to the mutation in the Ikaros gene. Ikaros −/− mutant mice derived from either ES cell clones were identical in terms of their growth, survival, hemopoietic populations and disease contraction. Animals were studied from several days to 12 weeks past birth on the SV129xBalbc, SV129xC57 and SV129 backgrounds. Normal looking and severely growth retarded mutant mice were examined. Their hemopoietic system was extensively studied. Finally their inability to thrive and cause of death was investigated. The overall hemopoietic phenotype and disease contraction in homozygous animals described in the following sections was the same on all three genetic backgrounds. The small number of mutant mice that survived for more than one month is exclusively on the Sv129xBalb/c background but its hemopoietic populations were not any different from the majority of homozygous animals analyzed.

Ikaros −/− Mutant Mice have a Rudimentary Thymus with no Definitive T cell Progenitors

Gross anatomical examination of the thoracic cavity in Ikaros −/− mutant mice at 2-3 weeks of age failed to identify a thymic gland. However, upon careful microscopic inspection, a rudimentary organ was observed. The thymic rudiment was often found in adipose tissue and sometimes was located at a higher position in the thoracic cavity than the thymus in normal, age matched animals. The location and the often non-fused bibbed appearance of this thymus resemble those of the early embryonic organ. This mutant thymus contained approximately 1×10⁵ cells in contrast to the 1-2×10⁸ cells regularly obtained from wild type littermates. This thymic rudiment was difficult to identify in one week old mutant mice but it was easier to detect after the third postnatal week. The density of nucleated cells in the mutant thymus was low when compared to the cellularity of the normal thymus. Eosinophils detected in the wild type thymus were also seen in the mutant organ especially around the portal arteries.

Thymic rudiments from Ikaros −/− littermates (two to four mice depending on litter availability) were pooled and analyzed by fluorescent antibody staining and flow cytometry. Forward and side scatter analysis of the Ikaros −/− thymocytes revealed a smaller size population compared to wild type controls. The cell composition of the thymus in Ikaros mutant mice (1×10⁵ cells recovered per thymus) and wild type littermates (2×10⁸ cells recovered per thymus) was determined. Cells were double-stained with: anti-CD4^{PE}/anti-CD8^{FITC}, anti-CD3^{PE}/anti-TCRαβ^{FITC}, anti-Thy 1.2^{PE}/anti-CD25^{FITC}, anti-CD4^{PE}/anti-HSA^{FITC}. Forward and side scatter analysis was performed on Ikaros −/− and wild type thymocytes to estimate the size and complexity of this population. Combinations of antibodies specific for Thy-1/CD25, CD4/CD8, CD3/TCRαβ, and CD4/HSA antigens were used to stain the Ikaros −/− and wild type thymocytes. These combinations of antigens demarcate the earliest and the later stages in T cell development (reviewed by Godfrey, D. I. and Zlotnik, A. (1993) Immunology Today; von Boehmer, 1993 #188; Weisman 1993). The wild type thymus contained the normal complement of mature and immature thymocytes. In sharp contrast, 95% of the mutant organs were devoid of single or double positive CD4 or CD8 cells and lacked cells that stained positively for CD3, TCRαβ, Thy-1 or CD25 (IL-2 receptor) (data is from two week old animals). The majority of these thymic cells stained positive with HSA known to be expressed on 95% of hemopoietic cells apart from early T and B cells. Interestingly, a small CD4^{1o}/HSA+ subpopulation was detected in some cases. The HSA+ cells detected in the Ikaros −/− thymus may belong to other hemopoietic lineages. Alternatively these cells may represent the earliest T cell progenitors, closely related or perhaps identical to the HSC, which lack expression of any definitive T cell markers. These putative T cell precursors may be arrested at the entry point into the T lymphocyte pathway.

Ikaros −/− Mutant Mice Lack Peripheral Lymphoid Centers

Inguinal, cervical, axillary and mesenteric lymph nodes were absent by both visual and microscopic examination. Lymph nodes were absent in all of the Ikaros mutant mice examined but were readily detected in all of the wild type littermates. Peyer's patches and lymphocyte follicles were also absent from the gastrointestinal tract of the Ikaros −/− mutant mice but were present in the wild type intestines and colon.

Dendritic Epidermal T Cells are Absent in Ikaros −/− Mice

Epidermal sheets from ear skin from Ikaros −/− and wild type mice were examined for γδ T cells and for Langerhan cells. Ammonium thiocyanate-separated epidermal sheets were stained for immunofluorescence microscopy with fluorescein (FITC) conjugated monoclonal antibodies specific for γδ T cell receptors (mAb GL3) or unconjugated monoclonal antibodies specific for Class II molecules followed by FITC conjugated goat anti-mouse antibody as described in Bigby, M. et al. ((1987) J. Invest. Dermatol. 89:495-499), and Juhlin, L. and Shelly, W. B. ((1977) Acta Dermatovener (Stockholm) 57:289-296)). Isotype control antibodies were used as negative controls for GL3 and M5/114. Positively stained dendritic cells were identified by epifluorescence microscopy. Ears from three mice of each type were examined. γδ T cells were absent from epidermal sheets from Ikaros −/− mutant mice but were readily detectable in epidermal sheets from wild type mice. Staining with the Class II antibody revealed the presence of dendritic epidermal Langerhan cells in both mutant and wild type epidermis.

Hemopoietic Populations in the Bone Marrow of Ikaros −/− Mice

Hemopoietic populations in the bone marrow of the Ikaros −/− mice were analyzed by flow cytometry using antibodies to lineage specific differentiation antigens. Cells from the bone marrow of Ikaros mutant mice (3-10×10⁷ cells per animal) and wild type littermates (4-10×10⁷ cells per animal) were analyzed with the following combinations of mAbs: CD3^{PE}/Thy1.2^{FITC},Thy1.2^{PE}/Sca-1^{FITC}, CD3^{PE}/TCRαβ^{ΦITC},CD45R^{PE}/IgM^{FITC}, CD45R^{PE}/CD43^{FITC}, Mac-1^{PE}/Gr-1^{FITC}, Ter 119^{PE}/CD61^{FITC}.

Ikaros −/− mice were analyzed and compared to age matched wild type controls. At least six groups of animals were studied on each mixed background (SV129xC57BL/6 and on SV129xBalb/c) and one on Sv129. Each group consisted of pooled organs from one to four littermates at 2 to 3 weeks of age. Older animals (1 month +) were examined individually. Red blood cells in the spleen and bone marrow were lysed by ammonium chloride. Single cell suspensions of thymus, spleen or bone marrow cells were prepared and washed twice in staining wash (PBS with 0.1% BSA), incubated for 20 minutes on ice with a 1: 20 dilution of normal rat serum and 1 μg mAb 2.4G2 (PharMingen, San Diego, Calif.) per 1×106 cells to block Fc receptors. Cells (1×10⁶) were incubated with PE conjugated mAb and FITC conjugated mAb for 40 minutes. 2×10⁴ thymocytes were stained with appropriate combinations of PE and FITC conjugated mAbs since few cells were recovered from mutant thymus. Cells were then washed 3 times and one- and two-color flow cytometric analyses were performed on a FACScan (Becton-Dickinson, San Jose, Calif.). Gating for viable cells was performed using propidium iodide exclusion and SSC and FSC as described (Yokoyama, W. M. et al. (1993) “Flow Cytometry Analysis Using the Becton Dickinson FACScan. In Current Protocols in Immunology, Coligan, J. E. et al., eds. (Greene Publishing Associates, New York) 5.4.1-5.4.14. Isotype matched control antibodies were used as negative controls. Ten-thousand cells were analyzed for each sample.

The first stages of B cell development take place in the late mid-gestation liver and spleen in the embryo, and in the bone marrow in the adult (Li, Y. -S. et al. (1993) J. Exp. Med. 178:951-960). These stages are demarcated by the sequential activation of cell surface antigens. Combinations and levels of expression of these stage specific markers are used to define the pro-B to pre-B stage (CD45R+/CD43+) and the pre-B to the B cell transitions (CD45R+/sIgM+) (Ehlich, A. et al. (1993) Cell 72:695-704; Hardy, R. R. et al. (1991) J. Exp. Med. 173:1213-1225; Li, Y. -S. et al. (1993) J. Exp. Med. 178:951-960; Rolink, A. and Melchers, R. (1991) Cell 66:1081-1094). In wild type bone marrow, the CD45R+ population contains B lymphocytes at various stages of their maturation. The small CD45R+/sIgM+ population consists of mature B cells while the even smaller population of CD45R^{1o}/CD43R^{1°} cells contain immature lymphocytes at the pro-B cell stage (data shown is from a group of two week old animals).

The rest of the CD45R+ population consists of pre-B cells with rearranged heavy but not light chains as well as other hemopoietic cells. The CD45R+ population was greatly reduced and in many cases absent in the Ikaros mutant mice. The CD45R+ cells detected were low expressors and were negative for either CD43 or IgM. These cells may derive from an even earlier stage in B cell development than the one defined by the CD45R+/CD43+ combination. Alternatively they may belong to the CD5 lineage of B cells or to another hemopoietic lineage (Hardy, R. R. et al. (1986) J. Exp. Med. 173:1213-1225 and Herzenberg, et al., 1986).

T cell progenitors originate in the bone marrow in the adult and in the fetal liver in the embryo but the first definitive steps in T cell differentiation occur after their migration to the thymus. Given the lack of substantial numbers of defined T cell progenitors in the thymic rudiment of the Ikaros −/− mice, we examined their presence in the bone marrow. In most Ikaros −/− mice, a small population of Thy-11° positive cells was present. These cells were not positive for CD3, Sca-1 or CD4 antigens which are expressed on early but definitive T cell precursors. This population of Thy- 1 lo cells in the bone marrow of Ikaros −/− mice may contain the earliest lymphocyte progenitors including T and B cell precursors that are arrested in development and therefore unable to home to the thymus or proceed to the next stages differentiation.

The majority of nucleated cells in the bone marrow of Ikaros −/− mice were of the erythroid lineage. The proportion of erythrocyte precursors was larger in the Ikaros mutant mice than in wild type controls (53 vs. 31 %). At two weeks of age, a similar number of bone marrow cells were positive for the myeloid lineage marker Mac-1 in the Ikaros −/− mice and in their wild type littermates (19 vs. 23% Mac-1+) which suggested that their myeloid compartment was also intact. However, in most cases the Mac-1+/Gr-1+ subpopulation that correlates with polymorphonuclear cells of a more mature granulocytic phenotype was not present among these Mac-1+ cells in most of the Ikaros mutant mice (Hestdal et al., 1991; Fleming et al., 1993, Lagasse and Weissman, 1993). Nevertheless, special stains and histological examination on blood smears and infected tissue has identified numerous circulating and infiltrating cells with mature polymorphonuclear and granulocytic morphology.

The Spleens of the Ikaros −/− Mutant Mice are Enlarged and Heavily Populated with Cells of Erythroid and Myeloid Origin

Tissues harvested from euthanized wild type and Ikaros mutant mice were fixed in 4% buffered formalin for 1-2 days. They were then processed and embedded in paraffin. Sections were cut at 5 micron thickness, mounted and stained with hematoxylin and eosin or with modified gram stains. Light microscopy was performed at 20-600×magnification on an Olympus BMax-50 microscope. The spleens from the Ikaros −/− mice were enlarged compared to the wild type littermates. This size difference varied from one and a half to three times the size of the wild type spleen. The enlarged size of the Ikaros −/− spleens was in contrast to the absence of peripheral lymphatic centers and to the diminished size of the thymus detected in these mutant animals. The red and white pulp architecture of the wild type spleen was absent in the mutant organ. The white areas detected in mutant spleen were heavily populated with cells of myeloid morphology (m) and were surrounded by red areas populated by erythrocyte (e) precursors. A large number of megakaryocytes were also detected throughout these splenic sections

The splenic populations in the Ikaros −/− mice were examined by flow cytometry to delineate the relative representation of the hemopoietic lineages. Single CD4+ and CD8+ cells which together comprise approximately 40% of spleen cells in normal mice were absent in all of the Ikaros −/− mice examined. αβ and γδ T cell receptor expressing cells were similarly absent from the Ikaros −/− spleens. However, a small but distinct population of Thy-1^{1°} cells which were CD3- and Sca-1- was present as in the bone marrow.

The CD45R+/IgM+ population that represents the transition from the pre-B to the B cell stage in normal spleen was absent from this mutant organ. The CD45R+/CD43+ population that represent the pro-B to pre-B cell transition in the wild type bone marrow were not detected in either wild type or Ikaros −/− spleens.

The majority of the spleen cells in the Ikaros −/− mice were erythrocyte progenitors (TER119+). This population which ranged from 70% at 1-2 weeks of age to 25% in older mutant mice, never exceeded 20% in the spleen of wild type controls. Myeloid cells comprised the second predominant population in the spleen of Ikaros mutant mice and ranged from 9% in young animals to 60% in older mice. In the spleen of wild type mice, myeloid cells never exceeded 5%. In the Ikaros mutant spleen, the erythroid and myeloid lineages together accounted for the majority of the cells (80-100%). In contrast, in the wild type spleen these two lineages represent less than 20% of the total cell population which is accounted for by mature T and B cells.

The presence of myeloid progenitors in the spleen of Ikaros mutant mice was tested in a soft agar clonogenic assay. A large number of mixed macrophage and granulocyte (GM) colonies were established when spleen cells from two-week old mutant mice were grown on soft agar in the presence of GM-CSF (Table 1). Spleen cells from wild type littermates gave only a small number of mixed GM colonies. Similar numbers of mixed GM colonies were derived from cells from the spleen and bone marrow of mutant mice whereas in wild type animals' bone marrow and spleen derived GM colonies differed approximately by ten fold (Table 1).

TABLE 1
G/M progenitors in the spleen and
bone marrow of Ikaros −/− mice

| | Experiment 1 | | | | Experiment 2 | | |
|---|---|---|---|---|---|---|---|
| Spleen | | Bone | marrow | Spleen | | Bone | marrow |
| +/+ | −/− | +/+ | −/− | +/+ | −/− | +/+ | −/− |
| 3 | 38 | 38 | 55 | 8 | 85 | 58 | 100 |

Natural Killer Cell Activity was Absent from the Spleens of Ikaros −/− Mice

NK cells do not appear to be present in the spleen of the Ikaros −/− mice (as detected by flow cytometry). A small population of these cells was present in wild type spleens (2-5% determined on the SV129xC57BL/6 background). Given the relatively small numbers of splenic NK cells, a functional assay was used to conclusively address their existence. Serial dilutions of spleen cells from Ikaros mutant and wild type animals were grown in the presence of 500 units/ml of IL-2 for 48 hours. These conditions are known to generate activated NK cells which can readily lyse their targets (Garni-Wagner, B. A. et al. (1990) J. Immunol. 144:796-803). After two days in culture, spleen cells from wild type control mice effectively lysed chromium labeled NK cell targets (Yac-1) over a wide range of effector to target cell ratios (Table 2). However, spleen cells from the Ikaros −/− mice were unable to lyse NK targets even at the highest effector to target cell ratio (60:1)(Table 2).

TABLE 2
NATURAL KILLER CELL ACTIVITY^{a}

| | Percent Lysis^{b} | | | |
|---|---|---|---|---|
| | Experiment 1 | | Experiment 2 | |
| Effector to | | | | |
| Target Ratio | +/+ | −/− | +/+ | −/− |
| 60:1 | 59 | 1 | ND | ND |
| 30:1 | 48 | 2 | 75 | 4 |
| 15:1 | 43 | 4 | 57 | 10 |
| 7.5:1 | 16 | 4 | 29 | 2 |

a. Spleen cells from wild type (+/+) or Ikaros deletion (−/−) mice were cultured in complete RPMI containing 500 units/ml recombinant IL-2 for 72 hours and were then cultured in triplicate with 3000 CR⁵¹ labeled Yac-1 cells in indicated ratios in a standard 4 hour chromium release assay.

$$b . Percent lysis = \frac{[ CPM - Spontaneously released CPM ] \times 100}{[ Total lysis CPM - Spontaneously released CPM ]}$$

Analysis of Ikaros Mutant mRNAs and Proteins

The production of Ikaros mRNAs in the spleen of Ikaros mutant mice was investigated using a reverse transcription PCR amplification assay (RT-PCR). Georgopoulos, K. et al. (1992) Science 258:808. Primers derived from the Ikaros exons within and outside the targeted deletion were used to amplify cDNAs prepared from Ikaros −/− spleen. These primers, Ex2F/Ex7R, Ex2F/Ex6R, Ex3F/Ex7R, Ex4F/Ex7R, allow the determination of exon usage by the Ikaros transcripts.

| | |
|---|---|
| Ex2F: CAC TAC CTC TGG AGC ACA GCA | (SEQ ID NO:20); |
| GAA | |
| Ex3F: AGT AAT GTT AAA GTA GAG ACT | (SEQ ID NO:17); |
| CAG | |
| Ex4F: GGT GAA CGG CCT TTC CAG TGC | (SEQ ID NO:21); |
| Ex6R: TCT GAG GCA TAG AGC TCT TAC | (SEQ ID NO:22); |
| Ex7R: CAT AGG GCA TGT CTG ACA GGC | (SEQ ID NO:23). |
| ACT | |

zinc finger modules -1, -2 and -3 of Ikaros encoded by the deleted exons 3 and 4 are responsible for the specific DNA contacts of the Ikaros proteins (Molnar et al., 1994a). cDNAs from wild type (+/+) thymus (T) or wild type and mutant (−/−) spleens (S) were PCR amplified with sets of primers that delineate their exon composition (primer sites are shown as filled boxes). These sets of primers amplified from wild type thymus and spleen predominantly products of the Ik-1 and Ik-2 transcripts as previously described (Molnar et al., 1994a). The major amplification product from the Ikaros mutant spleen cDNAs did not contain exon 3 and exon 4 but consisted of exons 1-2-5-6-7. The presence of Ikaros related DNA binding complexes were examined in nuclear extracts prepared from wild type thymus and from wild type and mutant spleen. Four sequence specific DNA binding complexes (arrows) were established by DNA competition assays. The presence of Ikaros proteins in these nuclear complexes was established by Ikaros specific and non-specific antibodies. These complexes are absent altogether from mutant spleen nuclear extracts which however support the formation of DNA binding complexes over an AP-1 site.

Analysis of these amplified products revealed the production of Ikaros mRNAs. These Ikaros mRNAs lack exons 3 and 4 and the major species corresponds in size to a transcript comprised of exons 1-2-5-6-7. Proteins encoded by these Ikaros mRNAs lack the DNA binding zinc fingers -1, -2 and -3 encoded by exons 3 and 4 (Molnar, et al., 1994).

The absence of Ikaros related DNA binding complexes in the hemopoietic populations of Ikaros mutant mice was confirmed in a gel retardation assay. Nuclear extracts were prepared and gel retardation assays were carried out as previously described. Georgopoulos, K. et al. (1992) Science 258:808. 2 μgs of nuclear extract were incubated with end labeled oligonucleotides containing either a high affinity Ikaros (IKBS4) or an AP-1 binding site.

| | | |
|---|---|---|
| IK- | TCAGCTTTTGGGAATGTATTCCCTGTCA | (SEQ ID NO:24); |
| BS4: | | |
| IK- | TCAGCTTTTGAGAATACCCTGTCA | (SEQ ID NO:25); |
| BS5: | | |
| AP1: | GGC ATG ACT CAG AGC GA | (SEQ ID NO:26). |

Nuclear extracts prepared from two week old wild type thymus and wild type and mutant spleens were tested for binding to a high affinity recognition sequence for the Ikaros proteins (Molnar, et al., 1994). Four DNA binding complexes with distinct mobilities were detected when nuclear proteins from wild type thymus and spleen were used. However, none of these four DNA binding complexes was formed when splenic nuclear extracts made from Ikaros mutant mice were used. Nevertheless, these nuclear extracts supported the formation of DNA binding complexes over an API binding site. Competitor DNA with a high affinity recognition site for the Ikaros proteins abrogated binding of all four complexes while DNA with a mutation in the binding consensus for the Ikaros proteins had no effect (Molnar, et al., 1994). Pretreatment of the thymic nuclear extract with Ikaros antibodies also abrogated all four of these DNA binding complexes whereas an unrelated antibody showed no effect. These data indicate that nuclear complexes which contain Ikaros proteins are present in cell populations in the thymus and spleen of wild type animals but are absent in the spleen cells of the homozygous mutants.

Opportunistic Infections and Death in Ikaros −/− Mice

Deaths of Ikai os −/− mice occurred as early as the end of their first postnatal week. The mortality rate increased during the second and the third weeks of life. Approximately 95% of the mice died within 4 weeks. Gross and histopathological examination of the mouse gastrointestinal tract, liver, lung and blood was performed to evaluate the cause of their death.

Examination of the intestines did not reveal major histopathological abnormalities, however, Ikaros −/− mice consistently had numerous and diverse bacterial microorganisms in their intestinal tract. Large numbers of gram negative and positive rods and cocci were detected on tissue gram stains of intestinal sections from the mutant mice. Although a small number of bacteria were observed in wild type intestinal epithelia, their number and diversity did not compare to that detected in mutant mice. Cultures from gastrointestinal epithelia from Ikaros −/− mice identified a number of proliferating microorganisms. Interestingly, anaerobic endospore-forming bacteria of the Oscillospira caryophanon group were found at a highly prolific state in the intestines of the Ikaros mutant mice while they were not detected in wild type controls.

The liver in almost all animals examined contained focal infarcts that appeared as pale or white nodules. In extreme cases, half of the liver had undergone necrosis. Necrotic areas and accumulation of large numbers of monocytes, macrophages and eosinophils were present on hematoxylin and eosin stained liver sections. Hematoxylin and eosin staining of lung tissue from one-month old mutant animal revealed the destruction of normal tissue structure, bacterial abscessae and myeloid infiltration. This staining exhibited necrotic areas and bacterial growth mainly at the subcapsillary region and extensive infiltration with myelocytes and eosinophils. Cultures from the liver grew pasturella pneumonotropica and enterobacteria species, microorganisms which comprise part of the microbial flora in the oral and gastrointestinal cavities of normal mice. Cultures from wild type liver had no growth. In a Wright stain of blood smears from a one-month old Ikaros mutant mouse, basophils were the prevalent leukocyte population detected and were found concentrated over clusters of bacteria. The bacteria identified on Wright stained blood smears indicated high-grade septicemia (Fife, A. et al. (1994) J. Clin. Pathol. 47:82-84). Blood clots were cultured and frequently contained multiple strains of microorganisms.

Ikaros and Hematopoietic Development

The analysis of mice with a mutation in the Ikaros gene provides convincing evidence that the Ikaros gene plays a pivotal role in lymphocyte specification. An intact Ikaros gene is essential for the development of T and B lymphocytes and NK cells. The Ikaros gene is not essential for the production of totipotential hemopoietic stem cells, erythrocytes, myelocytes, monocytes, dendritic cells, megakaryocytes and platelets.

As shown above, a mutation in the Ikaros gene that abolishes the DNA binding domain in at least four of its protein products has profound effects on T lymphocyte development. T cell differentiation is arrested at a very early stage. Ikaros −/− mutant mice have a rudimentary thymus which contains 1×10⁵ cells, 2000 times less than the wild type organ. These cells are HSA+ with a small subpopulation approximately 10% expressing low levels of HSA and CD4. No other definitive early T cell marker, e.g., Thy-1, Sca-1, CD25, CD3 was expressed on these cells. The majority of these HSA+ cells in the Ikaros −/− thymus may belong to other hemopoietic lineages. Alternatively, they may contain small non cycling T cell progenitors arrested at a very early stage of intrathymic differentiation. The Thy-1+CD3-SCA-1⁻ cells detected in the bone marrow and spleen of the Ikaros mutant mice may also contain arrested T cell progenitors which may lack expression of the appropriate surface receptors that enable them to home to the thymus.

Lymphocyte progenitors that give rise mainly to the γδ T lineage populate the thymus from day 14 through day 17 of fetal development (Havran, W. L. and Allison, J. P. (1988) Nature 344:68-70; Ikuta, K. et al. (1992) Annu. Rev. Immunol. 10:759-783; Raulet, D. H. et al. (1991) Immunol. Rev. 120:185-204). Mature γδ T cells produced during this time populate the skin and vaginal epithelium and provide the life long supply of dendritic epidermal T cells (Asnamow, D. M. et al. (1988) Cell need volume: 837-847; Havran and Allison, 1990; Havran, W. L. et al. (1989) Proc. Natl. Acad. Sci. USA 86:4185-4189). The absence of γδ T cells in Ikaros −/− mice implies that this stage in T cell ontogeny is never reached in these animals.

The Ikaros mutation has profound effects on the development of a third lineage of T cells, that of NK cells. Since these cytotoxic cells share differentiation antigens with T cells it has been proposed that they may be derived from a common progenitor (Rodewald, H. et al. (1992) Cell 139-150). Differentiation experiments with committed T cell progenitors have failed to generate the expected NK cell activity (Garni-Wagner, B. A. et al. (1990) J. Immunol. 144:796-803). Nevertheless, a common bipotential progenitor may exist which may not have a definitive T cell phenotype definable by early T cell differentiation antigens e.g. HSA, pgpl, CD4 and CD25. This progenitor pool may be part of the cell population detected in the Ikaros mutant thymus.

Many immunodeficient animals which do not produce mature lymphocytes appear to live well under relatively germ free conditions. This fact has been partly a attributed to the high numbers of circulating NK cells in these animals (Mombaerts, P. et al. (1992) Cell 68:869-877; Shinkai, Y. et al. (1992) Cell 68:855-867; Spanopoulou, E. et al. (1994) Genes Dev.). In contrast, Ikaros mutant mice fail to thrive even in relatively germ free conditions. A majority of these animals die soon after birth. Septicemia is the major cause of death in these animals. The rapid development of bacterial infections in Ikaros −/− animals may be due to the lack of NK cells in addition to lack of T and B lymphocytes.

No mature B cells or any of their well-defined progenitors were found in the bone marrow or the spleen of the Ikaros mutant mice. A small population of CD45R^{1°} cells was detected which did not express CD43 or IgM, surface markers characteristic of the pro-B and pre-B cell transition. This total lack of T and B cell progenitors is unprecedented among naturally occurring and genetically engineered immunodeficient mice (Karasuyama, et al.; Mombaerts, P. et al. (1992) Cell 68:869-877;Shinkai, Y. et al. (1992) Cell 68:855-867) suggesting that Ikaros mutant mice may be arrested at the hemopoietic stem cell level before lymphocyte specification. The described functional disruption of the Ikaros gene may affect the development of a progenitor stem cell that gives rise to T, B and the NK cell lineages. However, the Ikaros gene products may control the development of three distinct progenitors each responsible for giving rise to a distinct lymphocyte lineage with each lineage arrested at the very first steps of its ontogeny.

Profound effects from this Ikaros mutation were also seen on the population dynamics of the erythroid and myeloid lineages. The relative proportion of erythroid and myeloid progenitors were increased in the bone marrow and especially in the spleen of Ikaros mutant mice compared to their wild type littermates. However, the absolute number of hemopoietic cells was lower in the bone marrow but higher in the spleen of mutant mice. These observations were in contrast to other immunodeficient mice where lack of mature T and B lymphocytes dramatically decreased the number of hemopoietic cells in the spleen but had smaller effects on bone marrow populations. These results may have several explanations.

One possibility is that one of the functions of the Ikaros gene products, potentially expressed in the pluripotential hemopoietic stem cell (HSC), is to signal its differentiation into the lymphocyte lineage. FIG. 7 shows an Ikaros view of the hemopoietic system; expression and putative roles in differentiation. Ikaros expression at the various stages of hemopoietic development is an approximation (Georgopoulos, K. et al. (1992) Science 258:808). Expression data was derived from Northern and PCR analysis of primary cells and cell lines and by in situ hybridization of fetal hemopoietic centers. Relative levels of expression (+) or lack of (−) are shown at various stages in development. Potential inductive signals for lymphocyte commitment and differentiation provided by the Ikaros gene are shown as arrows. Interrupted lines indicate putative Ikaros related negative signals for differentiation in the erythroid and myeloid lineages. Transitions in the lymphocyte pathway during which development is probably aborted in Ikaros −/− mice are drawn as Xs on the pathway. Dashed lines indicate unsettled transitions in lymphocyte differentiation, e.g. the existence of a common committed progenitor for the T and B lineages or their independent derivation from the pluripotent hemopoietic stem cell is a controversial issue (Ikuta, K. et al. (1992) Annu. Rev. Immunol. 10:759-783). In addition the origin of the T and NK lineages from a common committed T cell progenitor remains under debate (Hackett, J. J. et al. (1986) Proc. Natl. Acad. Sci. USA 83:3427-3431; Hackett, J. J. et al. (1986) J. Immunol. 136:3124-3131.; Rodewald, H. et al. (1992) Cell 139:150). Differentiation antigens representative of the various stages of hemopoietic and lymphocyte development (also used in the analysis of the Ikaros −/− mice) are shown. In the absence of these lymphocyte specific differentiation signals provided by the Ikaros gene products, the HSC is diverted by default into one of the other hemopoietic pathways.

The differentiation of HSC may be tightly regulated by Ikaros gene products which may provide both positive signals for lymphocyte differentiation and negative signals to prevent or attenuate entry into the other hemopoietic pathways (FIG. 7). Finally, the body may sense the lack of lymphocytes and may attempt to correct this defect by increasing hemopoiesis. However, since the lymphocyte pathway is blocked, stem cells produced will passively or actively generate more progenitors for the other non- lymphocyte hemopoietic lineages. This may explain in part the abundance of erythroid, myeloid and megakaryocyte progenitors encountered in Ikaros −/− mice. The increased levels of myelopoiesis relative to erythropoiesis detected in older mutant animals may be caused by infections and septicemia that develop in these animals.

Ikaros gene products expressed during the earliest stages of fetal hemopoiesis (before the development of the lymphopoietic system) may influence the hemopoietic system in other ways than directing HCSs toward lymphocyte lineage commitment. HCSs have distinct migration pathways in the embryo and in the adult (Ikuta, K. et al. (1992) Annu. Rev. Immunol. 10:759-783). The migration of HCSs from one organ to another during embryonic development and the switch from embryonic to adult hemopoiesis that takes place at the HSC level may be in part controlled by the Ikaros gene (FIG. 7). The hypocellular bone marrow in the Ikaros mutant mice may result from a failure of HCS to migrate to the bone marrow and the high degree of extramedullary erythropoiesis and myelopoiesis detected in the spleen of these animals may result from dysregulated transition from embryonic to adult hemopoiesis. Alternatively lack of thymocyte progenitors in the Ikaros mutant mice may hinder the homing of the HSC into bone marrow cavities. The spleen may become the primary site of extramedullary hemopoiesis in Ikaros mutant mice because the hemopoietic compartment in the bone marrow is severely deficient.

The Ikaros gene plays an essential role for lymphocyte specification in the mouse hemopoietic system. Absence of functional Ikaros proteins leads to a total blockade in the development of T cells, B cells and NK cells. Ikaros mutant mice will provide an experimental system for addressing the molecular components which exist downstream of the Ikaros gene and whose expression is detrimental for lymphocyte specification and development.

An Ikaros Transgenic Mouse with a Deletion at Exon 7 of the Ikaros Gene

The Ikaros gene is believed to be a necessary factor for the generation and maintenance of early hemopoietic progenitors since it is expressed during embryonic hemopoiesis prior to lymphocyte ontogeny (fetal liver day 10). A mutation at the Ikaros locus which brings about a total loss of function at the level of its transcription activators and suppressors can lead to an embryonic lethal due to an impairment in the production of embryonic blood.

A recombination vector targeting a deletion to the C-terminal part of the Ikaros proteins was made and used to generate transgenic animals heterozygous and homozygous for a deletion in exon 7. This mutation is expected to generate proteins that appear only partially active in transcription.

Transcripts from this mutated locus lack exon 7. The encoded proteins, are expected to bind homologous or heterologous nuclear factors during lymphocyte development. This mutation is expected to interfere with the role of the Ikaros proteins in gene regulation but is not expected to totally abrogate their function in lymphocyte transcription.

Truncated Ikaros isoforms lacking the C-terminal domain encoded by exon 7 and shared by all of these proteins can bind DNA with the same specificity as their full-length counterparts (as determined by gel retardation assays). However the ability of these truncated proteins to activate transcription appears to be significantly lower than that of their full-length counterparts as determined in transient expression assays and experiments using Ikaros-lex-A hybrid proteins. Acidic motifs present in this C-terminal portion may serve as potential transcription activation domains and may be responsible for this effect. Deletion of an activation domain located in the deleted C-terminal region may be responsible for the decrease on their ability to activate transcription. The deleted C-terminal region contains in addition to the activation a dimerization domain for the Ikaros proteins established in the yeast two-hybrid system.

Replacement of 700 bp of exon 7 by the neomycin gene gave rise to translation products which stop short of the shared C-terminal domain. These proteins are expected to bind DNA since they have a high affinity DNA binding domain at their N-terminus. However they should be compromised in their ability to activate transcription since part of their activation domain resides in their C-terminus. In lymphocytes heterozygous for this mutation, these mutant proteins may compete with their wild type counterparts for binding sites thus interfering with their function and with normal lymphocyte differentiation. Hematopoietic stem cells homozygous for this mutation may exhibit partial to total loss of Ikaros function depending on the ability of these truncated proteins to support transcription in vivo. The hematopoietic phenotype manifested by these cells can vary from an early to a late lymphocyte arrest or to aberrant events in T cell homeostasis.

The hemopoietic Populations of Mice Homozygous for the C-terminal Ikaros Mutation

Two independent embryonic stem cell lines with legitimate homologous recombination events were used to generate mice with germ line transmission of this mutation. Mice homozygous for this Ikaros mutation are born with the expected Mendelian frequency and are indistinguishable from wild type littermates unless they are infected by opportunistic microorganisms. However the level of infections is not as extensive as with the N-terminal mutant homozygous mice and many animals survive for extended periods under sterile conditions. Male mutant homozygotes have successfully been bred with female heterozygous mutants.

Analysis of the hemopoietic system of a number of homozygous animals was performed. In contrast to the microscopically detectable thymic rudiment in the line of homozygous animals described above (the exon 3/4 deletion), this line of C-terminal homozygous mutants have a normal sized thymus. However, the ratio of CD4⁺, CD8⁺ and CD4⁺/CD8⁺ populations differed from those in wild type controls. The CD4⁺/CD8⁺ population was decreased in both healthy but mostly in the sick animals while the CD4⁺ population was increased. Increased numbers of mature CD4⁺ T cells were also detected in the spleen of healthy animals, while the CD8⁺ population was similar in numbers to wild type littermates. However in many sick homozygous mice, these mature CD4⁺ and CD8⁺ populations but predominantly the CD4⁺/CD8⁺ cells were greatly diminished.

In contrast to the presence of T lymphocytes from the early to the late stages of their development, B cells and their earliest identifiable progenitors were absent from all the hemopoietic centers analyzed in the Ikaros C-terminal −/− mutant mice.

The myeloid and erythroid lineages in these hemopoietic organs were intact and in a few cases elevated as in the N-terminal Ikaros homozygous mice. No peripheral lymphatic centers, i.e. inguinal, cervical, axillary and mesenteric lymph nodes as well as Peyer's patches and lymphocyte follicles in the gastrointestinal tract were found in these Ikaros −/− mutant mice.

An Ikaros Transgenic Mouse with two Ikaros Mutations (One Ikaros Allele with a Mutation that Deletes the C-terminal Portion of the Protein, and the Other Ikaros Allele with a Deletion in its DNA Binding Domain)

Mice homozygous for a germ line deletion of exons encoding the DNA binding domain of the Ikaros proteins lack T, B and NK lymphocytes and their progenitors. Analysis of the hemolymphopoietic system of mice homozygous for a germ line deletion of the C-terminal part of the Ikaros proteins has begun. In addition, mice heterozygous for the C-terminal and DNA binding mutations have been bred with one another to determine whether the two mutations can functionally complement each other with intermediate effects or defects in the development of the lymphopoietic system.

Transgenic Mice Which Overexpress Ikaros Isoforms

Overexpression of Ikaros isoforms (Ik-1, -2, -4, -5) can be obtained by using the pMu expression cassette (to drive expression in the B lineage, 4 transgenic lines) or by using the CD2 mini gene (to drive expression in the T lineage, 4 transgenic lines).

Ikaros overexpression vectors have been generated using the immunoglobulin promoter enhancer regulatory sequences driving Ikaros isoform expression in the hemopoietic/lymphopoietic system. These vectors were generated in order to determine whether expression of Ikaros at the wrong times during development affects the developmental outcome of the B or T cell pathways and to reconstitute the genetic background of the Ikaros mutant mice and functionally dissect the Ikaros proteins.

Overexpression of Ik-1 in the myeloid lineage can be obtained by using the Mac-1 (CD11b) expression cassette. The expression cassettes are excised from the pGEM backbone and introduced into mouse male pronuclei where they integrate into the pronuclei chromosomes. The male pronuclei are then used to generate transgenic mice as described above.

Analysis of the 5′ Ends of Ikaros mRNAs Points to the Existence of Two Promoters

The Ikaros gene has been determined to span approximately 120 kb of DNA and is comprised of seven translated and two 5′ untranslated exons (FIG. 8A). Ikaros was cloned and mapped as follows. Two phage clones with insert sizes of 15 and 19 kb respectively which cover exons 3 through 7 were obtained by screening a λ DASHII library. A PI phage clone was obtained (Genome Systems, Inc. St Louis, Mo.) through hybridization to a 350 bp PCR fragment from a region encompassing the 5′ end Exon of 3. The genomic sequences contained within the PI clone spanned from about 35 kb upstream of exon 1 to about 5 kb downstream of exon 3. The two phage clones contained the 3′ of the locus from exon 3 to 10 kb downstream of exon 7. PI DNA was recovered using standard plasmid isolation protocols and PI Manual by Genome Systems, Inc. St Louis, Mo. Fragments resulting from an EcoRI and/or BamHI digest were subcloned into either Bluescript II SK or Bluescript II KS (both Stratagene). The subcloned fragments were mapped using Southern Blots of EcoRI, BamHI, Kpnl, EcoRV single double digests of PI DNA from clone 2528. These blots were hybridized to regions of Ikaros cDNAs and cloned PI fragments. A map of the locus was drawn corresponding to the information compiled from these autoradiographs. The phage clones were mapped and subcloned in similar fashion. All restriction endonucleases were obtained from New England Biolabs.

Characteristic of the locus is a 41 kb intron located between the translated exons 2 and 3 which contains three out of the eight clusters of tissue specific DNaseI HSS described below. To map the transcriptional start sites in the Ikaros gene, the genomic sequence was analyzed directly upstream of the first translated exon. A splice-acceptor sequence was identified which suggested that the Ikaros promoter region lies further upstream possibly at the 5′ end of an untranslated exon. To map the location of such a putative promoter, the 5′ end of Ikaros mRNAs were analyzed by 5′ RACE (Rapid amplification of cDNA ends) and by primer extension using primers from exons 1 and 2 (FIG. 8B).

The primer extension protocol used is according to Ausubel et al. (1999) Cell Immunol. 193(1):99-107 (Primer Extension) with a few modifications. Briefly, total RNA was prepared from Thymus, Spleen and Liver tissue using the guanidinium method (Ausubel et al. (1999)) (Single-Step RNA Isolation from Cultured Cells or Tissues). Subsequently poly (A)⁺ RNA was isolated using the Oligotex procedure (Qiagen). The protocol is described in “Oligotex mRNA Handbook” Qiagen Inc. 1995. 1×10⁵ cpm of a kinased and gel purified oligo was precipitated with 7.5 ug poly(A)⁺, 20 μg glycogen, 0.3 M NaAc, pH 5.5 in 100 μl final volume through the addition of 270 μl of 100% ethanol. The pellet was washed with 100%ethanol and then air-dried. Subsequently, the pellet was resuspended in 30 μl 1× hybridization (150 mM KCl; 10 mM Tris-Cl, pH 8.3; 1 mM EDTA), incubated at 85° C. for 10 minutes and then transferred to a 30° C. waterbath for 12 hours. The hybridization solution was brought to a final volume of 200 μl with H20, then precipitated with 400 μl ethanol. The pellet was washed with 70% ethanol, air dried and resuspended in 18.4 μl 1× reverse transcription buffer (4 μl of 5× first strand buffer (GibcoBRL); 0.4 μl of 0.1 M DTT; 8 μl of 2.5 mM dNTPs (Boehringer); 6 μl of H₂O), 0.6 μl of PRIME RNase inhibitor (5′AΣ3′, Inc.) and 1 μl of reverse transcriptase (Superscript II, Rnase H Reverse Transcriptase, GibcoBRL) was added. This was incubated in a 42° waterbath for 2 hours. Subsequently, 1 μl of Ribonuclease H (GibcoBRL) was added and incubated for 30 minutes at 37° C. The solution was then Phenol/Cloroform/isoamylalcohol (50/49/1) extracted after the addition of 150 μl STE. Then the DNA was precipitated with 500 μl ethanol. After a washing (70% ethanol) and air drying, the pellet was resuspended in 10 μl loading buffer (80%(vol/vol) formamide; 1 mM EDTA pH 8.0; 0.1% Bromophenol Blue; 0.1% Xylene Cyanol). Before loading on a 6% acrylamide/bisacrylamide (29:1), 7 M urea gel the samples were incubated at 80° C. for 5 minutes. As a size reference a sequencing reaction was run next to the sample. FIG. 9B shows the autoradiography of a characteristic primer extension analysis done with a P32 labeled primer that lies in exon 2 (C29). C29 primer sequence: cct tca tct gga gtg tca ctg act g (SEQ ID NO:______).

For RACE analysis, primer C29 was hybridized to 7.5 ug poly (A)+ selected RNA and reverse transcribed as described in ‘5′ RACE System for Rapid Amplification of cDNA Ends’ kit from GibcoBRL (Cat. No. 18374-025). The resulting cDNA was 3′ tailed with dCTP using the terminal deoxynucleotide transferase (GiccoBRL). The product was then PCR amplified with the nested primer C50 and a poly G /adaptor primer (GibcoBRL). As a negative control for the PCR reaction, the product of the PCR reaction was used with the exception that it lacked the 3′ poly C tail (no TdT reaction). C50 primer sequence: ctg aaa ctt ggg aca tgt ctt g (SEQ ID NO:______).

Primer extension with a primer deduced from exon 2 (C29) identified a major product of 327 bp which was highly enriched in mRNA from the thymus, was detectable in the spleen but not in the liver, thus recapitulating Ikaros expression or lack of it in these tissues. The size of the primer extension product shifted accordingly when a primer from exon 1 was used (C50-data not shown). Some larger and smaller but less abundant primer extension products (XX-319-280 bp) were also seen in the thymus and spleen but not in the liver. The 5′ ends of Ikaros mRNAs were cloned from the thymus by 5′ RACE. Sequencing of the RACE products revealed two types of untranslated sequence, designated as R10 and R19, that were independently spliced to exon 1. R10 was the longest and most abundant of the two RACE products and correlated with the largest and most abundant primer extension product. Two exons encoding the R10 and R19 sequences were located 10 and 15 kb, respectively, upstream of exon 1 (FIG. 8A). Sequence analysis of these regions revealed absence of a splice acceptor site and the presence of GC rich sequences frequently found in hemo-lymphoid-specific promoters. The non-canonical (non TATA box) nature of these promoters may account for a somewhat variable transcription start site that can give rise to the multiple primer extension products detected.

Taken together these studies show the possible utilization of two promoters in the Ikaros locus located upstream of two untranslated exons, R10 and R19, that splice independently to the first translated exon. These putative promoters are associated with two distinct clusters of lymphoid-specific DNaseI HSS (FIG. 9A, cluster β and γ) which are possibly active in distinct cell types.

The Ikaros Locus Contains Eight Distinct Regions of Accessible Chromatin in Lymphocytes

To identify the regulatory regions responsible for Ikaros expression, lymphoid specific DNaseI HSS were searched for. These are indicative of altered chromatin structure that results from the action of tissue-specific regulatory factors. DNaseI hypersensitivity assays were performed as follows. Nuclei were isolated from splenic, thymic and liver single cell suspensions and were treated with 0-20 units of DNase I (Sigma), as previously described Wu, 1989. DNA was isolated and digested with the appropriate restriction enzyme indicated (EcoRI, BamHI; EcoRI-BamHI, all New England Biolabs), run on an 1% agarose gel, and transferred on Hybond %o N+ membrane (Version 2.0, Amersham Life Science). The Southern transfers were probed with genomic fragments indicated in FIG. 8A. Probes were labeled by the oligonucleotide random priming method (NEBlot Kit, New England Biolabs). The restriction enzymes used to identify the various DNase I HS regions in the genomic locus were as follows. The length of the probe used and the restriction enzymes used to generate that probe are given in the parentheses: Region α: 9 kb BamHI Fragment (0.7 kb, HindIII/EcoRI fragment); region β: 5.9 kb BamHI/EcoRI fragment (0.7 kb EcoRI/EcoRV fragment); region γ: 5 kb EcoRI fragment (1.3 kb EcoRI/EcoR fragment); region δ: 4.2 kb EcoRI fragment (1.6 kb HindIII/EcoRI fragment); region ε: 11 kb BamHI fragment (1.2 kb EcoRI/BamHI fragment); region ζ: 13.5 kb EcoRI fragment (0.6 kb Xbal/ EcoRI fragment); region η: 3.7 kb XbaI fragment (0.9 kb Spel/Xbal fragment); region θ: 7.5 kb BamHI fragment (1.3 kb BamHI/EcoRI fragment).

Nuclei from the thymus, spleen and liver were digested with increasing amounts of DNase I. DNA was then purified, digested with appropriate restriction enzymes and analyzed by Southern blotting (FIG. 9B). Three groups of DNaseI HSS were identified (FIG. 9A). The first group contains clusters α, β, γ and δ which lie upstream of the first translated exon, two of which (β and γ) flank the untranslated exons and contain putative promoters. The second group lies in the largest intron between exons 2 and 3 and is comprised of clusters ε, ζ, and η. The third group is comprised of only one weak HSS θ in the immediate vicinity of the Ikaros polyadenylation site in the last exon. The DNaseI HSS within each cluster are indicated by vertical arrows shown in FIG. 9A which also designate their specificity for the thymus, spleen or for both.

In summary, the chromatin structure of the Ikaros gene appears to be disrupted in a tissue-specific manner in thymocytes and splenocytes in eight distinct clusters of DNaseI HSS. Four of these DNaseI HSS clusters are located upstream of exon 1 and two of these lie in the vicinity of the Ikaros promoters. Another three clusters lie in the intron between exons 2 and 3. These tissue specific regions of accessible chromatin are potentially the sites of action of hemo-lymphoid nuclear proteins and remodeling complexes that potentate the complex pattern of Ikaros gene expression in a variety of cell types of the hemo-lymphoid system.

B cell and Neutrophil-specific Activities of the Ikaros Promoter Regions

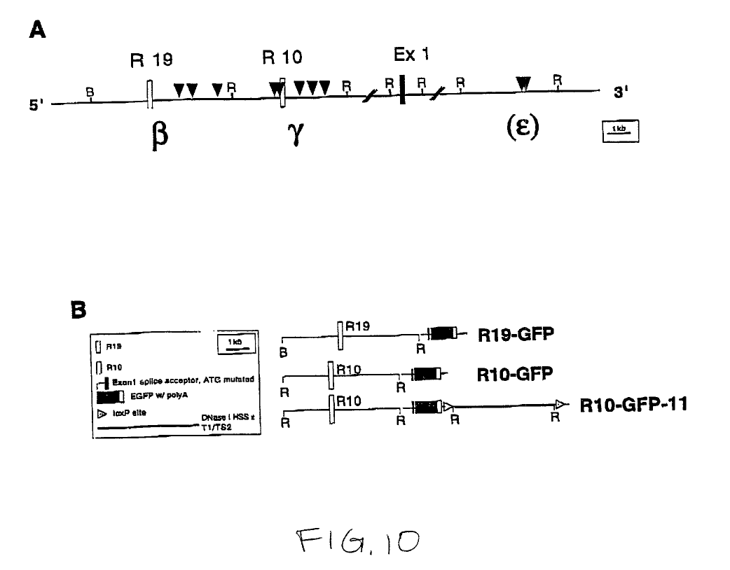

Regions that encompass sequences upstream and downstream of exons R10 and R19 and the associated β and γ DNaseI HSS clusters were tested for activity in transgenic mice (FIG. 10). The constructs including the β or γ clusters were made as follows. A genomic fragment encompassing 480 bp upstream exon I up to one base pair upstream of the start of translation was PCR amplified with primer 5′ Ex1BHI and 3′ ExlAgeI. These primers had linkers at their 5′ end to enable the cloning of the product into-pEGFP-1 (Clontech) after digestion with BamHI and AgeI. The resulting construct had 480 bp of exon 1 splice acceptor sequence upstream of the E-GFP-1 gene and is referred to as pEGFP-splice. At the 5′ end of the construct was an endogenous EcoRI site and at the 3′ of the SV40 poly adenylation signal was an AflII site.

5′ Ex1BH1 primer sequence (non hybridizing sequence underlined): aaa gga tcc gaa cat aac tat gga tca gcc (SEQ ID NO:______).

3′ ExAgeI primer sequence (no hybridizing sequence underlined): ttt acc ggt gtc ttc agg tta tct cct gc (SEQ ID NO:______).

DNase I HS region β was subcloned into Bluescript II SK (Stratagene) as a 5.9 kb BamHI/EcoRI fragment. pEGFP-splice was cloned at the 3′ end utilizing the EcoRI and ClaI (Bluescript)/AflIII (pEGFP-splice) sites. The cohesive ends of ClaI and AflIII were blunted using the Klenow fragment of E. coli DNA Polymerase I. This resulted in the R19-GFP construct. The insert was released from the vector backbone in a BamHI/XhoI double digest and prepared for microinjection.

DNase I HS region γ was subcloned into Bluescript II KS (Stratagene) as a 5 kb EcoRI fragment. pEGFP-splice was cloned at the 3′ utilizing the engineered BamHI and Spel (Bluescript)/AflII (pEGFP-splice) sites. The cohesive ends of Spel and AflIII were blunted using the Klenow fragment of E. coli DNA Polymerase I. This resulted in the R10-GFP construct. The insert was released from the vector backbone in a XhoI/SacII double digest and prepared for microinjection.

The activity and tissue specificity of these promoter regions was examined by following their ability to drive expression of a GFP reporter in a variety of blood cells. The exon 1 splice acceptor site was included downstream of the R10 and R19 exons as shown in FIG. 10B. The ATG start codon of Ikaros present in Exon 1 was mutated, and the E-GFP-1 cDNA was cloned at its 3′. Two series of transgenic founders were generated using these promoter-reporter constructs which arc referred to as R19-GFP and R10-GFP (FIG. 10B and Table 3).

Transgenic mice were made through an oocyte injection protocol as described (find reference). The mice were bred and maintained under sterile conditions in a pathogen-free animal facility at Massachusetts General Hospital. Mice were 4-8 weeks of age at the time of analysis. The mice were genotyped for GFP by PCR analysis using the following primer combination: GFPup3: cgt aaa egg cca caa gtt ca GFPdown3: ctt gaqa gtt cac ctt gat gc. Cycling conditions were: 95° C. 5 min, 80° C. add Taq, (94° C. 45 sec., 58° C. 45 set, 72° C. 45 sec.)×28,720C. 10 min., 4° C. until taken out.

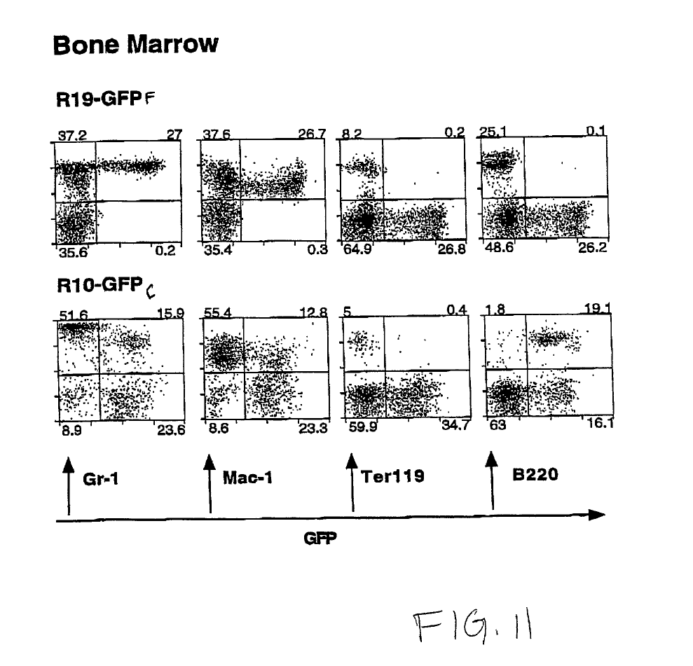

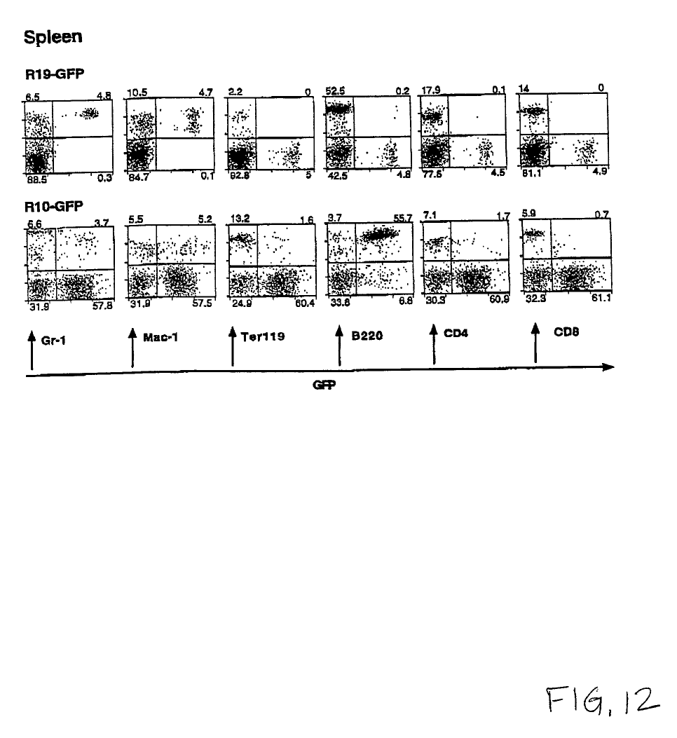

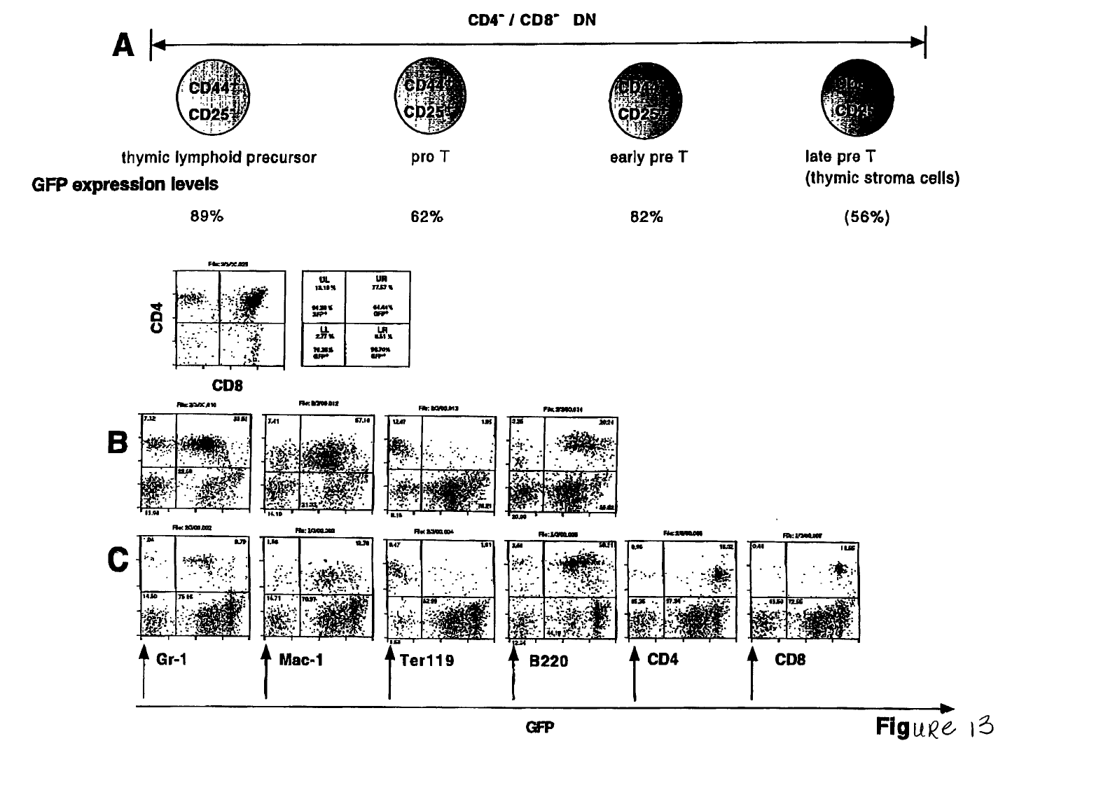

Four out of the eight R19-GFP founder lines express the reporter in a small subpopulation of the spleen and the bone marrow (Table 3,0.8-4.8%of splenocytes and 0.8-27% of bone marrow cells) that displays a high FSC/SSC. Staining with lineage specific markers revealed that in both tissues these cells are neutrophils (Table 3 and FIGS. 11 and 12, R19-GFP, Mac-1⁺, Gr-1⁺. Indeed among myeloid cells, Ikaros is normally expressed in terminally differentiated neutrophils. Morgan et al. (1997) EMBO J. 16(8):2004-2013; Klug et al. (1998) Proc. Natl Acad. Sci. USA 95(2):657-662. In the four R19-GFP founder lines, the expressing neutrophil population ranges from 1.7-41.58 (Table 3). This shows that the R19 promoter activity is specific for neutrophils and is subject to variegation effects, which are dependent on the site of its integration (FIG. 11, R19-GFP). Nonetheless, among different founder lines, the variegating neutrophil population expresses similar levels of GFP. In the analysis of the R19-GPP F37 line shown in FIGS. 11 and 12, approximately 41.5% of the neutrophils in the bone marrow and spleen express the reporter. The remaining four out of the eight R19-GFP founder lines did not express the reporter in any hemo-lymphoid or other cell type.

Cells from the thymus, spleen, and bone marrow were prepared and analyzed for expression of surface differentiation antigens as described previously (Georgopoulos (1994) Cell 79(1):143-56; Winandy et al. (1995) Cell 83(2):289-99). Flow cytometric analysis was performed using a Becton Dickinson FACScan flow cytometer and CellQuest software (Becton Dickinson, San Jose, Calif.) or the high speed MoFlo sorter (Cytomation, Inc.). All antibodies used for flow cytometric analysis were directly conjugated with fluorochromes of choice (all from PharMingen, San Diego, Calif.). GFP expression was directly detected under FITC laser settings.

Expression was also seen in eight out of eleven R10-GFP founders, but here the majority of GFP+ cells fall within the lymphoid gate. Analysis with lineage specific markers revealed that these cells were B cells in both the bone marrow and spleen (Table 3, 10-GFP). Among the different founders, the range of expressing cell population (GFP+) varied from 0.7-62% in the spleen and from <1-36.5% in the bone marrow. In all of the R10-GFP founder lines analyzed the great majority of GFP+ cells (89-98%) were cells of the B lineage (B220+) in the spleen (89-98%) and in the bone marrow (54%). A smaller fraction of GFP+ cells were neutrophils (4.6-35.5%) between spleen and bone marrow) (Table 3, 10-GFP). For the R10GFP line shown in FIGS. 11 and 12, 91-94% of bone marrow and splenic B cells (B220+) and 19-48% of neutrophils (Mac-1⁺/Gr-1⁺) were GFP+. Conversely, 89% of GFP+ splenocytes and 54% of GFP+ bone marrow cells were B cells and 8-35.5% neutrophils.

Thus, the R10 and R19 promoter regions appear to differ significantly in their cell type specificity. Whereas the activity of R19 is restricted to neutrophils, R10 is active in B cells and in a smaller fraction of neutrophils. Activity of both promoter regions in both populations is subject to position effect variegation indicating the lack of a locus control region (LCR).

An Intronic DNAseI HSS Cluster Diversifies Expression of the Ikaros B Cell and Neutrophil-specific Promoter to the T Cell Lineage

Although Ikaros is normally expressed in B cells and neutrophils, it is also expressed at its highest levels in differentiating thymocytes and mature T cells. Georgopoulos (1997) Curr. Opin. Immunology 9(2):222-227. Thus, additional regulatory elements must work in concert with the Ikaros promoter regions to direct expression in the T lineage. To determine the regulatory region(s) responsible for the Ikaros-T cell specific activity, the transcriptional potential of one of the most prominent DNaseI HSS present in the Ikaros locus in both the thymus and spleen was tested. A 4.7 kb EcoRI fragment containing two out of the three (T1/TS2) DNaseI HSS sites present in the ε cluster was introduced at the 3′ end of the R10-GFP reporter (FIG. 10B, R10-GFP-11). Briefly, the construct for transgenic line R10-GFP-11 was generated as follows. The R10-GFP construct was modified so that it no longer contained a KpnI site at the 5′ of the gene. Additionally, a KpnI site was introduced between the SacII and SacI sites at the 3′ of the construct. This resulted in construct R10-GFP-11. A loxP site containing vector was generated by cloning a loxP site between SalI and HindII and another loxP site between BamHI and XbaI of Bluescript II KS. For that, two annealed oligonucleotide were generated that contained a Sail cohesive end and a HindIIX cohesive end flanking a loxP site (see sequences 5′ top and 5′ bottom). Similarly, two other oligonucleotides were generated and annealed that contained a BamHI and an XbaI site flanking the loxP sequence (see sequences 3′ top and 3′ bottom). This resulted in vector BS-loxP. DNase I HS ε T1/TS2 was subcloned as a 4.6 kb EcoRI fragment into BS-loxP in 3′ to 5′ orientation. This resulted in construct BS-loxP-11. Subsequently, BS-IoxP-11 was digested with SacII and KpnI and cloned in an equally digested R10-GFP-mK. This resulted in construct R10-GFP-11. The insert was released from the vector backbone in a SalI digest and prepared for microinjection.

| | | |
|---|---|---|
| 5′top sequence: | tcg acg atc gat cga tcg atc ata act tcg tat aat gta tgc tat | (SEQ ID NO:_). |
| | acg aag tta tta agc tt | |
| 5′bottom sequence: | gat cca taa ctt cgt ata atg tat gct ata cga agt tat tt | (SEQ ID NO:_). |
| 3′top sequence: | gat cca taa ctt cgt ata atg tat gct ata cga agt tat tt | (SEQ ID NO:_). |
| 3′bottom sequence: | cta gaa ata act tcg tat agc ata cat tat acg aag tta tgg atc c | (SEQ ID NO:_). |

The transgenic mice were generated as described above.

Six out of the eight founder lines generated expressed GFP in the spleen, thymus and bone marrow (Table 3, 1 0-GFP- 11, expression range in the spleen from 1.7-91 %).

All mice used for this study were from the transgenic line R10-GFP-1 1, at 4-8 weeks of age. Thymic single cell suspensions were prepared as described previously [Winandy et al. (1999) J. Exp. Med. 190(8):1039-48. Thymocytes from 4-6 animals were pooled and depleted Mac-1, Terr119., B220, CD4 and CD8 ceils using magnetic beads coated with anti rat Fc goat (Paesel and Lorei, Duisburg, Germany). The depleted population was restained with PE-lineage Antibodies and sorted for PE negative cells using a MoFlo high speed cell sorter. The resulting cells were stained with CD43(Cychrome)and CD25 (PE) and analyzed as described earlier (Winandy et al. (1999) J. Exp. Med. 190(8):1039-48.

Analysis of thymocyte populations in the R10-GFP-11 F225 line is shown in FIG. 13. GFP expression is seen in 76% of the CD4⁻/CD8⁻, in 64% of the CD4⁺/CD8⁺ and in 94% and 97% of the CD4⁺ and CD8⁺ cells, respectively. In sharp contrast to the R10-GFP-11 lines, no significant expression among the thymocyte populations of the R10-GFP lines was seen (data not shown). Reporter activity within the immature thymocyte compartment was analyzed further. Expression of GFP was detected in the majority of the T cell progenitor/precursor populations (FIG. 13A, 89%of CD44⁺/CD25⁻, 62% of CD44⁺/CD25⁺, 82% of CD44⁻/CD25⁺).

In the spleen of the R10-GFP-11 F225 line shown in FIG. 13C, 92%of B cells and 89% of neutrophils were also positive. In addition, 97% of the CD4⁺/TCR⁺ and 99% of the CD8⁺/TCR⁺ T cells were positive for GFP. Significantly, expression in the T cell populations was approximately eight fold higher than in B cells and neutrophils (FIG. 13C, compare GFP+: B220 vs. CD4 or CD8), thereby recapitulating the higher levels of Ikaros expression in the T lineage. Georgopoulos (1997) Curr. Opin. Immunology 9(2):222-227.

Another difference in the activity of the R10-GFP and R10-GFP-11 reporter lines was noted within the neutrophil population. A greater percentage of neutrophils in the R10-GFP-11 (0.4-100%) vs. the R10-GFP (0.2-48%) lines expressed GFP. In the highest expressing R10-GFP vs. R10-GFP-11 founder lines, 48% vs. 100% of the Gr-1⁺/Mac-1⁺ populations was GFP+ (FIGS. 11-13).

In contrast to the T and neutrophil populations, GFP expression in the B lineage remained unchanged in the presence of the ε DNase I HSS cluster. Among the R10-GFP and R10-GFP-11 lines, the range of bone marrow and splenic B cells that were GFP+ was similar (Table 3, 1.4-94% vs. 1.5-94%). In both lines of transgenic founders, GFP expression in the B lineage was detected from the pro B (B220⁺/CD43⁺) cell stage on (data not shown).

In summary, transgenic mice that express the GFP reporter under the control of various transcriptional control elements associated with three DNAseI HSS clusters within the Ikaros locus have been generated. It was shown that B cell and neutrophil specificity for regions associated with two independently utilized promoters and an intronic enhancer region that diversifies one of the Ikaros promoters into T cells and gives it a higher level of activity was identified.

Differential Labeling of T Versus B Cell Zones by the Ikaros Regulatory Regions

The ability of Ikaros-GFP reporters to demarcate lymphoid populations, the sites of their emergence and action is examined by fluorescence microscopy. At a macroscopic level no apparent staining has been detected with the neutrophil specific R19-GFP lines, possibly due to the small number of GFP+ cells present in lymphatic centers (Table 3,0.8-4.8%). However, in both of the higher expressing R10-GFP and R10-GFP-11 lines, prominent staining of the lymphoid organs was seen. In the case of the R10-GFP lines, the B cell follicles of the spleen and peripheral lymphatics are prominently demarcated whereas the T cell zones remain negative.

In the R10GFP-11 lines, the T cell zones show the most prominent staining with B cell follicles also staining but at a lower level. This clearly reflects the expression pattern of these reporters in the T versus B cell populations. In addition to the spleen and lymph nodes, the thymus and bone marrow were also strongly positive in the R10-GFP-11 line.

Ikaros Auto Regulation of the R10 Promoter Region in B Cells

Sequence analysis of the Ikaros R10 promoter region revealed a number of Ikaros binding sites. The possibility of auto regulation for this region was examined by breeding the Ikaros R10-GFP reporter lines onto the Ikaros null and dominant negative mutations. In the absence of one Ikaros functional allele an increase in GFP levels per cell was detected with the R10-GFP founder line (F76) in which expression in 94%of the B cell population is detected. The increase in GFP levels was on average 3 fold in the pre-B and B cell population of the bone marrow and five fold in the mature B cell Population of the spleen. In contrast to the increase in GFP levels in B cells, no significant change was detected in the non-B cell GFP+ population of the bone marrow and spleen which in its majority consists of neutrophils. The same effect on R10-GFP levels was also seen upon breeding to the Ikaros DN+/− background. A second R10 founder line in which only 60% of B cells were GFP+ was also bred to the Ikaros mutations (Table 3, F30). Two effects were seen with this line of mice having the Ikaros DN+/− background: levels of GFP increased per cell and the expressing B cell population increased from 60%to 90%.

Thus Ikaros has two distinct effects on the B cell specific elements of the R10 promoter. On one hand the transcriptional activity of the R10 promoter region integrated in a permissive chromatin environment appears to be regulated in a negative fashion by Ikaros. When integrated at a site where chromatin is less permissive and is subject to variegation effects then Ikaros influences both variegation as well as levels of transcription. These effects are not seen with the transcriptional elements that confer neutrophil-specific activity to the Ikaros R10 promoter region.

TABLE 3
Expression of GFP Under Transcriptional Control of Various Ikaros
Regulatory Elements in the Spleen and Bone Marrow

| | Spleen | % | % | | % | | % | Bom | % | % | | % | |
|---|---|---|---|---|---|---|---|---|---|---|---|---|---|
| | +ve | Mac1 | GFP+ve | % B | GFP+ve | % T | GFP+ve | +ve | Mac1 | GFP+ve | % B | GFP+ve | % T |
| 10- | | | | | | | | | | | | | |
| GFP | | | | | | | | | | | | | |
| F28 | 0.7 | 0.2m | 4.6m | 1.4 | 98.3 | 0 | 0.0 | nd | nd | nd | nd | nd | nd |
| F30 | 35 | 19.4m | 7.8m | 68 | 93.5 | 4.2 | 4.0 | nd | nd | nd | nd | nd | nd |
| F76 | 62.2 | 48.6 | 8.3 | 93.8 | 89.1 | 15 | 1.9 | 36.5 | 18.8 | 35.5 | 91.4 | 54.3 | nd |
| 19- | | | | | | | | | | | | | |
| GFP | | | | | | | | | | | | | |
| F45 | 0.8 | 9.2 | 93.4 | 0 | 0 | 0 | 0.0 | 2.2 | 6.7 | 98.5 | 0 | 0 | nd |
| F63 | 0.3 | 2.86 | 95 | 0 | 0 | 0 | 0.0 | 0.8 | 1.7 | 98.8 | 0 | 0 | nd |
| F35 | 0.3 | 5.8 | 81.4 | 0 | 0 | 0 | 0.0 | 2 | 5.36 | 96.8 | 0 | 0 | nd |
| F37 | 4.8 | 30.9 | 97.9 | 0.4 | 4 | 0 | 0.0 | 26.9 | 41.5 | 98.9 | 0.4 | 0.4 | nd |
| 10- | | | | | | | | | | | | | |
| GFP- | | | | | | | | | | | | | |
| 11 | | | | | | | | | | | | | |
| F202 | 91 | 99.4 | 8.2 | 89.4 | 38.8 | 97.5 | 15.2 | nd | nd | nd | nd | nd | nd |
| F214 | 52 | 95.33 | 15.7 | 93.5 | Sk | >95 | Sk | nd | nd | nd | nd | nd | nd |
| F225 | 84 | 89.1 | 15.2 | 91.7 | 47.1 | 95.5 | 16.6 | 77.75 | 88.5 | 72.8 | 86.2 | 26.4 | nd |
| F226 | 1.7 | 0.4 | 3.3 | 1.5 | 53.1 | 1.7 | 39.7 | nd | nd | nd | nd | nd | nd |
| F215 | 60.26 | 50.3 | 15.2 | 63.5 | 63.1 | 75.6 | 10.5 | nd | nd | nd | nd | nd | nd |

Discussion

Ikaros has previously been shown to be essential for development and homeostasis in the hemo-lymphoid system. Mutations in the Ikaros gene that interfere with its normal levels of expression cause a range of hematological disorders including immunodeficiencies as well as leukemias and lymphomas. It was found that there is a number of key regulatory regions in the Ikaros genomic locus whose combinatorial action recapitulate the complex pattern of Ikaros expression during differentiation in the B-and T-lymphoid and myeloid lineages. Importantly, a subset of these elements that confer B cell specific expression are subject to auto regulation.

The Ikaros genomic locus spans approximately 120 kB and is comprised of two untranslated and seven translated exons. Eight putative regulatory regions were mapped within this locus using a DNaseI HSS approach. These tissue specific DNAseI HSS demarcate regions of chromatin that are uniquely accessible in differentiating thymocytes and/or in splenocytes. Accessibility in these chromatin regions most likely reflects the activity of developmentally regulated transcription factors which function by recruiting remodeling factors to potentate transcription of Ikaros in different cell types of the lymphoid and hematopoietic system. Significantly, one of these clusters (DNase I HSS ε) is frequently found in the vicinity retroviral integrations associated with leukemias. This may underlie changes in its activity and cause the disease state.

Two putative promoters were mapped in the Ikaros locus in the vicinity of two of the tissue specific DNaseI HSS clusters. One of the promoter regions was only active in neutrophils (R19), whereas the second (R10) was active predominantly in B cells as well as in neutrophils. Activity of the R10 promoter region was noted in the early pro-pre-B cells in the bone marrow and was maintained in mature B cells in the periphery. Within both B and neutrophil populations, a variegation in the activity of promoter regions was seen, indicating that these were subject to position effects caused by the local chromatin. Thus, additional elements with insulator function that protect from restrictive effects of neighboring chromatin must be present in the Ikaros locus to allow for its consistent expression in the majority of B cells and neutrophils. Festenstein et al. (1996) Science 271(5252):1123-5; Kioussis et al. (1997) Curr. Opin. Genet. Dev. 7(5):614-9.

Neither of the two Ikaros promoter regions were active in T cells that normally express high level of Ikaros, which is critical for their regulated proliferation and homeostasis. However, the B cell/neutrophil-specific promoter region combined with the intronic ε DNaseI HSS cluster was highly active in T cells. Under the control of the ε enhancer region, expression was restored in the earliest double negative thymic precursors as well as in the double positive and single positive thymocytes and in peripheral T cells. Significantly, expression in cells of the T lineage was by approximately one order of magnitude greater than in B cells and neutrophils recapitulating expression of the endogenous Ikaros. Georgopoulos (1997) Curr. Opin. Immunology 9(2):222-227. Furthermore, this combination of promoter and intronic DNaseI HS S cluster regions increased the number of expressing neutrophils, relative to that detected with either of the Ikaros promoter regions (R10 or R19) alone. However, variegation of expression among the lymphoid and myeloid populations was still detected with this combination of promoter and enhancer elements, indicating that critical insulator elements were still missing. Insulators may be present in one or more of the four clusters of DNase I HSS that are under investigation. Nonetheless, the B cell/neutrophil specific promoter region when acting in concert with the ε intronic enhancer(s) is active in a pattern that closely resembles that of the endogenous Ikaros expression in the hemo-lymphoid system.

Many key transcriptional regulators are under positive and negative feed back control mechanisms that ensure their production at appropriate levels in support of normal differentiation. Regulation of Ikaros levels is of paramount importance for the proper development of the hematopoietic and immune systems and it appears to follow a negative feed back loop at least in cells of the B lineage. Ikaros negatively regulates the activity of its own B cell specific promoter elements integrated at sites of permissive chromatin. A greater expression (6-3 fold) is detected within pre-B and B cell populations when Ikaros levels are reduced. When these elements are integrated at sites where position effects are manifested, variegation is decreased upon Ikaros reduction. Both of these Ikaros effects on its own B cell specific regulatory elements can be explained by changes in the chromatin status. Recruitment of Ikaros at cognate binding sites present in this regulatory region may restrict the chromatin environment and reduce its overall transcriptional activity. A more severe reduction may be manifested at specific chromosome locations which are already in a more restricted conformation. This can lead to shut down in expression in a significant fraction of B cells. This Ikaros negative auto-regulation seems to be specific for the B cell restricted regulatory elements and is not detected with the neutrophil-restricted elements present in the same promoter region. These studies provide an insight into the function of Ikaros as a negative regulator of transcription in vivo and its ability to target its own locus.

Markers which can distinguish between stem cells, various multipotent and oligopotent progenitors, and lineage-restricted precursors are of paramount importance for stem cell biology. Given its early hematopoietic pattern of expression, Ikaros is an excellent candidate for dissecting the early hematopoietic hierarchy, in addition to probing its molecular regulation. The Ikaros expression cassettes described herein are comprised of subsets of its regulatory elements, which may allow for labeling and therefore distinguish between subsets of hemo-lymphoid cells. GFP reporters driven by these regulatory elements may also provide a way to address the ontogeny, migration properties of progenitors and precursors and the sites of action of their mature progeny in real time in the intact organism. They will also provide powerful tools to direct expression at stages of the hematopoietic system like the HSC and the early myeloid and lymphoid progenitors and precursors, that have been difficult to target so far and provide molecular intervention in these rare cell types.

Delineation of the Ikaros regulatory elements in normal and Ikaros deficient mouse models will provide a molecular understanding of the mechanisms that underlie the development of immune and hematological diseases in mice and men.

Other Embodiments

Nucleic acid encoding all or part of the Ikaros gene can be used to transform cells. For example, the Ikaros gene, e.g., a mis-expressing or mutant form of the Ikaros gene, e.g., a deletion, or DNA encoding an Ikaros protein can be used to transform a cell and to produce a cell in which the cell's genomic Ikaros gene has been replaced by the transformed gene, producing, e.g., a cell deleted for the Ikaros gene. As described above, this approach can be used with cells capable of being grown in culture, e.g., cultured stem cells, to investigate the function of the Ikaros gene.

Analogously, nucleic acid encoding all or part of the Ikaros gene, e.g., a mis-expressing or mutant form of the gene, e.g., a deletion, can be used to transform a cell which subsequently gives rise to a transgenic animal. This approach can be used to create, e.g., a transgenic animal in which the Ikaros gene is, e.g., inactivated, e.g., by a deletion. Homozygous transgenic animals can be made by crosses between the offspring of a founder transgenic animal. Cell or tissue cultures can be derived from a transgenic animal. A subject at risk for a disorder characterized by an abnormality in T cell development or function, e.g., leukemia, can be detected by comparing the structure of the subject's Ikaros gene with the structure of a wild type Ikaros gene. Departure from the wild type structure by, e.g., frameshifts, critical point mutations, deletions, insertions, or translocations, is indicative of risk. The DNA sequence of the coding region of several exons as well as several intron exon boundaries are included herein. Other regions can be obtained or sequenced by methods known to those skilled in the art.

Embodiments of the invention also include animals having an Ikaros transgene and a second transgene which allows control over the expression of the Ikaros gene.

In vivo site-specific genetic manipulation together with genetic crosses between transgenic animals can be used to make animals which express the subject Ikaros protein in a developmentally regulated or tissue-specific manner. It is often desirable to limit the expression of a transgene to a particular stage of development or to a specific tissue. For example, many transgenes have deleterious effects on the cells of the transgenic animal in which they are expressed; thus, it is difficult to construct transgenic animals expressing these genes. Also, many promoters are “leaky” resulting in minimal levels of transcription of their target gene in all cell types. In many instances, it is desirable for a gene to be tightly repressed in all cells except those of a specific tissue. It may also be useful to study the role of a particular gene in development by causing or preventing its expression in particular tissues or at particular stages of development. One approach to the regulation of transgenes involves control of gene expression in vivo in either a tissue-specific manner or at a specific stage of the animal's development via site-specific genetic recombination.

Genetic techniques which allow for the expression of transgenes can be regulated via site-specific genetic manipulation in vivo are known to those skilled in the art. Genetic systems are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of the subject protein. For example, excision of a target sequence which interferes with the expression of the subject protein can be designed to activate expression of that protein. This interference with expression of the subject protein can result from a variety of mechanisms, such as a spatial separation of the subject protein gene from the promoter element resulting in the inhibition of transcription of the Ikaros gene. In another instance, a target sequence containing an internal stop codon can be used to prevent translation of the subject protein. Alternatively, in situations where the target sequence comprises the subject gene coding sequence or the promoter element, recombinase catalyzed excision can be used to inhibit expression of the subject protein via excision of these sequences. Nucleic acid constructs can also be made wherein a target sequence containing a sequence encoding the subject protein is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The cre/loxP recombinase system of bacteriophage P1 (Lakso et al. PNAS 89:6232-6236; Orban et al. PNAS 89:6861-6865) and the FLP recombinase system of Saccharomyces cerevisiae (O'Gorman et al. Science 251:1351-1355; PCT publication WO 92/15694) are examples of in vivo site-specific genetic recombination systems known in the art. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. J. Biol. Chem. 259:1509-1514). The Cre recombinase catalyzes the excision of the target sequence when the loxP sequences are oriented as direct repeats and catalyzes inversion of the target sequence when loxP sequences are oriented as inverted repeats.

Use of the cre/loxP recombinase system to regulate expression of the Ikaros protein requires the construction of a transgenic animal containing transgenes encoding both the Cre recombinase and the subject protein. Mice containing both the Cre recombinase and the subject protein genes can be provided through the construction of double transgenic mice. A convenient method for providing such mice is to mate two transgenic animals each containing a transgene. Double transgenic progeny of this mating are identified by screening the resulting offspring for the presence of both transgenes. The progeny may be tested for the presence of the constructs by Southern blot analysis of a segment of tissue. Typically, a small part of the tail is used for this purpose.

Recombinant vectors can be constructed wherein the nucleic acid sequence encoding the Ikaros protein is separated from a promoter element, e.g., a constitutive promoter, by an target sequence flanked by loxP sequences. This excisable target sequence can be used to inhibit expression of the Ikaros protein by, for example, containing an internal stop codon. In such a case, expression of the subject protein will be activated in cells containing Cre recombinase activity by excision of the target sequence and ligation of the abutting sequences. In this instance, excision of the target sequence results in the activation of protein expression at the level of translational. Alternatively, the target sequence can be placed in such a position that Cre recombinase mediated excision results in the promoter element being brought into close enough proximity to the subject gene to confer transcriptional activation. In this instance, the target sequence inhibits transcription of the subject protein gene by spatially separating the promoter element from the coding sequence. In another construct, the target sequence can comprise the nucleic acid sequence encoding the Ikaros protein which is oriented in a 3′ to 5′ with respect to the promoter. In this orientation the promoter will not be capable of activating transcription of the subject nucleic acid sequence. In this instance, Cre recombinase will catalyze the inversion of the target sequence encoding the Ikaros protein and thereby bring the 5′ region of the coding sequence into the proper orientation with respect to the promoter for transcriptional activation.

In each of the above instances, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation or inactivation expression of the Ikaros protein can be regulated via regulation of recombinase expression.

Suitable recombinant vectors can be produced, for example, wherein a gene encoding the Cre recombinase is operably linked to a tissue-specific promoter, e.g., the mouse lck promoter which activates transcription in thymocytes. Tissue-specific expression of the Cre recombinase in each of the instances given above will result in a corresponding tissue-specific excision of the target sequence and activation or inactivation of the expression of the subject protein in that particular tissue. Thus, expression of the Ikaros protein will be up- or down-regulated only in cells expressing Cre recombinase activity.

One advantage derived from initially constructing transgenic mice containing a nucleotide sequence encoding the subject protein in a Cre recombinase mediated expressible format is evident when expression of the subject protein is deleterious to the transgenic animal. In such an instance, a founder population, in which the subject transgene is silent in all tissues, can be maintained. Individuals of this founder population can be crossed with animals expressing the Cre recombinase in, for example, one or more tissues. Thus, the creation of a founder population in which the subject transgene is silent will allow the study of genes which when expressed confer, for example, a lethal phenotype.

In instances where expression of the subject protein is not highly deleterious to the transgenic animal, tissue-specific gene activation systems similar to those described above can be devised which employs transgenic mice transfected with a single nucleic acid molecule. In such instances, the Cre recombinase and the nucleotide sequence encoding the subject protein are carried by the same vector and are integrated at the same chromosomal locus. Since the Cre recombinase is a trans-acting factor, the recombinase and the gene for which tissue-specific transcriptional activation is desired may be integrated at the same or different locations in the host genome.

Moreover, a tissue-specific promoter can be operably linked to more than one nucleic acid sequence, each encoding a different protein. In addition, more than one nucleic acid sequence containing a target sequence which inhibits protein expression, for example, can be introduced into cells. Thus, if desired, the subject Ikaros protein can be co-expressed with other transgenes where the expression of each protein is regulated in a tissue-specific or developmental stage-specific manner.

All of the above-cited references and publications are hereby incorporated by reference.

Other embodiments are within the following claims.

What is claimed is:

1. An isolated Ikaros transcriptional control region comprising one or more Ikaros regulatory element.

2. The Ikaros transcriptional control region of claim 1, comprising all or a functional fragment of a promoter of the β cluster.

3. The Ikaros regulatory control region of claim 1, comprising all or a functional fragment of a promoter of the γ cluster.

4. The Ikaros regulatory control region of claim 2, further comprising all or a functional fragment of a promoter of the γ cluster.

5. The Ikaros regulatory control region of any of claims 2, 3 or 4, further comprising one or more Ikaros regulatory element from the a cluster, the ε cluster, the η cluster or the θ cluster.

6. The Ikaros regulatory control region of claim 4, further comprising the ε cluster or a portion thereof.

7. A DNA construct comprising an Ikaros transcriptional control region of claim 1 and a sequence encoding a reporter molecule.

8. The DNA construct of claim 7, wherein the reporter molecule is a reporter molecule which can luminesce or fluoresce.

9. The DNA construct of claim 7, wherein the reporter molecule is selected from a beta-galactosidase gene, a luciferase gene, a green fluorescent protein gene, an alkaline phosphatase gene, a horseradish peroxidase gene, and a chloramphenicol acetyl transferase gene.

10. The DNA construct of claim 7, wherein the reporter molecule is green fluorescent protein.

11. A transgenic animal, or cell or tissue therefrom, comprising a transgene includes an Ikaros transcriptional control region operably linked to a sequence which is functionally unrelated to the Ikaros gene.

12. The transgenic animal of claim 11, wherein the animal is a rodent.

13. The transgenic animal of claim 12, wherein the rodent is a mouse.

14. The transgenic animal of claim 11, wherein the Ikaros transcriptional control region includes one or more Ikaros regulatory element.

15. The transgenic animal of claim 11, wherein the Ikaros transcriptional control region comprises the β cluster or a functional fragment of the promoter of the β cluster.

16. The transgenic animal of claim 11, wherein the Ikaros transcriptional control region comprises the γ cluster or a functional fragment of the promoter of the γ cluster.

17. The transgenic animal of claim 15, wherein the Ikaros transcriptional control region further comprises the γ cluster or a functional fragment of the promoter of the γ cluster.

18. The transgenic animal of any of claims 14, 15, or 16, wherein the Ikaros transcriptional control region further comprises one or more Ikaros regulatory element from the α cluster or a portion thereof, the ε cluster or a portion thereof, the η cluster or a portion thereof, or the θ cluster or a portion thereof.

19. The transgenic animal of claim 15, wherein the Ikaros transcriptional control region further comprises the ε cluster or a portion thereof.

20. The transgenic animal of claim 19, wherein the Ikaros transcriptional control region comprises a portion of the ε cluster.

21. The transgenic animal of claim 11, wherein the sequence functionally unrelated to the Ikaros gene encodes a reporter molecule.

22. The transgenic animal of claim 21, wherein the reporter molecule is a reporter molecule which can luminesce or fluoresce.

23. The transgenic animal of claim 21, wherein the sequence encoding the reporter molecule is selected from a beta-galactosidase gene, a luciferase gene, a green fluorescent protein gene, an alkaline phosphatase gene, a horseradish peroxidase gene, and a chloramphenicol acetyl transferase gene.

24. The transgenic animal of claim 21, wherein the reporter molecule is green fluorescent protein or a variant thereof.

25. The transgenic animal of claim 24, wherein the reporter molecule is a variant of green fluorescent protein.

26. The transgenic animal of claim 25, wherein the variant of green fluorescent protein is selected from the group consisting of EGFP, EBFP, EYFP, d2EGFP, ECFP, and GFPuv.

27. The transgenic animal of claim 1, wherein the genome of the animal further comprises an alteration by disrupting at least one exon of the endogenous Ikaros gene.

28. The transgenic animal of claim 27, wherein the endogenous Ikaros gene is disrupted by insertion of a nucleic acid sequence.

29. The transgenic animal of claim 28, wherein the insertion results in any of an inversion, deletion, translocation, or reciprocal translocation.

30. The transgenic animal of claim 28, wherein the insertion is in or alters the sequence, expression, or splicing of one or more of the following exons: exon 1/2, exon 3, exon 4, exon 5, exon 6, and exon 7.

31. The transgenic animal of claim 28, wherein the insertion is in or alters the sequence, expression, or splicing of a DNA binding domain of the Ikaros gene.

32. The transgenic animal of claim 28, wherein the insertion results in a deletion of portions of exon 3 and exon 4.

33. The transgenic animal of claim 28, wherein the animal is heterozygous for the insertion.

34. The transgenic animal of claim 28, wherein the animal is homozygous for the insertion.

35. The transgenic animal of claim 28, wherein the insertion is in a domain involved in transcriptional activation or in dimerization.

36. The transgenic animal of claim 28, wherein the insertion is in exon 7.

37. The transgenic animal of claim 11, wherein the genome of the animal further comprises an alteration by disrupting at least one exon of the endogenous gene encoding a protein involved in hematopoiesis.

38. The transgenic animal of claim 37, wherein the endogenous gene is disrupted by insertion of a nucleic acid sequence.

39. The transgenic animal of claim 38, wherein the endogenous gene encodes Helios.

40. The transgenic animal of claim 38, wherein the endogenous gene encodes Aiolos.

41. The transgenic animal of claim 38, wherein the insertion results in any of an inversion, deletion, translocation, or reciprocal translocation.

42. A method of evaluating the development of a component or a cell lineage of the immune system, comprising:
providing a transgenic animal of claim 11 or claim 37, or a cell or tissue therefrom; and
monitoring expression of the protein unrelated to Ikaros.

43. The method of claim 42, wherein the sequence functionally unrelated to the Ikaros gene encodes a reporter molecule.

44. The method of claim 43, wherein the reporter molecule is a reporter molecule which can luminesce or fluoresce.

45. The method of claim 43, wherein the sequence encoding the reporter molecule is selected from a beta-galactosidase gene, a luciferase gene, a green fluorescent protein gene, an alkaline phosphatase gene, a horseradish peroxidase gene, and a chloramphenicol acetyl transferase gene.

46. The method of claim 43, wherein the reporter molecule is green fluorescent protein or a variant thereof.

47. The method of claim 46, wherein the reporter molecule is a variant of green fluorescent protein.

48. The method of claim 47, wherein the variant of green fluorescent protein is selected from the group consisting of EGFP, EBFP, EYFP, d2EGFP, ECFP, and GFPuv.

49. The method of claim 43, wherein hematopoietic development is evaluated in a living animal.

50. The method of claim 49, wherein hematopoietic development is evaluated by detecting a fluorescent signal on the live animal.
